(19) United States
(12) Patent Application Publication
(10) Pub. No.: US 2015/0299178 A1
(43) Pub. Date: Oct. 22, 2015

(54) Thiazole Derivatives as Alpha 7 NACHR Modulators

(71) Applicant: LUPIN LIMITED, Mumbai, Maharashtra (IN)

(72) Inventors: Neelima Sinha, Pune (IN); Navnath Popat Karche, Pune (IN); Anil Kashiram Hajare, Pune (IN); Baban Rupaji Thube, Pune (IN); Firoj Aftab Raje, Pune (IN); Dnyaneshwar Changdeo Bhanage, Pune (IN); Sudhakar Maruti Balgude, Pune (IN); Venkata P. Palle, Pune (IN); Rajender Kumar Kamboj, Pune (IN)

(73) Assignee: Lupin Limited, Mumbai, Maharashtra (IN)

(21) Appl. No.: 14/439,120

(22) PCT Filed: Nov. 11, 2013
(86) PCT No.: PCT/IB2013/060056

§ 371 Date: Apr. 28, 2015

(30) Foreign Application Priority Data
Nov. 12, 2012 (IN) 1307/KOL/2012
Jan. 4, 2013 (IN) 10/KOL/2013
Jan. 4, 2013 (IN) 11/KOL/2013
Jan. 18, 2013 (IN) 62/KOL/2013
Jan. 24, 2013 (IN) 226/MUM/2013

(51) Int. Cl. C07D 417/06; A61K 45/06; A61K 31/4439; A61K 31/439; C07D 277/24; A61K 31/426

(52) U.S. Cl. CPC C07D 417/06; C07D 277/24; A61K 31/426; A61K 31/4439; A61K 31/439; A61K 45/06

(57) ABSTRACT

Disclosed is a compound of formula (I), wherein R¹, R², R³, R⁴, R⁵ and m are as described herein, as a modulator of nicotinic acetylcholine receptors particularly α7 subtype, its tautomeric forms, its stereoisomers, its pharmaceutically acceptable salts, its pharmaceutical composition, and its combinations with suitable medicaments. Also disclosed are a process of preparation of the compounds and the intended uses thereof in therapy, particularly in the prophylaxis and therapy of disorders such as Alzheimer's disease, mild cognitive impairment, senile dementia, and the like.

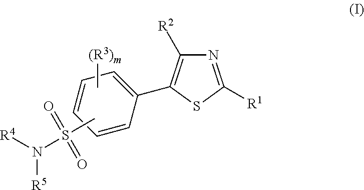

### FIELD OF THE INVENTION

The present invention relates to thiazole derivatives, their tautomeric forms, their stereoisomers, and their pharmaceutically acceptable salts, pharmaceutical compositions comprising one or more such compounds, and their use as nicotinic acetylcholine receptor α7 subunit (α7 nAChR) modulator.

### CROSS-REFERENCE TO A RELATED APPLICATION

The present application claims the benefit of Indian Provisional Patent Application Nos. 1307/KOL/2012 filed on 12^{th} Nov. 2012, 10/KOL/2013 filed on 4^{th} Jan. 2013, 11/KOL/2013 filed on 4^{th} Jan. 2013, 62/KOL/2013 filed on 18^{th} Jan. 2013, and 226/MUM/2013 filed on 24^{th} Jan. 2013 the disclosure of which is incorporated herein by reference in its entirety.

### BACKGROUND OF THE INVENTION

Cholinergic neurotransmission, mediated primarily through the neurotransmitter acetylcholine (ACh), is a predominant regulator of the physiological functions of the body via the central and autonomic nervous system. ACh acts on the synapses of the neurons present in of all the autonomic ganglia, neuromuscular junctions and the central nervous system. Two distinct classes of ACh target receptors viz. muscarinic (mAChRs) and the nicotinic (nAChRs) have been identified in brain, forming a significant component of receptors carrying its mnemonic and other vital physiological functions.

Neural nicotinic ACh receptors (NNRs) belong to the class of ligand-gated ion channels (LGIC) comprising of five subunits (α2-α10, β2-β4) arranged in heteropentameric (α4β2) or homopertameric (α7) configuration (David Paterson et al., Progress in Neurobiology, 61 (2000), 75-111). α4β2 and α7 nAChR constitute the predominant subtypes expressed in the mammalian brain. α7 nAChR has attained prominence as a therapeutic target due to its abundant expression in the learning and memory centers of brain, hippocampus and the cerebral cortex (F. Rubboli et al., Neurochem. Int., 1994, 25 (1), 69-71). Particularly, α7 nAChR is characterized by a high Ca²⁺ ion permeability, which is responsible for neurotransmitter release and consequent modulation of excitatory and inhibitory neurotransmission (Manickavasagom Alkondon et al., European Journal of Pharmacology, 393 (2000), 59-67; Federico Dajas-Bailador et al., TRENDS in Pharmacological Sciences, 2004, 25 (6), 317-324). Furthermore, high Ca²⁺ ion influx also has implications on the long-term potentiation of memory via alterations in gene expression (Robert S. Bitner et al., The Journal of Neuroscience, 2007, 27 (39), 10578-10587; Bruce E. McKay et al., Biochemical Pharmacology, 74 (2007), 1120-1133).

Several recent studies have confirmed the role of α7 nAChR in neural processes like attention, memory and cognition (Huibert D, Mansvel der et al., Psychopharmacology, (2006), 184, 292-305; Wai Kit Chan et al., Neuropharmacology, 52 (2007), 1641-1649; Jared W. Young et al., European Neuropsychopharmacology, (2007), 17, 145-155). Gene polymorphisms associated with the α7 nAChR protein CHRNA7 have been implicated in the genetic transmission of schizophrenia, related neurophysiological sensory gating deficits and resultant cognitive impairment (Robert Freedman et al., Biol. Psychiatry, 1995, 38, 22-33; Debby W. Tsuang et al., American Journal of Medical Genetics (Neuropsychiatric Genetics, 105, 662-668 (2001)). Also, preclinical studies in α 7 nAChR knock-out and anti-sense oligonucleotide treated mice have demonstrated impaired attention and defective cognition underscoring the prominent role of α7 nAChR in cognition (Peter Curzon et al., Neuroscience Letters, 410 (2006), 15-19; Jared W. Young et al., Neuropsychopharmacology, (2004), 29, 891-900). Additionally, pharmacological blockade of α 7 nAChR impairs memory and its activation enhances same in preclinical rodent models implicating α7 nAChR as target for cognitive enhancement (Kenji Hashimoto et al., Biol. Psychiatry, 2008, 63, 92-97).

Pathological brain function in sensory-deficit disorders has been associated with nicotinic cholinergic transmission particularly through α7 receptors (Robert Freedman et al., Biol. Psychiatry, 1995, 38, 22-33; T Debby W. Tsuang et al., American Journal of Medical Genetics (Neuropsychiatric Genetics, 105, 662-668 (2001); Robyn Carson et al., Neuromol., Med. (2008), 10, 377-384; S. Leonard et al., Pharmacology Biochemistry and Behavior, 70 (2001), 561-570; Robert Freedman et al., Current Psychiatry Report, 2003, 5, 155-161; Tyrone D. Cannon et al., Current Opinion Psychiatry, 2005, 18, 135-140). A defective pre-attention processing of sensory information is understood to be the basis of cognitive fragmentation in schizophrenia and related neuropsychiatric disorders (Steven C. Leiser et al., Pharmacology & Therapeutics, 122 (3), (2009), 302-311). Genetic linkage studies have traced sharing of the α7 gene locus for several affective, attention, anxiety and psychotic disorders (S. Leonard et al., Pharmacology, Biochemistry and Behavior, 70 (2001), 561-570; Suemaru K. Folia et al., Folia Pharmacol. Jpn., 119, 295-300 (2002)).

Perturbations in the cholinergic and glutamatergic homeostasis, has long been implicated as causative factors for host of neurological disease, including dementia(s) (Eran Nizri et al., Drug News Perspect., 2007, 20 (7), 421-429). Dementia is a severe, progressive, multi-factorial cognitive disorder affecting memory, attention, language and problem solving. Nicotinic ACh receptor, particularly the interaction of α7 receptor to αβ₁₋₄₂ is implicated as an up-stream pathogenic event in Alzheimer's disease, a major causative factor for dementia (Hoau-Yan Wang et al., The Journal of Neuroscience, 2009, 29 (35), 10961-10973). Moreover, gene polymorphisms in CHRNA7 have been implicated in dementia with lewy bodies (DLB) and Pick's disease (Agnes Feher et al., Dement. Geriatr. Cogn. Disord., 2009, 28, 56-62).

Disease modification potential of nAChRs particularly the α7 receptor has application for disease-modification of Alzheimer's disease (AD) and Parkinson's disease (PD) by enhancing neuron survival and preventing neurodegeneration (Hoau-Yan Wang et al., The Journal of Neuroscience, 2009, 29 (35), 10961-10973; R. G. Nagele et al., Neuroscience, 2002, 110 (2), 199-211; G. Jeyarasasingam et al., Neuroscience, 2002, 109, 275-285). Additionally, α7 nAChR induced activation of anti-apoptotic (BCL-2) and anti-inflammatory pathways in brain could have neuroprotective effects in neurodegenerative diseases (Mario B. Marrero et al., Brain Research, 2009, 1256, 1-7). Dopamine containing neurons of ventral tegmental area (VTA) and laterodorsal tegmental nucleus (LDT) are known to express nicotinic ACh receptors, particularly α4, α3, β2, β3, β4 subunits (Alexander Kuzmin et al., Psychopharmacology, (2009), 203, 99-108). Nicotinic ACh receptors, α4β2 and α3β4 have been identified with candidate-gene approach to have strong mechanistic link for nicotine addiction (Robert B. Weiss et al., PLoS Genetics, 2008, 4 (7), e1000125). α7 nAChR has particularly been studied for a putative role in cannabis addiction (Marcello Solinas et al., The Journal of Neuroscience, 2007, 27 (21), 5615-5620). Varenicline, a partial agonist at α4β2, has demonstrated better efficacy in reducing the smoking addiction and relapse prevention in comparison to buproprion (Jon O. Ebbert et al., Patient Preference and Adherence, 2010, 4, 355-362).

Presence of a high-affinity nicotine binding site at α4β2 nAChR, in the descending inhibitory pathways from brainstem has sparked interest in the antinociceptive properties of nicotinic ACh receptor agonists like epibatidine (Michael Decker et al., Expert Opin. Investig. Drugs, (2001), 10 (10), 1819-1830). Several new developments have opened the area for use of nicotinic modulators for therapy of pain (Michael C. Rowbotham et al., PAIN, 146, (2009), 245-252). Appropriate modulation of the nicotinic ACh receptors could provide for remedial approach to pain related states.

Another key role of the α7 nAChR is the ability to modulate the production of pro-inflammatory cytokines, like interleukins (IL), tumor necrosis factor alpha (TNF-α), and high mobility group box (HMGB-1) in the central nervous system. Consequently, an anti-inflammatory and antinociceptive effect in pain disorders have been demonstrated (M. Imad Damaj et al., Neuropharmacology, 39 (2000), 2785-2791). Additionally, ‘cholinergic anti-inflammatory pathway’ is proposed to be a regulatory of local and systemic inflammation and neuro-immune interactions through neural and humoral pathways (Margot Gallowitsch-Puerta et al., Life Sci., 2007, 80 (24-25), 2325-2329; Mauricio Rosas-Ballina et al., Mol. Med., 15 (7-8), 195-202 (2009); M. Rosas-Ballina et al., J. Intern. Med., 2009, 265, 663-679). Selective modulators of nicotinic ACh receptors, particularly α7 type, like GTS-21, attenuate cytokine production and IL-1β after endotoxin exposure. Furthermore, α7 nAChR are understood to have a central role in arthritis pathogenesis and potential therapeutic strategy for treatment of joint inflammation (M. Westman et al., Scandinavian Journal of Immunology, 2009, 70, 136-140). A putative role for α7 nAChR has also been implicated in severe sepsis, endotoxemic shock and systemic inflammation (Y. Jin, et al., International Journal of Immunogenetics, 37, 361-365; Chong Liu et al., Crit. Care Med., 2009, 37 (2), 634-641).

Angiogenesis, is a critical physiological process for the cell survival and pathologically important for cancer proliferation; several non-neural nicotinic ACh receptors, particularly α7, α5, α3, β2, β4, are involved (Hugo R. Arias et al., International Journal of Biochemistry and Cell Biology, 41 (2009), 1441-1451; Christopher Heeschen et al., The Journal of Clinical Investigation, 2002, 110 (4), 527-536). A role of nicotinic ACh receptors in the development of cervical cancer, lung carcinogenesis and pediatric lung disorders in smoking-exposed population has also been studied (Itzel E. Calleja-Macias et al., Int. J. Cancer., 124, 1090-1096 (2009); Hildegard M. Schuller et al., European Journal of Pharmacology, 393 (2000), 265-277). Several α7 nAChR agonists, partial agonists, have been characterized for their efficacy in clinical and preclinical studies. EVP-6124, an agonist at α7 nAChR, has demonstrated significant improvement in sensory processing and cognition biomarkers in Phase Ib study with patients suffering from schizophrenia (EnVivo Pharmaceuticals press release 2009, Jan. 12). GTS-21 (DMXB-Anabaseine), an α7 nAChR agonist, in the P II clinical trials, has shown efficacy in improving cognitive deficits in schizophrenia and inhibition of endotoxin-induced TNF-α release (Ann Olincy et al., Biol. Psychiatry, 2005, 57 (8, Suppl.), Abst 44; Ann Olincy et al., Arch. Gen. Psychiatry, 2006, 63, 630-638; Richard Goldstein et al., Acad. Emerg. Med., 2007, 14 (5), s185-s186). CP-810123, a α7 nAChR agonist, exhibits protection against the scopolamine-induced dementia and inhibition of amphetamine-induced auditory evoked potentials in preclinical studies (Christopher J. O'Donnell et al., J. Med. Chem., 2010, 53, 1222-1237). SSR-180711A, also an α7 nAChR agonist, enhances learning and memory, and protects against MK-801/Scopolamine-induced memory loss and prepulse inhibition in preclinical studies (John P. Redrobe et al., European Journal of Pharmacology, 602 (2009), 58-65; John Dunlop et al., Journal of Pharmacology and Experimental Therapeutics, 2009, 328, 766-776; Philippe Pichat et al., Neuropsychopharmacology, 2007, 32, 17-34). SEN-12333, protected against scopolamine-induced amnesia in passive avoidance test in preclinical studies (Renza Roncarati et al., The Journal of Pharmacology and Experimental Therapeutics, 2009, 329, 459-468). AR-R-17779, an agonist at α7 nAChR, exhibits improvement in the social recognition task performed in rats (Marja Van Kampen et al., Psychopharmacology, 2004, 172, 375-383). ABBF, an agonist at α7 nAChR, improves social recognition memory and working memory in Morris maze task in rats (Frank G. Boess et al., The Journal of Pharmacology and Experimental Therapeutics, 2007, 321, 716-725). TC-5619, a selective α7 nAChR agonist has demonstrated efficacy in animal models of positive and negative symptoms and cognitive dysfunction in schizophrenia (T. A. Hauser et al., Biochemical Pharmacology, 78 (2009), 803-812).

An alternative strategy to reinforce or potentiate the endogenous cholinergic neurotransmission of ACh without directly stimulating the target receptor is the positive allosteric modulation (PAM) of α7 nAChR (E. X. Albuquerque et al., Alzheimer Diseases and Associated Disorder, Vol. 15, Suppl 1, S19-S25). Several PAMs have been characterized, albeit in the preclinical stages of discovery. A-86774, α7 nAChR PAM, improves sensory gating in DBA/2 mice by significantly reducing the T:C ratio in a preclinical model of schizophrenia (Ramin Faghih et al., Journal of Medicinal Chemistry, 2009, 52, 3377-3384). XY-4083, an α7 nAChR PAM, normalizes the sensorimotor gating deficits in the DBA/2 mice and memory acquisition in 8-arm radial maze without altering the receptor desensitization kinetics (Herman J. Hg et al., PNAS, 2007, 104 (19), 8059-8064). Yet another PAM, PNU-120596, profoundly alters α7 nAChR desensitization kinetics and simultaneously protecting against the disruption of prepulse inhibition by MK-801. NS-1738, another PAM, has exhibited efficacy in-vivo in the animal models of social recognition and spatial memory acquisition in the Morris maze task (Daniel B. Timmermann et al., Journal of Pharmacology and Experimental Therapeutics, 2007, 323, 294-307). In addition, several patents/applications published are listed below—US 2006/0142349, US 2007/0142450, US 2009/0253691, WO 2007/031440, WO 2009/115547, WO 2009/135944, WO 2009/127678, WO 2009/127679, WO 2009/043780, WO 2009/043784, U.S. Pat. No. 7,683,084, U.S. Pat. No. 7,741,364, WO 2009/145996, US 2010/0240707, WO 2011/064288, US 2010/0222398, US 2010/0227869, EP 1866314, WO 2010/130768, WO 2011/036167, US 2010/0190819, WO 2012/104782, WO 2012/114285, WO 2012/131576, WO 2013/005153 disclose efficacy of allosteric modulators of nicotinic ACh receptors and underscoring their therapeutic potential.

### BRIEF SUMMARY OF THE INVENTION

The present invention provides compound of the general formula (I), its tautomeric forms, its stereoisomers, its pharmaceutically acceptable salts, its combinations with suitable medicament, its pharmaceutical compositions and its use as nicotinic acetylcholine receptor α7 subunit (α7 nAChR) modulator.

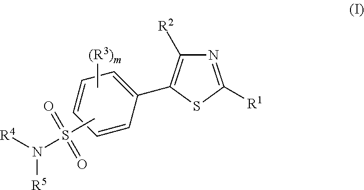

According to one aspect of the present invention there is provided compound represented by the general formula (I), its tautomeric forms, its stereoisomers, its pharmaceutically acceptable salts, its combinations with suitable medicament and its pharmaceutical compositions, wherein R¹ to R⁵ and m are described in details below.

Thus the present invention further provides a pharmaceutical composition, containing the compound of the general formula (I) as defined herein, its tautomeric forms, its stereoisomers, and its pharmaceutically acceptable salts in combination with the usual pharmaceutically employed carriers, diluents, and the like are useful for the treatment and/or prophylaxis of diseases or disorder or condition such as Alzheimer's disease (AD), mild cognitive impairment (MCI), senile dementia, vascular dementia, dementia of Parkinson's disease, attention deficit disorder, attention deficit hyperactivity disorder (ADHD), dementia associated with Lewy bodies, AIDS dementia complex (ADC), Pick's disease, dementia associated with Down's syndrome, Huntington's disease, cognitive deficits associated with traumatic brain injury (TBI), cognitive decline associated with stroke, poststroke neuroprotection, cognitive and sensorimotor gating deficits associated with schizophrenia, cognitive deficits associated with bipolar disorder, cognitive impairments associated with depression, acute pain, post-surgical or post-operative pain, chronic pain, inflammation, inflammatory pain, neuropathic pain, smoking cessation, need for new blood vessel growth associated with wound healing, need for new blood vessel growth associated with vascularization of skin grafts and lack of circulation, arthritis, rheumatoid arthritis, psoriasis, Crohn's disease, ulcerative colitis, pouchitis, inflammatory bowel disease, celiac disease, periodontitis, sarcoidosis, pancreatitis, organ transplant rejection, acute immune disease associated with organ transplantation, chronic immune disease associated with organ transplantation, septic shock, toxic shock syndrome, sepsis syndrome, depression, and rheumatoid spondylitis.

The present invention also provides a pharmaceutical composition, containing the compound of the general formula (I) as defined herein, its tautomeric forms, its stereoisomers, its pharmaceutically acceptable salts, its polymorphs, its solvates, and its optical isomers in combination with the usual pharmaceutically employed carriers, diluents, and the like are useful for the treatment and/or prophylaxis of diseases or disorder or condition classified or diagnosed as major or minor neurocognitive disorders, or disorders arising due to neurodegeneration.

The present invention also provides method of administering a compound of formula (I), as defined herein in combination with or as adjunct to medications used in the treatment of attention deficit hyperactivity disorders, schizophrenia, and other cognitive disorders such as Alzheimer's disease, Parkinson's dementia, vascular dementia or dementia associated with Lewy bodies, traumatic brain injury.

The present invention also provides method of administering a compound of formula (I), as defined herein in combination with or as an adjunct to acetylcholinesterase inhibitors, disease modifying drugs or biologics for neurodegenerative disorders, dopaminergic drugs, antidepressants, typical or an atypical antipsychotic.

The present invention also provides use of a compound of formula (I) as defined herein in the preparation of a medicament for treating a disease or disorder or condition selected from the group classified or diagnosed as major or minor neurocognitive disorders, or disorders arising due to neurodegeneration.

The present invention also provides use of a compound of formula (I) as defined herein in the preparation of a medicament for treating a disease or disorder or condition selected from attention deficit hyperactivity disorders, schizophrenia, cognitive disorders, Alzheimer's disease, Parkinson's dementia, vascular dementia or dementia associated with Lewy bodies, and traumatic brain injury.

The present invention also provides use of compound of formula (I) as defined herein in combination with or as an adjunct to acetylcholinesterase inhibitors, disease modifying drugs or biologics for neurodegenerative disorders, dopaminergic drugs, antidepressants, or a typical or atypical antipsychotic.

### DETAILED DESCRIPTION OF THE INVENTION

The present invention relates to novel compound of the general formula (I), its tautomeric forms, its stereoisomers, its pharmaceutically acceptable salts, its combinations with suitable medicament, and its pharmaceutical compositions,

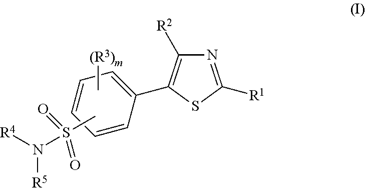

wherein,
R¹ is selected from —CR⁶(R⁷)—C(═O)—Y, —C(═O)—Z, —C(═O) NR^{6a}R^{7a}, —N(R^{6a})C(═O)R^{6b},

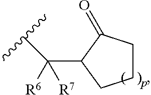

substituted- or unsubstituted-phenyl, and substituted- or unsubstituted-pyridyl; wherein substitutions on phenyl and pyridyl are selected from substituted- or unsubstituted-alkyl, halogen, and substituted- or unsubstituted-cycloalkyl;
Y is substituted- or unsubstituted-alkyl, substituted- or unsubstituted-cycloalkyl, and substituted- or unsubstituted-heterocyclyl; provided that when Y is selected as heterocyclyl the point of attachment of the said heterocyclyl is nitrogen;
Z is selected from substituted- or unsubstituted-pyridyl and substituted- or unsubstituted-cycloalkyl;
R² is selected as substituted- or unsubstituted-aryl;
R³ is selected independently at each occurrence from halogen, substituted- or unsubstituted-alkyl, perhaloalkyl, substituted- or unsubstituted-cycloalkyl, —OR^{8b}, and —C(═O)R^{8a};
R⁴ and R⁵ are independently selected from hydrogen, substituted- or unsubstituted-alkyl, and substituted- or unsubstituted-cycloalkyl;
R⁶ and R⁷ are selected from hydrogen, halogen, substituted- or unsubstituted-alkyl, and substituted- or unsubstituted-cycloalkyl; or
R⁶ and R⁷ groups and the carbon atoms to which they are attached together forming a carbocycle;
R⁶ and R^{7a} are selected from hydrogen, substituted- or unsubstituted-alkyl, and substituted- or unsubstituted-cycloalkyl;
R^{6b} is selected from substituted- or unsubstituted-alkyl, perhaloalkyl, and substituted- or unsubstituted-cycloalkyl;
R^{8a} is selected from substituted- or unsubstituted-alkyl, perhaloalkyl, and substituted- or unsubstituted-cycloalkyl;
R^{8b} is selected from hydrogen, substituted- or unsubstituted-alkyl, perhaloalkyl, and substituted- or unsubstituted-cycloalkyl;
m is an integer selected from 0, 1, and 2;
n is an integer selected from 0, 1, and 2;
p is an integer selected from 1 and 2;
wherein,
when the alkyl group is a substituted alkyl group, the alkyl group is substituted with 1 to 4 substituents selected independently from oxo, halogen, nitro, cyano, perhaloalkyl, cycloalkyl, aryl, heteroaryl, heterocyclyl, —OR^{9b}, —SO₂R^{9a}, —C(═O)OR^{9a}, —OC(═O)R^{9a}, —C(═O)N(H)R⁹, —C(═O)N(alkyl)R⁹, —N(H)C(═O)R^{9a}, —N(H)R⁹, —N(alkyl)R⁹, —N(H)C(═O)N(H)R⁹, and —N(H)C(═O)N(alkyl)R⁹;
when the cycloalkyl and the carbocycle groups are substituted, each of them is substituted with 1 to 3 substituents selected independently from oxo, halogen, nitro, cyano, alkyl, perhaloalkyl, aryl, heteroaryl, heterocyclyl, —OR^{9b}, —SO₂R^{9c}, —C(═O)R^{9c}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d};
when the aryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, —N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heteroaryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, —N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heterocyclyl group is substituted, it can be substituted either on a ring carbon atom(s) or on a ring hetero atom, when it substituted on a ring carbon atom(s), it is substituted with 1 to 3 substituents selected independently from halogen, nitro, cyano, oxo, alkyl, perhaloalkyl, cycloalkyl, aryl, heteroaryl, heterocyclyl, —OR^{9b}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d}; when the ‘heterocyclyl’ group is substituted on a ring nitrogen, it is substituted with a substituent selected from alkyl, cycloalkyl, aryl, heteroaryl, —SO₂R^{9c}, —C(═O)R^{9c}, —C(═O)N(H)R^{9d}, and —C(═O)N(alkyl)R^{9d};
R⁹ is selected from hydrogen, alkyl, cycloalkyl, aryl, heteroaryl, and heterocyclyl;
R^{9a} is selected from alkyl, perhaloalkyl, cycloalkyl, aryl, heteroaryl, and heterocyclyl;
R^{9b} is selected from hydrogen, alkyl, perhaloalkyl, cycloalkyl, aryl, heteroaryl, and heterocyclyl;
R^{9c} is selected from alkyl, perhaloalkyl, and cycloalkyl;
R^{9d} is selected from hydrogen, alkyl, and cycloalkyl.
R¹ is particularly selected from substituted- or unsubstituted-phenyl, substituted- or unsubstituted-pyridyl,

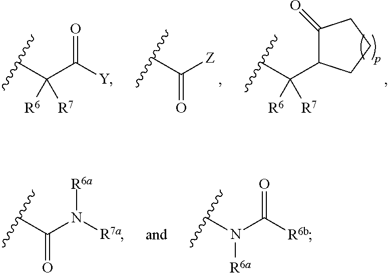

wherein, R⁶ and R⁷ are selected from hydrogen, alkyl and halogen; or R⁶ and R⁷ and the carbon to which they are attached together forming a cyclopropyl ring; Y is selected from cyclopropyl, pyrrolidine, 3-azabicyclo[3.1.0]hexane, bicyclo[3.1.0]hexane, and morpholine; Z is selected from cyclopentane and pyridyl ring; R^{6a} and R^{7a} are selected from hydrogen, substituted- or unsubstituted-alkyl, cyclopropyl, cyclopentyl, and cyclohexyl; R^{6b} is selected from substituted- or unsubstituted-alkyl, cyclopropyl, cyclopentyl, and cyclohexyl; p is an integer selected from 1 or 2.

R¹ is more particularly selected from

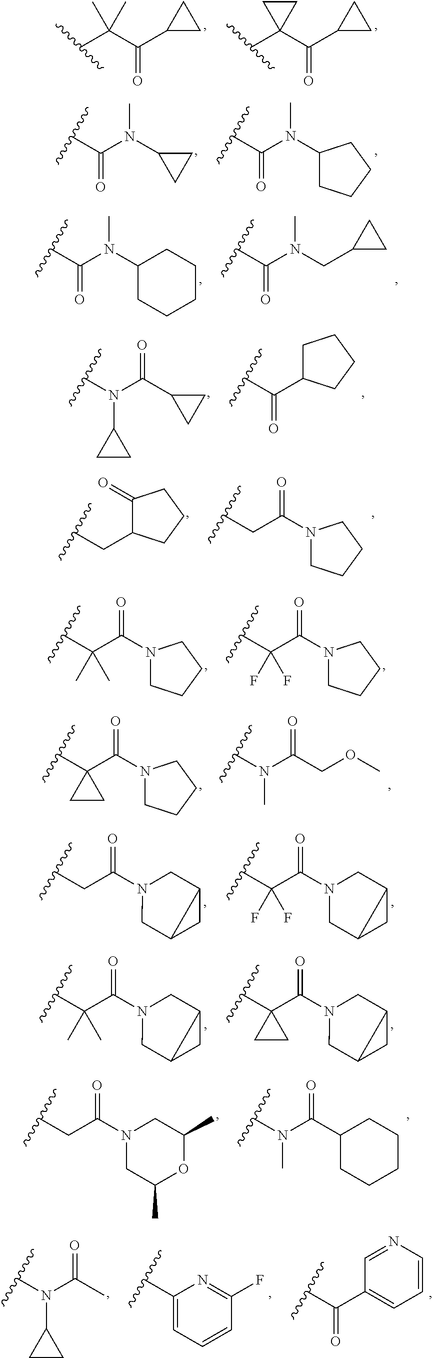

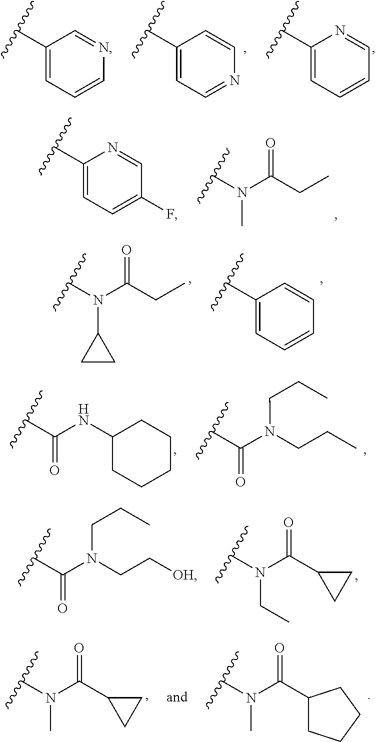

R² is particularly selected as aryl substituted with halogen.

R² is more particularly selected as phenyl substituted with chloro.

R³ is particularly selected from the halogens.

R³ is more particularly selected as fluorine.

One of the embodiments of the present invention is compound of formula (Ia)

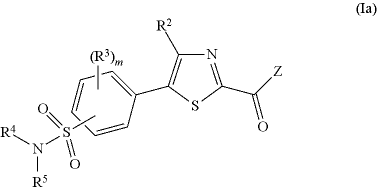

wherein, R², R³, R⁴, R⁵, m and Z are same as defined above.

Another embodiment of the present invention is compound of formula (Ib)

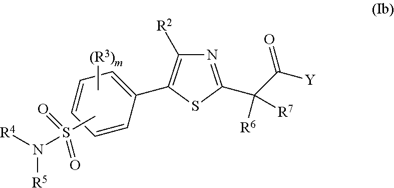

wherein, R², R³, R⁴, R⁵, R⁶, R⁷, m and Y are same as defined above.

Yet another embodiment of the present invention is compound of formula (Ic)

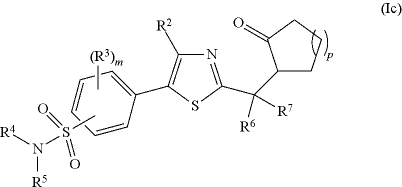

wherein, R², R³, R⁴, R⁵, R⁶, R⁷, m and p are same as defined above.

Further embodiment of the present invention is compound of formula (Id)

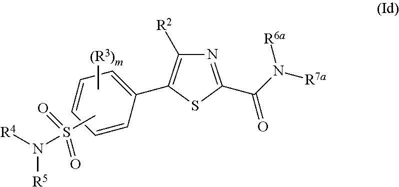

wherein, R², R³, R⁴, R⁵, R^{6a}, R^{7a} and m are same as defined above.

Yet another embodiment of the present invention is compound of formula (Ie)

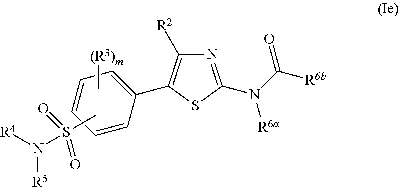

wherein, R², R³, R⁴, R⁵, R^{6a}, R^{6b} and m are same as defined above.

Whenever a range of the number of atoms in a structure is indicated (e.g., a C₁₋₁₂, C₁₋₈, C₁₋₆, or C₁₋₄ alkyl, alkylamino, etc.), it is specifically contemplated that any sub-range or individual number of carbon atoms falling within the indicated range also can be used. Thus, for instance, the recitation of a range of 1-8 carbon atoms (e.g., C₁-C₈), 1-6 carbon atoms (e.g., C₁-C₆), 1-4 carbon atoms (e.g., C₁-C₄), 1-3 carbon atoms (e.g., C₁-C₃), or 2-8 carbon atoms (e.g., C₂-C₈) as used with respect to any chemical group (e.g., alkyl, alkylamino, etc.) referenced herein encompasses and specifically describes 1, 2, 3, 4, 5, 6, 7, 8, 9, 10, 11, and/or 12 carbon atoms, as appropriate, as well as any sub-range thereof (e.g., 1-2 carbon atoms, 1-3 carbon atoms, 1-4 carbon atoms, 1-5 carbon atoms, 1-6 carbon atoms, 1-7 carbon atoms, 1-8 carbon atoms, 1-9 carbon atoms, 1-10 carbon atoms, 1-11 carbon atoms, 1-12 carbon atoms, 2-3 carbon atoms, 2-4 carbon atoms, 2-5 carbon atoms, 2-6 carbon atoms, 2-7 carbon atoms, 2-8 carbon atoms, 2-9 carbon atoms, 2-10 carbon atoms, 2-11 carbon atoms, 2-12 carbon atoms, 3-4 carbon atoms, 3-5 carbon atoms, 3-6 carbon atoms, 3-7 carbon atoms, 3-8 carbon atoms, 3-9 carbon atoms, 3-10 carbon atoms, 3-11 carbon atoms, 3-12 carbon atoms, 4-5 carbon atoms, 4-6 carbon atoms, 4-7 carbon atoms, 4-8 carbon atoms, 4-9 carbon atoms, 4-10 carbon atoms, 4-11 carbon atoms, and/or 4-12 carbon atoms, etc., as appropriate).

General terms used in formula can be defined as follows; however, the meaning stated should not be interpreted as limiting the scope of the term per se.

The term “alkyl”, as used herein, means a straight or branched hydrocarbyl chain containing from 1 to 20 carbon atoms. Preferably, the alkyl group contains 1 to 10 carbon atoms. More preferably, alkyl group contains up to 6 carbon atoms. Examples of alkyl groups include, but are not limited to, methyl, ethyl, n-propyl, iso-propyl, n-butyl, sec-butyl, iso-butyl, tert-butyl, n-pentyl, isopentyl, neopentyl, and n-hexyl.

In a substituted alkyl group, the alkyl group is substituted with 1 to 4 substituents selected independently from oxo, halogen, nitro, cyano, perhaloalkyl, cycloalkyl, aryl, heteroaryl, heterocyclyl, —OR^{9b}, —SO₂R^{9a}, —C(═O)OR^{9a}, —OC(═O)R^{9a}, —C(═O)N(H)R⁹, —C(═O)N(alkyl)R⁹, —N(H)C(═O)R^{9a}, —N(H)R⁹, —N(alkyl)R⁹, —N(H)C(═O)N(H)R⁹, and —N(H)C(═O)N(alkyl)R⁹; wherein R is selected from hydrogen, alkyl, cycloalkyl, aryl, heteroaryl, and heterocyclyl; R^{9a} is selected from alkyl, perhaloalkyl, cycloalkyl, aryl, heteroaryl, and heterocyclyl; R^{9b} is selected from hydrogen, alkyl, perhaloalkyl, cycloalkyl, aryl, heteroaryl, and heterocyclyl.

The term “perhaloalkyl” used herein means an alkyl group as defined hereinabove wherein all the hydrogen atoms of the said alkyl group are substituted with halogen. The perhaloalkyl group is exemplified by trifluoromethyl, pentafluoroethyl, and the like.

The term “cycloalkyl” as used herein, means a monocyclic, bicyclic, or tricyclic non-aromatic ring system containing from 3 to 14 carbon atoms, preferably monocyclic cycloalkyl ring containing 3 to 6 carbon atoms. Examples of monocyclic ring systems include cyclopropyl, cyclobutyl, cyclopentyl, cyclohexyl, cycloheptyl, and cyclooctyl. Bicyclic ring systems include monocyclic ring system fused across a bond with another cyclic system which may be an alicyclic ring or an aromatic ring. Bicyclic rings also include spirocyclic systems wherein the second ring gets annulated on a single carbon atom. Bicyclic ring systems are also exemplified by a bridged monocyclic ring system in which two non-adjacent carbon atoms of the monocyclic ring are linked by an alkylene bridge. Examples of bicyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, bicyclo[3.3.2]decane, bicyclo[3.1.0]hexane, bicyclo[410]heptane, bicyclo[3.2.0]heptanes, octahydro-1H-indene, spiro[2.5]octane, spiro[4.5]decane, spiro[bicyclo[4.1.0]heptane-2,1′-cyclopentane], hexahydro-2′H-spiro[cyclopropane-1,1′-pentalene]. Tricyclic ring systems are the systems wherein the bicyclic systems as described about are further annulated with third ring, which may be alicyclic ring or aromatic ring. Tricyclic ring systems are also exemplified by a bicyclic ring system in which two non-adjacent carbon atoms of the bicyclic ring are linked by a bond or an alkylene bridge. Examples of tricyclic-ring systems include, but are not limited to, tricyclo[3.3.1.0^{3.7}]nonane and tricyclo[3.3.1.1^{3.7}]decane (adamantane).

The term “carbocycle” as used herein, means a cyclic system made up of carbon atoms, which includes cycloalkyl, and aryl.

When the cycloalkyl or the carbocycle groups are substituted, they are substituted with 1 to 3 substituents selected independently from oxo, halogen, nitro, cyano, alkyl, perhaloalkyl, aryl, heteroaryl, heterocyclyl, —OR^{9b}, —SO₂R^{9c}, —C(═O)R^{9c}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d}; wherein R^{9b} is selected from hydrogen, alkyl, perhaloalkyl, cycloalkyl, aryl, heteroaryl, and heterocyclyl; R^{9c} is selected from alkyl, perhaloalkyl, and cycloalkyl; R^{9d} is selected from hydrogen, alkyl, and cycloalkyl.

The term “aryl” refers to a monocyclic, bicyclic or tricyclic aromatic hydrocarbon ring system. Examples of aryl groups include phenyl, naphthyl, anthracenyl, fluorenyl, indenyl, azulenyl, and the like. Aryl group also includes partially saturated bicyclic and tricyclic aromatic hydrocarbons such as tetrahydro-naphthalene.

When the aryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, —N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂.

The term “heteroaryl” refers to a 5-14 membered monocyclic, bicyclic, or tricyclic ring system having 1 to 4 ring heteroatoms selected from O, N, or S, and the remainder ring atoms being carbon (with appropriate hydrogen atoms unless otherwise indicated), wherein at least one ring in the ring system is aromatic. Heteroaryl groups may be optionally substituted with one or more substituents. In one embodiment, 0, 1, 2, 3, or 4 atoms of each ring of a heteroaryl group may be substituted by a substituent. Examples of heteroaryl groups include, but not limited to pyridyl, 1-oxo-pyridyl, furanyl, thienyl, pyrrolyl, oxazolyl, oxadiazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, benzoxazolyl, benzofuranyl, indolizinyl, imidazopyridyl, tetrazolyl, benzimidazolyl, benzothiazolyl, benzothiadiazolyl, benzoxadiazolyl, indolyl, azaindolyl, imidazopyridyl, quinazolinyl, purinyl, pyrrolo[2,3]pyrimidinyl, pyrazolo[3,4]pyrimidinyl, and benzo(b)thienyl, 2,3-thiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c]isoxazolyl, 2,3-dihydro-benzo[1,4]dioxin-6-yl, 2,3-dihydro-benzo[1,4]dioxin-5-yl, 2,3-dihydro-benzofuran-5-yl, 2,3-dihydro-benzofuran-4-yl, 2,3-dihydro-benzofuran-6-yl, 2,3-dihydro-benzofuran-6-yl, 2,3-dihydro-1H-indol-5-yl, 2,3-dihydro-1H-indol-4-yl, 2,3-dihydro-1H-indol-6-yl, 2,3-dihydro-1H-indol-7-yl, benzo[1,3]dioxol-4-yl, benzo[1,3]dioxol-5-yl, 1,2,3,4-tetrahydroquinolinyl, 1,2,3,4-tetrahydroisoquinolinyl, 2,3-dihydrobenzothien-4-yl, 2-oxoindolin-5-yl and the like.

When the heteroaryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, —N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂.

The term “heterocyclyl” as used herein, means a ‘cycloalkyl’ group wherein one or more of the carbon atoms replaced by —O—, —S—, —S(O₂)—, —S(O)—, —N(R^{m})—, —Si(R^{m})Rⁿ—, wherein, R^{m} and Rⁿ are independently selected from hydrogen, alkyl, aryl, heteroaryl, cycloalkyl, and heterocyclyl. The heterocycle may be connected to the parent molecular moiety through any carbon atom or any nitrogen atom contained within the heterocycle. Examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1.1-dioxidothiomorpholinyl(thiomorpholine sulfone), thiopyranyl, and trithianyl. Examples of bicyclic heterocycle include, but are not limited to 1,3-benzodioxolyl, 1,3-benzodithiolyl, 2,3-dihydro-1,4-benzodioxinyl, 2,3-dihydro-1-benzofuranyl, 2,3-dihydro-1-benzothienyl, 2,3-dihydro-1H-indolyl and 1,2,3,4-tetrahydroquinolinyl. The term heterocycle also include bridged heterocyclyl systems such as azabicyclo[3.2.1]octane, azabicyclo[3.3.1]nonane and the like.

The heterocyclyl group, when it is substituted, it may be substituted on ring carbon atom or ring nitrogen atom. For example, it is substituted on ring carbons with 1 to 3 substituents selected independently from halogen, nitro, cyano, oxo, alkyl, perhaloalkyl, cycloalkyl, aryl, heteroaryl, heterocyclyl, —OR^{9b}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d}; wherein R^{9b} is selected from hydrogen, alkyl, perhaloalkyl, cycloalkyl, aryl, heteroaryl, and heterocyclyl; R^{9c} is selected from alkyl, perhaloalkyl, and cycloalkyl; R^{9d} is selected from hydrogen, alkyl, and cycloalkyl.

When the heterocyclyl group is substituted on ring nitrogen(s), it is substituted with a substituent selected from alkyl, cycloalkyl, aryl, heteroaryl, —SO₂R^{9c}, —C(═O)R^{9c}, —C(═O)N(H)R^{9d}, and —C(═O)N(alkyl)R^{9d}; wherein R^{9c} is selected from alkyl, perhaloalkyl, and cycloalkyl; R^{9d} is selected from hydrogen, alkyl, and cycloalkyl.

When a parent group is substituted with an “oxo” group, it means a divalent oxygen (═O) becomes attached to a carbon atom of the parent group. For example, when a CH₂ group is substituted with an oxo substituent, the parent CH₂ group becomes a carbonyl (C═O) group; thus, oxo substituted on cyclohexane forms a cyclohexanone, for example.

The term “annulated” means the ring system under consideration is either annulated with another ring at a carbon atom of the cyclic system or across a bond of the cyclic system as in the case of fused or spiro ring systems.

The term “bridged” means the ring system under consideration contain an alkylene bridge having 1 to 4 methylene units joining two non-adjacent ring atoms.

A compound, its stereoisomers, racemates, and pharmaceutically acceptable salt thereof as described hereinabove, wherein, the compound of general formula (I) is selected from:
1. 4-(4-(4-chlorophenyl)-2-(cyclopentanecarbonyl)thiazol-5-yl)benzenesulfonamide (compound 1);
2. 4-(4-(4-chlorophenyl)-2-(cyclopentanecarbonyl)thiazol-5-yl)-3-fluorobenzenesulfonamide (compound 2);
3. 4-(4-(4-chlorophenyl)-2-picolinoylthiazol-5-yl)benzenesulfonamide (compound 3);
4. 4-(4-(4-chlorophenyl)-2-nicotinoylthiazol-5-yl)benzenesulfonamide (compound 4);
5. 4-(4-(4-chlorophenyl)-2-(1-cyclopropyl-2-methyl-1-oxopropan-2-yl)thiazol-5-yl)-3-fluorobenzenesulfonamide (compound 5);
6. 4-(4-(4-chlorophenyl)-2-(1-cyclopropyl-2-methyl-1-oxopropan-2-yl)thiazol-5-yl)benzenesulfonamide (compound 6);
7. 4-(4-(4-chlorophenyl)-2-(1-(cyclopropanecarbonyl)cyclopropyl)thiazol-5-yl)-3-fluorobenzenesulfonamide (compound 7);
8. 4-(4-(4-chlorophenyl)-2-((2-oxocyclopentyl)methyl)thiazol-5-yl)benzenesulfonamide (compound 8);
9. 4-(4-(4-chlorophenyl)-2-((2-oxocyclopentyl)methyl)thiazol-5-yl)-3-fluorobenzenesulfonamide (compound 9);
10. 4-(2-(2-(3-azabicyclo[3.1.0]hexan-3-yl)-2-oxoethyl)-4-(4-chlorophenyl)thiazol-5-yl)benzenesulfonamide (compound 10);
11. (cis)-4-(4-(4-chlorophenyl)-2-(2-(2,6-dimethylmorpholino)-2-oxoethyl)thiazol-5-yl)benzenesulfonamide (compound 11);
12. 4-(4-(4-chlorophenyl)-2-(2-oxo-2-(pyrrolidin-1-yl)ethyl)thiazol-5-yl)benzenesulfonamide (compound 12);
13. 4-(2-(1-(3-azabicyclo[3.1.0]hexan-3-yl)-2-methyl-1-oxopropan-2-yl)-4-(4-chlorophenyl)thiazol-5-yl)benzenesulfonamide (compound 13);
14. 4-(4-(4-chlorophenyl)-2-(2-methyl-1-oxo-1-(pyrrolidin-1-yl)propan-2-yl)thiazol-5-yl)benzenesulfonamide (compound 14);
15. 4-(2-(1-(3-azabicyclo[3.1.0]hexane-3-carbonyl)cyclopropyl)-4-(4-chlorophenyl)thiazol-5-yl)benzenesulfonamide (compound 15);
16. 4-(4-(4-chlorophenyl)-2-(1-(pyrrolidine-1-carbonyl)cyclopropyl)thiazol-5-yl)benzenesulfonamide (compound 16);
17. 4-(2-(2-(3-azabicyclo[3.1.0]hexan-3-yl)-1,1-difluoro-2-oxoethyl)-4-(4-chlorophenyl)thiazol-5-yl)benzenesulfonamide (compound 17);
18. 4-(4-(4-chlorophenyl)-2-(1,1-difluoro-2-oxo-2-(pyrrolidin-1-yl)ethyl)thiazol-5-yl)benzenesulfonamide (compound 18);
19. 4-(4-(4-chlorophenyl)-2-(6-fluoropyridin-2-yl)thiazol-5-yl)benzenesulfonamide (compound 19);
20. 4-(4-(4-chlorophenyl)-2-(pyridin-3-yl)thiazol-5-yl)benzenesulfonamide (compound 20);
21. 4-(4-(4-chlorophenyl)-2-(pyridin-4-yl)thiazol-5-yl)benzenesulfonamide (compound 21);
22. 4-(4-(4-chlorophenyl)-2-(pyridin-2-yl)thiazol-5-yl)benzenesulfonamide (compound 22);
23. 4-(4-(4-chlorophenyl)-2-(pyridin-2-yl)thiazol-5-yl)-3-fluorobenzenesulfonamide (compound 23);
24. 4-(4-(4-chlorophenyl)-2-phenylthiazol-5-yl)benzenesulfonamide (compound 24);
25. 4-(4-(4-chlorophenyl)-2-(5-fluoropyridin-2-yl)thiazol-5-yl)benzenesulfonamide (compound 25);
26. 4-(4-chlorophenyl)-N-cyclopropyl-N-methyl-5-(4-sulfamoylphenyl)thiazole-2-carboxamide (compound 26);
27. 4-(4-chlorophenyl)-N-cyclopropyl-5-(2-fluoro-4-sulfamoylphenyl)-N-methylthiazole-2-carboxamide (compound 27);
28. 4-(4-chlorophenyl)-N-cyclopentyl-N-methyl-5-(4-sulfamoylphenyl)thiazole-2-carboxamide (compound 28);
29. 4-(4-chlorophenyl)-N-cyclohexyl-N-methyl-5-(4-sulfamoylphenyl)thiazole-2-carboxamide (compound 29);
30. 44-(4-chlorophenyl)-N-(cyclopropylmethyl)-N-methyl-5-(4-sulfamoylphenyl)thiazole-2-carboxamide (compound 30);
31. 4-(4-chlorophenyl)-N-cyclohexyl-5-(4-sulfamoylphenyl)thiazole-2-carboxamide (compound 31);
32. 4-(4-chlorophenyl)-N,N-dipropyl-5-(4-sulfamoylphenyl)thiazole-2-carboxamide (compound 32);
33. 4-(4-chlorophenyl)-N-(2-hydroxyethyl)-N-propyl-5-(4-sulfamoylphenyl)thiazole-2-carboxamide (compound 33);
34. N-(4-(4-chlorophenyl)-5-(4-sulfamoylphenyl)thiazol-2-yl)-N-ethylcyclopropanecarboxamide (compound 34);
35. N-(4-(4-chlorophenyl)-5-(4-sulfamoylphenyl)thiazol-2-yl)-N-methylcyclopropanecarboxamide (compound 35);
36. N-(4-(4-chlorophenyl)-5-(4-sulfamoylphenyl)thiazol-2-yl)-N-methylcyclopentanecarboxamide (compound 36);
37. N-(4-(4-chlorophenyl)-5-(4-sulfamoylphenyl)thiazol-2-yl)-N-methylcyclohexanecarboxamide (compound 37);
38. N-(4-(4-chlorophenyl)-5-(4-sulfamoylphenyl)thiazol-2-yl)-N-methylpropionamide (compound 38);
39. N-(4-(4-chlorophenyl)-5-(4-sulfamoylphenyl)thiazol-2-yl)-2-methoxy-N-methylacetamide (compound 39);
40. N-(4-(4-chlorophenyl)-5-(4-sulfamoylphenyl)thiazol-2-yl)-N-cyclopropylacetamide (compound 40);
41. N-(4-(4-chlorophenyl)-5-(4-sulfamoylphenyl)thiazol-2-yl)-N-cyclopropylpropionamide (compound 41); and
42. N-(4-(4-chlorophenyl)-5-(4-sulfamoylphenyl)thiazol-2-yl)-N-cyclopropylcyclopropanecarboxamide (compound 42).

According to another aspect of the present invention, the compound of general formula (I) where all the symbols are as defined earlier was prepared by methods described below. However, the synthetic methods should not be construed limiting the invention, which lies in the whole genus described by compound of formula (I) above.

Scheme 1 below shows a method of preparation of the compound of the formula (Ia), where R², R³, R⁴, R⁵, Z, and m are as described under the compound of formula (I), from compound represented by general formula (II), where R² is same as defined under the compound of formula (I).

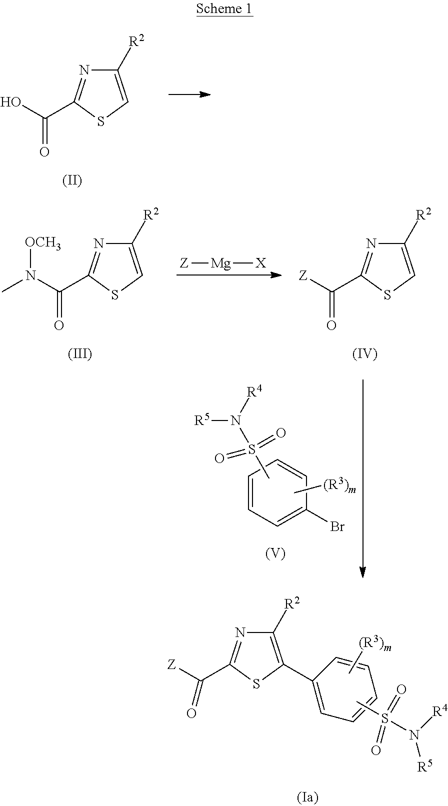

The compound of formula (II) can be prepared by following the procedure described in US 2007/32531. Compound (II) is reacted with N,O-dimethylhydroxylamine hydrochloride to obtain the compound of the formula (III). The said reaction can be carried out using the conditions generally utilized for converting carboxylic acids to amides. The reaction can be carried out in the presence of an organic solvent, for example, DMF, THF, a halogenated hydrocarbon such as chloroform and dichloromethane, an aromatic hydrocarbon such as xylene, benzene, toluene, or mixtures thereof or the like in the presence of suitable base such as triethylamine, diisopropylethylamine, pyridine or the like at a temperature between 0-50° C. using reagents such as 1-(3-dimethylaminopropyl)-3-ethylcarbodimide hydrochloride (EDCI), 1,3-dicyclohexylcarbodiimide (DCC), and auxiliary reagents such as 1-hydroxy-7-azabenzotriazole (HOAT), hydroxybenzotriazole hydrate (HOBT) or the like. Preferably, the reaction is carried out in DMF using EDC, HOBT and triethylamine as base.

Compound of the formula (III) so obtained is reacted with a Grignard reagent Z—Mg—X, where Z is as defined under generic formula (I), and X is a halogen, to obtain the compound of formula (IV). The reaction of compound of formula (III) with Z—Mg—X can be carried out according to the procedure given by Ramin Faghih et al., Journal of Medicinal Chemistry, 2009, 52, 3377-3384. Preferably, the reaction is carried out in THF at room temperature.

The compound of the formula (IV) as obtained is then reacted with bromobenzene sulphonamide of formula (V), where R³, R⁴, R⁵ and m are as defined under the compound of formula (I), to obtain the compound of formula (Ia). The said reaction can be carried out following the procedures provided by Julien Roger et al., Adv. Synth. Catal., 2009, 351, 1977-1990. The reaction can be carried out in an organic solvent such as N,N-dimethylformamide, N,N-dimethylacetamide, toluene, or the like in presence of potassium carbonate, potassium phosphate or potassium acetate, and a palladium catalyst. The inventors have carried out the coupling reaction in dimethyl acetamide in the presence of potassium acetate and palladium acetate at a temperature of about 150° C.

Scheme 2 below shows a method of preparation of the compound of the formula (Ib), wherein R⁶, R⁷ are substituted- or unsubstituted-alkyl, and substituted- or unsubstituted-cycloalkyl, or R⁶ and R⁷ groups and the carbon atoms to which they are attached together form carbocycle, Y is substituted- or unsubstituted-alkyl, and substituted- or unsubstituted-cycloalkyl, R², R³, R⁴, R⁵, and m are as described under the compound of formula (I), from compound represented by general formula (VI), where R² is as defined under general formula (I).

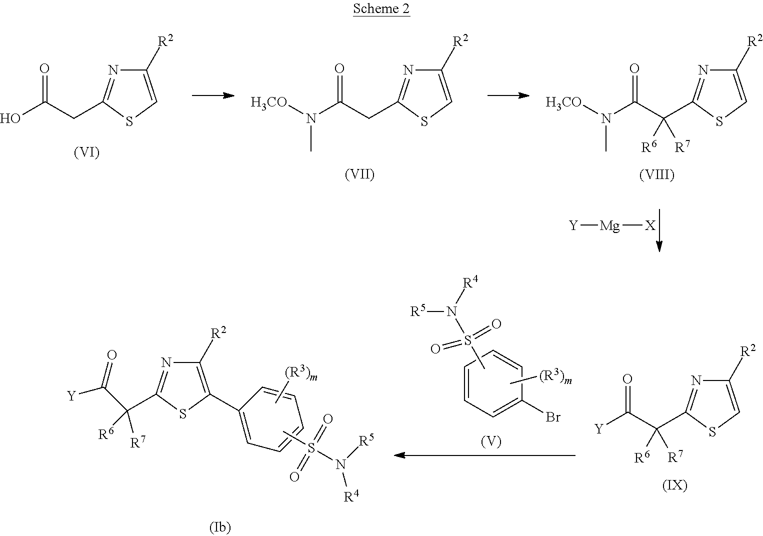

The compound of formula (VI) (Prepared according to the procedure described in US 2007/32531) is reacted with N, O-dimethylhydroxylamine hydrochloride to obtain the compound of the formula (VII). The reaction can be carried out under the conditions as described earlier in Scheme 1 for the conversion of compound of formula (II) to (III).

The compound of formula (VII) as obtained is then reacted with alkyl/cycloalkyl/alkylene halides in presence of base like sodium ethoxide, NaH, potassium t-butoxide to obtain the compound of formula (VIII), where R⁶ and R⁷ are substituted- or unsubstituted-alkyl, and substituted- or unsubstituted-cycloalkyl, or R⁶ and R⁷ groups and the carbon atoms to which they are attached together form carbocycle. Preferably, the said alkylation reaction is carried out in DMF in presence of sodium methoxide.

Compound of the formula (VIII) so obtained is reacted with a Grignard reagent Y—Mg—X (where Y is substituted- or unsubstituted-alkyl, and substituted- or unsubstituted-cycloalkyl, and X is a halogen) to obtain the compound of formula (IX), where Y, R⁶, R⁷ and R² are as defined earlier. The reaction can be carried out under conditions as described earlier in Scheme 1 for the conversion of compound of formula (III) to (IV).

The compound of the formula (IX) as obtained is then reacted with bromobenzene sulphonamide of formula (V) to obtain the compound of formula (Ib), wherein R², R³, R⁴, R⁵, R⁶, R⁷, Y, and m are as defined under the compound of formula (I). The reaction can be carried out under the conditions as described earlier in Scheme 1 for conversion of compound of formula (IV) to (Ia).

Scheme 3 below shows a method of preparation of the compound of the formula (Ic), wherein R⁶, R⁷ are hydrogens; R², R³, R⁴, R⁵, m and p are as described under compound of formula (I), from compound represented by general formula (X), where R² is same as defined under general formula (I).

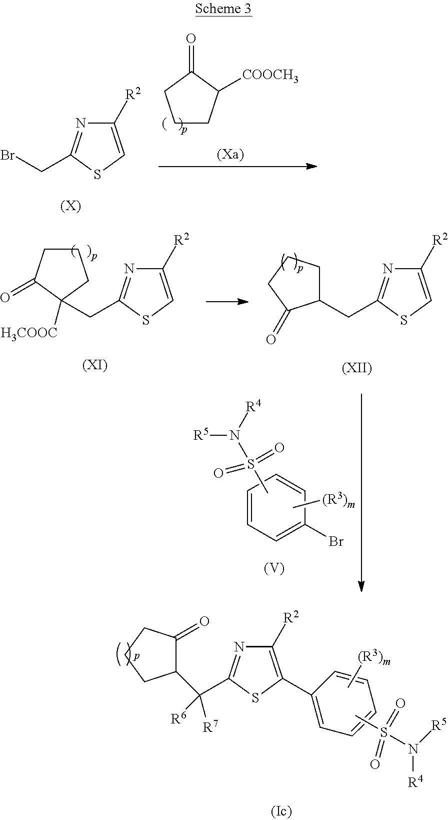

The compound of formula (X) (prepared according to the procedure described in WO 2007/107758) is reacted with compound of formula (Xa) in presence of base such as potassium carbonate, sodium ethoxide, NaH, potassium t-butoxide and in an organic solvent such as DMF, THF and acetone to obtain the compound of the formula (XI). Preferably, the said reaction is carried out in acetone in presence of potassium carbonate.

The compound of formula (XI) so obtained is subjected to de-carboxylation reaction to obtain the compound of formula (XII). The reaction may be carried out in the presence of suitable acid such as acetic acid, HCl, H₂SO₄ or the like at a temperature between 0-200° C. Preferably, the de-carboxylation reaction is carried out in acetic acid and HCl at a temperature 105° C.

The compound of the formula (XII) so obtained is then reacted with bromobenzene sulphonamide of formula (V) to obtain the compound of formula (Ic). The said reaction can be carried out under the conditions as described earlier in Scheme 1 for the conversion of compound of formula (IV) to (Ia).

Scheme 4 shows route of synthesis of the compound of the formula (Ib), where R⁶, R⁷ are fluoro, Y is heterocyclyl, R², R³, R⁴, R⁵ and m are as described under compound of formula (I), from 4-bromo-2-(trimethylsilyl)thiazole (XIII).

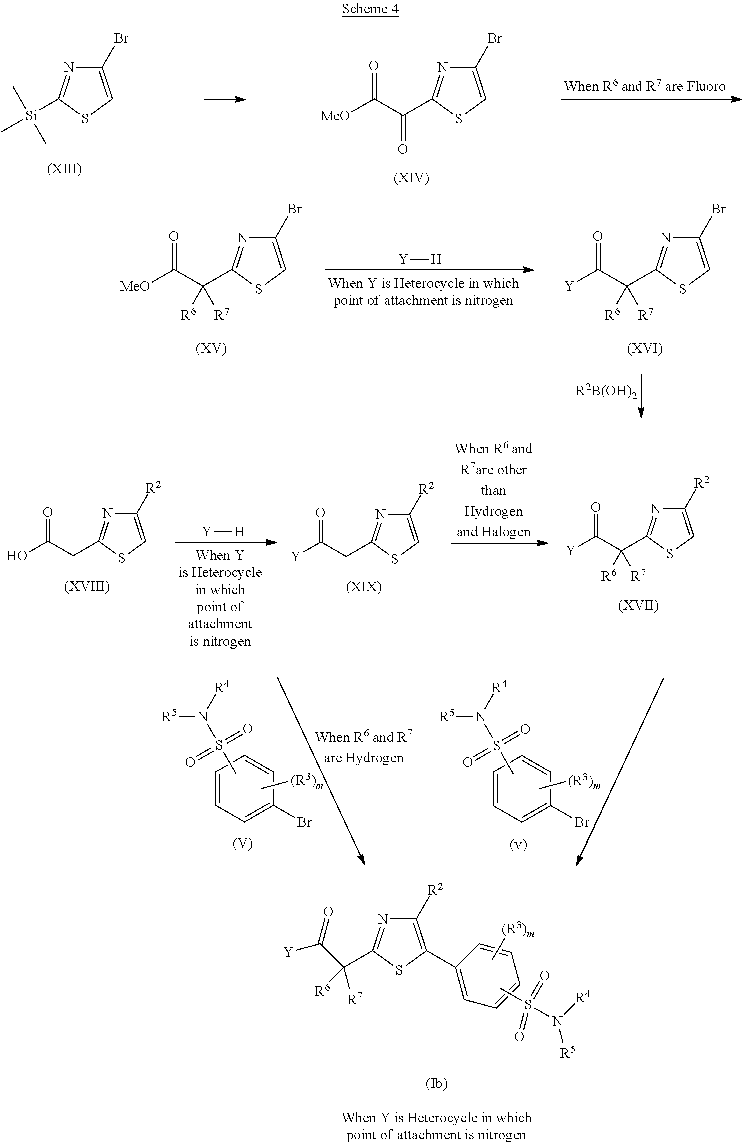

Compound of formula (XIII) (Prepared according to the procedure described by A. Dondoni et al., Synthesis, 1986, 9, 757-760) is reacted with methyl oxalyl chloride to obtain compound of formula (XIV). The compound of formula (XIV) is then subjected to fluorination reaction in presence of diethyl amino sulfur trifluoride (DAST) to obtain compound of formula (XV) (where R⁶ and R⁷ are fluoro).

Compound of formula (XV) is then reacted with appropriate amine (Y-H) to obtain compound of formula (XVI), where R⁶ and R⁷ are fluoro and Y is heterocyclyl. The reaction can be carried out in the presence of an organic solvent, for example DMF, THF, a halogenated hydrocarbon such as chloroform and dichloromethane, an aromatic hydrocarbon such as xylene, benzene, toluene, or mixtures thereof or the like, and a suitable base such as potassium carbonate, triethylamine, diisopropylethylamine, pyridine or the like at temperature about 25° C. or higher. Preferably, the coupling reaction is carried out in dichloromethane in presence of triethylamine at 25° C.

Compound of formula (XVI) is then subjected to Suzuki coupling with R²B(OH)₂ to obtain the compound of formula (XVII), where R⁶, R⁷ are fluoro, Y is heterocyclyl and R² is same as defined under the general formula (I). The reaction can be carried out with boronic acids and esters using the reaction conditions well known in the art. Preferably, the reaction is carried out in a mixture of ethanol and toluene, in presence of base such as potassium phosphate, potassium carbonate or the like, and tetrakis(triphenylphosphine) palladium(0) at a temperature of about 50° C. or higher. Boronic acid used in this reaction can be prepared by the methods well known in the art by hydrolyzing the corresponding boronate. Boronates are generally commercially available, besides, such boronates can also be prepared by reacting an appropriate iodo or bromo compound with an alkyl lithium such as butyl lithium and then reacting with a borate ester or by methods as described in EP1012142.

Alternatively, the compound of formula (XVII), where R², R⁶ and R⁷ are as defined under the compound of formula (I) and Y is heterocyclyl, was synthesized from compound of formula (XVIII), where R² is as defined under the compound of formula (I). Compound of formula (XVIII) (Prepared according to the procedure described in literature US 2007/32531) is reacted with an appropriate amine (Y-H) to obtain compound of formula (XIX). The reaction can be carried out according to the conditions generally used for converting carboxylic acids to amides. The reaction may be carried out in an organic solvent such as DMF, THF, a halogenated hydrocarbon such as chloroform and dichloromethane, an aromatic hydrocarbon such as xylene, benzene, toluene, or mixtures thereof or the like in the presence of suitable base such as triethylamine, diisopropylethylamine, pyridine or the like at a temperature between 0-50° C. using reagents such as 1-(3-dimethylaminopropyl)-3-ethylcarbodimide hydrochloride (EDCI), 1,3-dicyclohexylcarbodiimide (DCC), and auxiliary reagents such as 1-hydroxy-7-azabenzotriazole (HOAT), hydroxybenzotriazole hydrate (HOBT) or the like. Preferably, the reaction is carried out in DMF using EDC, HOBT and triethylamine as base.

The compound of formula (XIX) is further reacted with alkyl/cycloalkyl/alkylene halides in presence of base like sodium ethoxide, sodium hydride, Potassium t-butoxide to obtain the compound of formula (XVII), where R², R⁶ and R⁷ are same as defined under the general formula (I) and Y is heterocyclyl. Preferably, the alkylation reaction is carried out in DMF in presence of sodium ethoxide.

The compound of formula (XVII) is further reacted with bromobenzenesulphonamide of formula (V) to obtain compound of the formula (Ib), where R⁶, R⁷ are halogen, substituted- or unsubstituted-alkyl, and substituted- or unsubstituted-cycloalkyl, or R⁶ and R⁷ groups and the carbon atoms to which they are attached together forming a carbocycle, Y is heterocyclyl, R², R³, R⁴, R⁵, and m are the same as defined under the compound of formula (I). The reaction can be carried out in an organic solvent such as N,N-dimethylformamide, N,N-dimethylacetamide, toluene, or the like in presence of base such as potassium carbonate, potassium phosphate or potassium acetate, and a palladium catalyst. The inventors have carried out the said reaction in dimethyl acetamide in presence of potassium acetate and palladium acetate at a temperature of about 150° C.

Alternatively, the compound of the formula (XIX) is reacted with bromobenzenesulphonamide of formula (V) to obtain the compound of formula (Ib), where R⁶, R⁷ are hydrogen, Y is heterocyclyl, R², R³, R⁴, R⁵, and m are the same as defined under the compound of formula (I), using the reaction conditions described in the paragraph above.

Scheme 5 below shows route of synthesis of the compound of the formula (I), where R¹ is substituted- or unsubstituted-phenyl, and substituted- or unsubstituted-pyridyl; R², R³, R⁴, R⁵ and m as described under the compound of formula (I), starting from 2,4-dibromothiazole (XX).

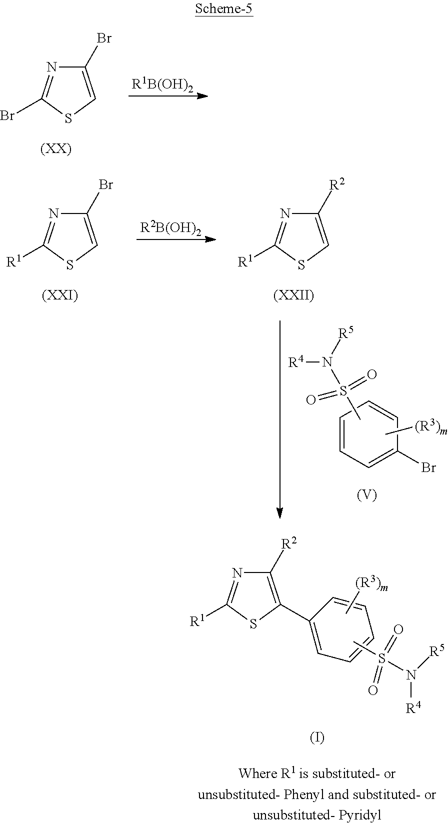

2,4-dibromothiazole (XX), which is commercially available, is subjected to Suzuki coupling with R¹B(OH)₂ to obtain the compound of formula (XXI), where R¹ is substituted- or unsubstituted-phenyl and substituted- or unsubstituted-pyridyl. Suzuki coupling can be carried out under the conditions as described earlier in Scheme 4 for the conversion of compound of formula (XVI) to (XVII).

Compound of formula (XXI) then reacted with R²B(OH)₂ to obtain the compound of formula (XXII), where R¹ is substituted- or unsubstituted-phenyl and substituted- or unsubstituted-pyridyl, R² is same as defined under the compound of formula (I). The Suzuki coupling reaction was carried out using the reaction conditions described in the paragraph above.

The compound of formula (XXII) is further reacted with bromobenzene sulphonamide of formula (V), where R³, R⁴, R⁵ and m are as defined under the compound of formula (I), to obtain compound of the formula (I), where R¹ is substituted- or unsubstituted-phenyl and substituted- or unsubstituted-pyridyl; R², R³, R⁴, R⁵ and m as described under compound of formula (I). The reaction can be carried out in an organic solvent such as N,N-dimethylformamide, N,N-dimethylacetamide, toluene, or the like in presence of potassium carbonate, potassium phosphate or potassium acetate, and a palladium catalyst. The inventors have carried out the reaction in dimethyl acetamide in presence of potassium acetate and palladium acetate at a temperature of about 150° C.

Scheme 6 shows route of synthesis of the compound of the formula (Id), where R², R³, R⁴, R⁵, R^{6a}, R^{7a} and m as described under compound of formula (I), from compound represented by general formula (XXIII), where R² and R^{6a} are same as defined under the compound of formula (I).

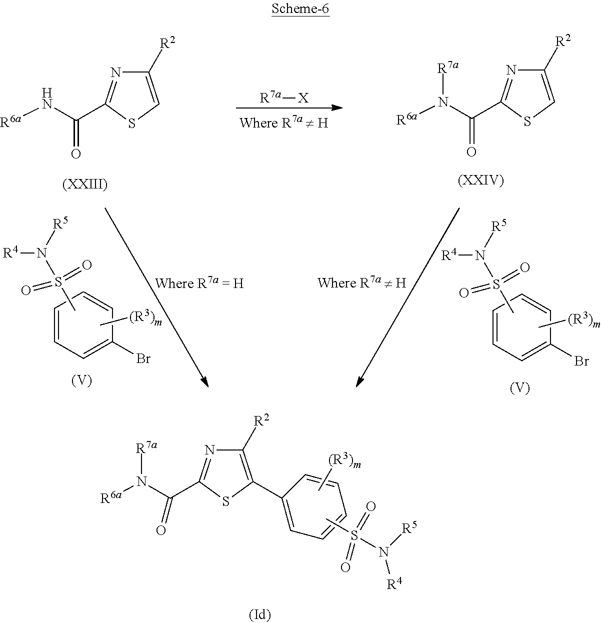

Compound of formula (XXIII) (Prepared according to the procedure described in literature US 2009/318429) is reacted with alkyl/cycloalkyl halides in presence of base such as sodium ethoxide, sodium hydride, Potassium t-butoxide to obtain the compound of formula (XXIV), where R^{7a} is selected from substituted- or unsubstituted-alkyl, and substituted- or unsubstituted-cycloalkyl, R² and R^{6a} are same as defined under the compound of formula (I). Preferably, the alkylation reaction is carried out in DMF in presence of sodium hydride.

The compound of formula (XXIV) is further reacted with bromobenzenesulphonamide of formula (V), where R³, R⁴, R⁵ and m are as defined in compound of formula (I), to obtain compound of the formula (Id), where R^{7a} is selected from substituted- or unsubstituted-alkyl, and substituted- or unsubstituted-cycloalkyl, R², R³, R⁴, R⁵, R^{6a}, and m are the same as defined compound of formula (I). The reaction can be carried out in an organic solvent such as N,N-dimethylformamide, N,N-dimethylacetamide, toluene, or the like in presence of base such as potassium carbonate, potassium phosphate or potassium acetate, and a palladium catalyst. The inventors have carried out the reaction in dimethyl acetamide in presence of potassium acetate and palladium acetate at a temperature of about 150° C.

Alternatively, the compound of the formula (XXIII) is reacted with bromobenzenesulphonamide of formula (V) to obtain the compound of formula (Id), where R^{7a} is Hydrogen, R², R³, R⁴, R⁵, R^{6a}, and m are the same as defined under the compound of formula (I), using the reaction conditions described in the paragraph above.

Scheme 7 shows route for synthesis of the compound of the formula (Ie), where R^{6a} is selected from substituted- or unsubstituted-alkyl, R², R³, R⁴, R⁵, R^{6b}, and m as described under compound of formula (I), from compound of the formula (XXV), where R² is same as defined under the compound of formula (I).

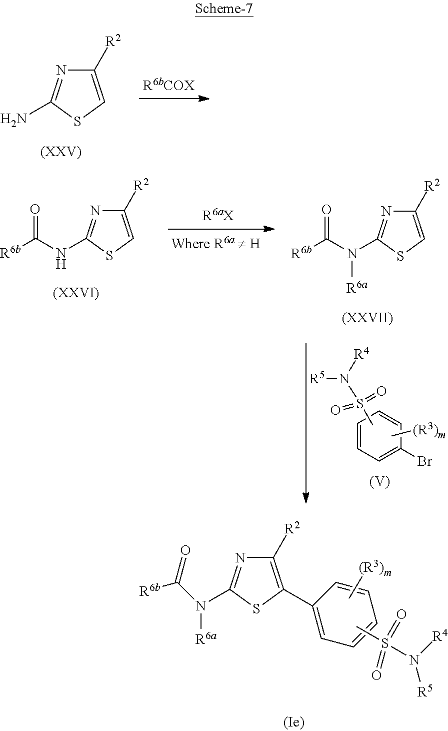

Compound of formula (XXV) (Prepared according to the procedure described by G. L. Talesara et al., Journal of Indian chemical society, 2008, 85, 660-664) is reacted with R^{6b}COX, where R^{6b} is as defined under the compound of formula (I), to obtain compound of formula (XXVI), where R² and R^{6b} are as described under the compound of formula (I). The reaction can be carried out in the presence of an organic solvent such as dimethyl acetamide, DMF, THF, a halogenated hydrocarbon such as chloroform and dichloromethane, an aromatic hydrocarbon such as xylene, benzene, toluene, or mixtures thereof or the like, and a suitable base such as potassium carbonate, triethylamine, diisopropylethylamine, pyridine, DMAP or the like at temperature about 25° C. or higher. Preferably, the reaction is carried out in DMA in presence of DMAP at 25° C.

Compound of formula (XXVI) is reacted with alkyl halides in presence of base such as sodium ethoxide, sodium hydride and potassium t-butoxide, to obtain the compound of formula (XXVII), where R^{6a} is selected from substituted- or unsubstituted-alkyl, R² and R^{6b} are same as defined under the compound of formula (I). Preferably, the alkylation reaction is carried out in DMF in presence of sodium hydride.

The compound of formula (XXVII) is further reacted with bromobenzenesulphonamide of formula (V), where R³, R⁴, R⁵ and m are as defined under compound of formula (I), to obtain compound of the formula (Ie), where R^{6a} is selected from substituted- or unsubstituted-alkyl, R², R³, R⁴, R⁵, R^{6b}, and m are as defined under the compound of formula (I). The reaction can be carried out in an organic solvent such as N,N-dimethylformamide, N,N-dimethylacetamide, toluene, or the like in presence of base such as potassium carbonate, potassium phosphate or potassium acetate, and a palladium catalyst. The inventors carried out the said reaction in dimethyl acetamide in presence of potassium acetate and palladium acetate at a temperature of about 150° C.

Scheme 8 below shows route for synthesis of the compound of formula (Ie), where R^{6a} is selected from substituted- or unsubstituted-cycloalkyl, R², R³, R⁴, R⁵, R^{6b}, and m as described under the compound of formula (I), starting from 2,4-dibromothiazole (XX).

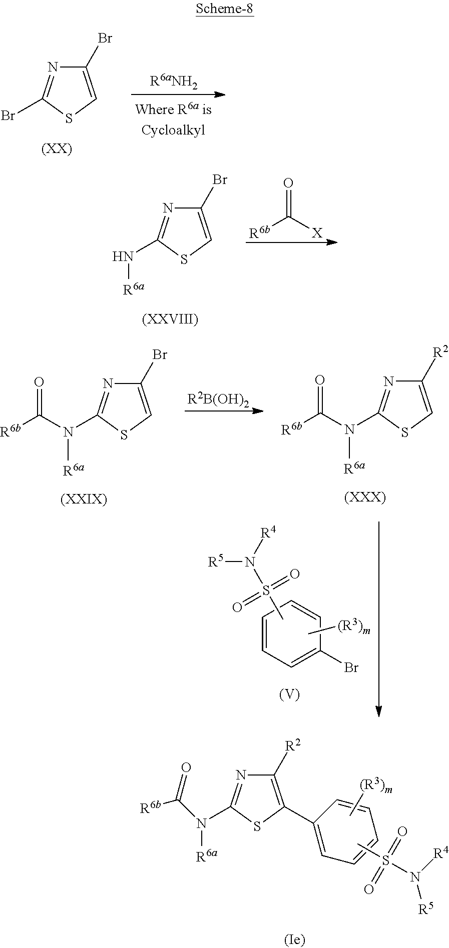

Compound of formula (XX) (2,4-dibromothiazole is commercially available) is reacted with appropriate cycloalkyl amine, to obtain compound of formula (XXVIII), where R^{6a} is selected from substituted- or unsubstituted-cycloalkyl. The reaction can be carried out in an organic solvent such as DMF, THF, a halogenated hydrocarbon such as chloroform and dichloromethane, an aromatic hydrocarbon such as xylene, benzene, toluene, or mixtures thereof or the like (US 2005/153877). Preferably, the reaction is carried out in cycloalkyl amine as solvent.

Compound of formula (XXVIII) is reacted with R^{6b}COX, where R^{6b} is as defined under the compound of formula (I), to obtain compound of formula (XXIX), where R^{6a} is selected from substituted- or unsubstituted-cycloalkyl and R^{6b} is as defined under the compound of formula (I). The reaction can be carried out in the presence of an organic solvent such as DMF, THF, a halogenated hydrocarbon such as chloroform and dichloromethane, an aromatic hydrocarbon such as xylene, benzene, toluene, or mixtures thereof or the like, and a suitable base such as sodium hydride, potassium carbonate, triethylamine, diisopropylethylamine, pyridine, DMAP or the like at temperature about 25° C. or higher. Preferably, the reaction is carried out in THF in presence of sodium hydride at 25° C.

Compound of formula (XXIX) is then subjected to Suzuki coupling with R²B(OH)₂ to obtain the compound of formula (XXX), where R^{6a} is selected from substituted- or unsubstituted-cycloalkyl, R² and R^{6b} are as defined under the compound of formula (I). The reaction with boronic acids and esters can be carried out using conditions well known in the art. Preferably, the Suzuki coupling is carried out in a mixture of ethanol and toluene, in presence of base such as potassium phosphate, potassium carbonate or the like, and tetrakis(triphenylphosphine) palladium(0) at a temperature of about 50° C. or higher.

The compound of formula (XXX) is further reacted with bromobenzenesulphonamide represented by formula (V), where R³, R⁴, R⁵ and m areas defined under compound of formula (I), to obtain compound of the formula (Ie), where R^{6a} is selected from substituted- or unsubstituted-cycloalkyl, R², R³, R⁴, R⁵, R^{6b}, and m are as defined under the compound of formula (I). The reaction can be carried out under the conditions as described earlier in Scheme-7 for the conversion of compound of formula (XXVII) to (le).

The intermediates and the compounds of the present invention may obtained in pure form in a manner known per se, for example, by distilling off the solvent in vacuum and re-crystallizing the residue obtained from a suitable solvent, such as pentane, diethyl ether, isopropyl ether, chloroform, dichloromethane, ethyl acetate, acetone or their combinations or subjecting it to one of the purification methods, such as column chromatography (e.g., flash chromatography) on a suitable support material such as alumina or silica gel using eluent such as dichloromethane, ethyl acetate, hexane, methanol, acetone and their combinations. Preparative LC-MS method is also used for the purification of molecules described herein.

Salts of compound of formula (I) can be obtained by dissolving the compound in a suitable solvent, for example in a chlorinated hydrocarbon, such as methyl chloride or chloroform or a low molecular weight aliphatic alcohol, for example, ethanol or isopropanol, which was then treated with the desired acid or base as described by Stephen M. Berge, et al. “Pharmaceutical Salts, a review article in Journal of Pharmaceutical sciences, 1977, 66 (1), 1-19” and in Handbook of Pharmaceutical Salts, properties, selection, and use by P. Heinrich Stahl and Camille G. Wermuth, Wiley-VCH (2002). Lists of suitable salts can also be found in Remington's Pharmaceutical Sciences, 18th ed., Mack Publishing Company, Easton, Pa., 1990, p. 1445, and Stephen M. Berge et al., Journal of Pharmaceutical Science, 1977, 66 (1), 1-19. For example, they can be a salt of an alkali metal (e.g., sodium or potassium), alkaline earth metal (e.g., calcium), or ammonium of salt.

The compound of the invention or a composition thereof can potentially be administered as a pharmaceutically acceptable acid-addition, base neutralized or addition salt, formed by reaction with inorganic acids, such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base, such as sodium hydroxide, potassium hydroxide. The conversion to a salt is accomplished by treatment of the base compound with at least a stoichiometric amount of an appropriate acid. Typically, the free base is dissolved in an inert organic solvent such as diethyl ether, ethyl acetate, chloroform, ethanol, methanol, and the like, and the acid is added in a similar solvent. The mixture is maintained at a suitable temperature (e.g., between 0° C. and 50° C.). The resulting salt precipitates spontaneously or can be brought out of solution with a less polar solvent.

The stereoisomers of the compound of formula (I) of the present invention may be prepared by stereospecific syntheses or resolution of the achiral compound using an optically active amine, acid or complex forming agent, and separating the diastereomeric salt/complex by fractional crystallization or by column chromatography.

The prodrugs can be prepared in situ during the isolation and purification of the compounds, or by separately reacting the purified compound with a suitable derivatizing agent. For example, hydroxy groups can be converted into esters via treatment with a carboxylic acid in the presence of a catalyst. Examples of cleavable alcohol prodrug moieties include substituted- or unsubstituted-, branched or unbranched lower alkyl ester moieties, e.g., ethyl esters, lower alkenyl esters, di-lower alkylamino lower-alkyl esters, e.g., dimethylaminoethyl ester, acylamino lower alkyl esters, acyloxy lower alkyl esters (e.g., pivaloyloxymethyl ester), aryl esters, e.g., phenyl ester, aryl-lower alkyl esters, e.g., benzyl ester, optionally substituted, e.g., with methyl, halo, or methoxy substituents aryl and aryl-lower alkyl esters, amides, lower-alkyl amides, di-lower alkyl amides, and hydroxy amides.

Modulation of the nicotinic cholinergic receptors, particularly α7 may provide for efficacy in a range of cognitive states, right from pre-attention to attention and subsequently working, reference and recognition memory. Accordingly, this invention may find application in the treatment and prophylaxis of multitude of disease conditions including, either one or combinations of, schizophrenia, schizophreniform disorder, cognitive deficits in schizophrenia, brief psychotic disorder, delusional disorder, schizoaffective disorder, shared psychotic disorder, paranoid personality disorder, schizoid personality disorder, schizotypal personality disorder, attention deficit disorder, attention deficit hyperactivity disorder, depression, maniac depression, major depressive disorder, posttraumatic stress disorder, generalized anxiety disorder, tourette's syndrome, cyclothymic disorder, dysthymic disorder, agoraphobia, panic disorder (with or without agoraphobia), phobias (including social phobia) and bipolar disorders (Morten S. Thomsen, et al., Current Pharmaceutical Design, 2010, 16, 323-343; Peng Zhi-Zhen et al., Zhonghua Yi Xue Yi Chuan Xue Za Zhi, 2008, 25, 154-158; Jared W. Young, et al., European Neuropsychopharmacology, (2007), 17, 145-155; Laura F. Martin, et al., American Journal of Medical Genetics, Part B (Neuropsychiatric Genetics), 2007, 144B, 611-614; Laura F. Martin, et al., Psychopharmacology, (2004), 174, 54-64; Agnes Feher, et al., Dement. Geriatr. Cogn. Disord., 2009, 28, 56-62; Timothy E. Wilens, et al., Biochem. Pharmacol., 2007, 74 (8), 1212-1223; S. L. Verbois, et al., Neuropharmacology, 44 (2003), 224-233; Paul R. Sanberg, et al., Pharmacol. Ther., 1997, 74 (1), 21-25). Cholinergic system, particularly through α7 nAChR seems to have implications in traumatic brain injury-induced psychosis. Chronic nicotine treatment has shown to attenuate same. Thus, this invention may also find application in the treatment of deficits in cholinergic α7 nAChR following traumatic brain injury (M. Bennouna, et al., L'Encephale, 2007, 33, 616-620; S. L. Verbois, et al., Neuropharmacology, 44 (2003), 224-233).

Modulation of nicotinic ACh receptors, particularly the α7 subtype could also help supplement the down-regulated cholinergic receptor expression and transmission as in dementia(s), and also slowing disease progression by reduction of α7-αβ₁₋₄₂ complexation and internalization in AD and Down's syndrome (Agneta Nordberg, et al., Neurotoxicity Research, 2000, 2, 157-165; Simon N. Haydar et al., Bioorganic & Medicinal Chemistry, 17 (2009), 5247-5258; Stephen I. Deutsch et al., Clinical Neuropharmacology, 2003, 26 (5), 277-283). Appropriately, this invention may find application in the treatment and prophylaxis of multitude of disease conditions including, either one or combinations of, dementia(s) due to Alzheimer's disease, dementia with Lewy bodies, Down's syndrome, head trauma, Stroke, hypoperfusion, Parkinson's disease, Huntington's disease, Prion diseases, progressive supranuclear palsy, radiation therapy, brain tumors, normal-pressure hydrocephalus, subdural hematoma, human immunodeficiency virus (HIV) infection, vitamin deficiency, hypothyroidism, drugs, alcohol, lead, mercury, aluminium, heavy metals, syphilis, Lyme disease, viral encephalitis, fungal infection and cryptococcosis (Xilong Zhao et al., Annals New York Academic Science, 2001, 939, 179-186; Elaine Perry et al., European Journal of Pharmacology, 393 (2000), 215-222; C. R. Harrington et al., Dementia, 1994, 5, 215-228; Juan Wang et al., Journal of Neuroscience Research, 88, 807-815 (2010); Kamil Duris et al., Stroke, 2011, 42 (12), 3530-3536). Thus, this invention may also find application in the prophylaxis and preventive measures immediately after early-stage identification of neurodegenerative disease like Alzheimer's disease and Parkinson's disease.

Modulation of nicotinic ACh receptors particularly α4β2, α3β4 and α7 may have implications in the development of therapies for nicotine, cannabis addiction and relapse prevention. Accordingly, this invention may find application in the prophylaxis or therapy of nicotine addiction, cannabis addiction, and relapse prevention of nicotine or cannabis addiction. Additionally, this invention may also provide for an alternative therapy for non-responding addiction patients, patients having intolerable side-effects with de-addiction therapies or those requiring long-term maintenance therapies. (Alexander Kuzmin et al., Psychopharmacology, (2009), 203, 99-108; Robert B. Weiss et al., PLoS Genetics, 2008, 4 (7), e1000125; Marcello Solinas et al., The Journal of Neuroscience, 2007, 27 (21), 5615-5620; Jon O Ebbert et al., Patient Preference and Adherence, 2010, 4, 355-362).

This invention may also find application in the treatment and prophylaxis of multitude of pain conditions including, either one or combinations of, pain arising from, peripheral nervous system (PNS), post-diabetic neuralgia (PDN), post-herpetic neuralgia (PHN), multiple sclerosis, Parkinson's disease, low-back pain, fibromyalgia, post-operative pain, acute pain, chronic pain, mononeuropathy, primary lateral sclerosis, pseudobulbar palsy, progressive muscular palsy, progressive bulbar palsy, postpolio syndrome, diabetes induced polyneuropathy, acute demyelinating polyneuropathy (Guillain-Barre syndrome), acute spinal muscular atrophy (Werdnig-Hoffman disease) and secondary neurodegeneration (Diana L. Donnelly-Roberts et al., Journal of Pharmacology and Experimental Therapeutics, 1998, 285, 777-786; T. J. Rowley et al., British Journal of Anesthesia, 105 (2), 201-207, (2010); A. Bruchfeld et al., Journal of Internal Medicine, 2010, 268, 94-101).

This invention may find application in the treatment and prophylaxis of plethora of inflammation and pain related states involving TNF-α and thus providing symptomatic relief in either any one or combination of, rheumatoid arthritis, bone resorption diseases, atherosclerosis, inflammatory bowel disease, Crohn's disease, inflammation, cancer pain, muscle degeneration, osteoarthritis, osteoporosis, ulcerative colitis, rhinitis, pancreatitis, spondylitis, acute respiratory distress syndrome (ARDS), joint inflammation, anaphylaxis, ischemia reperfusion injury, multiple sclerosis, cerebral malaria, septic shock, tissue rejection of graft, brain trauma, toxic shock syndrome, herpes virus infection (HSV-1 & HSV-2), herpes zoster infection, sepsis, fever, myalgias, asthma, uveititis, contact dermatitis, obesity-related disease and endotoxemia (Ida A. J. Giebelen et al., Shock, 2007, 27 (4), 443-447; Pena Geber et al., Eur. J. Immunol., 2010, 40, 2580-2589).

The invention provides a method of preventing or treating a disease or its symptoms or a disorder mediated partially or completely by nicotinic acetylcholine receptors, said method comprising administering to a subject having or susceptible to said disease or its symptoms or disorder with a therapeutically effective amount of a compound of formula (I), its tautomeric forms, its stereoisomers, or its pharmaceutically acceptable salts.

The disorder, condition, and disease as described above are selected from Alzheimer's disease, mild cognitive impairment, senile dementia, vascular dementia, dementia of Parkinson's disease, attention deficit disorder, attention deficit hyperactivity disorder, dementia associated with Lewy bodies, AIDS dementia complex, Pick's disease, dementia associated with Down's syndrome, Huntington's disease, cognitive deficits associated with traumatic brain injury, cognitive decline associated with stroke, poststroke neuroprotection, cognitive and sensorimotor gating deficits associated with schizophrenia, cognitive deficits associated with bipolar disorder, cognitive impairments associated with depression, acute pain, post-surgical or post-operative pain, chronic pain, inflammation, inflammatory pain, neuropathic pain, smoking cessation, need for new blood vessel growth associated with wound healing, need for new blood vessel growth associated with vascularization of skin grafts, and lack of circulation, arthritis, rheumatoid arthritis, psoriasis, Crohn's disease, ulcerative colitis, pouchitis, inflammatory bowel disease, celiac disease, periodontitis, sarcoidosis, pancreatitis, organ transplant rejection, acute immune disease associated with organ transplantation, chronic immune disease associated with organ transplantation, septic shock, toxic shock syndrome, sepsis syndrome, depression, and rheumatoid spondylitis.

The disease, disorder and condition as described above are particularly selected from the group classified or diagnosed as major or minor neurocognitive disorders, or disorders arising due to neurodegeneration.

The invention further provides a method comprising administering a compound of formula (I) in combination with or as adjunct to medications utilized in the treatment of attention deficit hyperactivity disorders, schizophrenia, cognitive disorders such as Alzheimer's disease, Parkinson's dementia, vascular dementia or dementia associated with Lewy bodies, or traumatic brain injury.

The method as described above further comprising administering a compound of formula (I) in combination with or as an adjunct to acetylcholinesterase inhibitors, disease modifying drugs or biologics for neurodegenerative disorders, dopaminergic drugs, antidepressants, or a typical or an atypical antipsychotic.

The invention also provides use of a compound of formula (I), its tautomeric forms, its stereoisomers, and its pharmaceutically acceptable salts in preparation of a medicament for preventing or treating a disease or its symptoms or a disorder mediated partially or completely by nicotinic acetylcholine receptors.

The use as described above, wherein, the disease or disorder or condition is selected from the group classified or diagnosed as major or minor neurocognitive disorders, or disorders arising due to neurodegeneration.

The use as described above is in combination with or as adjunct to medications utilized in the treatment of attention deficit hyperactivity disorders, schizophrenia, cognitive disorders, Alzheimer's disease, Parkinson's dementia, vascular dementia or dementia associated with Lewy bodies, and traumatic brain injury.

The use as described above is in combination with or as an adjunct to acetylcholinesterase inhibitors, disease modifying drugs or biologics for neurodegenerative disorders, dopaminergic drugs, antidepressants, or a typical or atypical antipsychotic.

Following are the abbreviations used and meaning thereof in the specification:

### ACh: Acetylcholine.

AD: Alzheimer's disease.
AIDS: Acquired immunodeficiency syndrome.
DCM: dichloromethane.

### DMF: N,N-dimethylformamide.

### FLIPR: Fluorometric Imaging Plate Reader.

HBSS: Hank's balanced salt solution.
HEPES: 4-(2-hydroxyethyl)piperazine-1-ethanesulfonic acid.
HOBT: hydroxybenzotriazole hydrate.
HPLC: High Performance liquid chromatography.
PAM: positive allosteric modulation.

### THF: Tetrahydrofuran.

TLC: Thin layer chromatography.
TNF-α: tumor necrosis factor alpha.
α7 nAChR: nicotinic acetylcholine receptor α7 subunit.

Following examples are provided to further illustrate the present invention and therefore should not be construed in any way to limit the scope of the present invention. All ¹H NMR spectra were determined in the solvents indicated and chemical shifts are reported in δ units downfield from the internal standard tetramethylsilane (TMS) and interproton coupling constants are reported in Hertz (Hz).

### Example 1

### 4-(4-(4-chlorophenyl)-2-(cyclopentanecarbonyl)thiazol-5-yl)benzenesulfonamide (Compound 1)

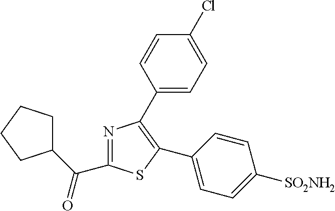 [SMILES: NS(=O)(=O)c1ccc(-c2sc(C(=O)C3CCCC3)nc2-c2ccc(Cl)cc2)cc1]

### Step 1: 4-(4-chlorophenyl)-N-methoxy-N-methylthiazole-2-carboxamide (1a)

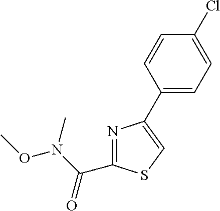 [SMILES: CON(C)C(=O)c1nc(-c2ccc(Cl)cc2)cs1]

HOBT (4.79 g, 31.3 mmol) and EDC (6.0 g, 31.3 mmol) were added to a solution of 4-(4-chlorophenyl)thiazole-2-carboxylic acid (Prepared according to procedure US 2007/32531, 5.0 g, 20.86 mmol) in DMF (50 ml) at 0° C. N,O-dimethylhydroxylamine hydrochloride (2.44 g, 25.03 mmol) and triethylamine (10.55 g, 14.54 ml, 104.0 mmol) were added and the reaction mixture was stirred at room temperature for 18 hr. The progress of the reaction was monitored by TLC. The reaction mixture concentrated under reduced pressure to remove solvent, residue was diluted with ethyl acetate (150 ml). Organic layer was washed with saturated sodium bicarbonate solution (50 ml), brine (50 ml). Organic layer was dried over anhydrous Na₂SO₄. The solvent was evaporated under reduced pressure to obtain a crude product; the crude product was stirred in hexanes (75 ml) for 15 min and filtered to obtain the title compound (4.50 g, 76.0%). MS: m/z 283 (M+1).

¹H NMR (CDCl₃, 400 MHz): δ 7.87 (d, J=8.4 Hz, 2H), 7.73 (s, 1H), 7.41 (d, J=8.4 Hz, 2H), 3.94 (s, 3H), 3.63 (s, 3H).

### Step 2: (4-(4-chlorophenyl)thiazol-2-yl)(cyclopentyl)methanone (1b)

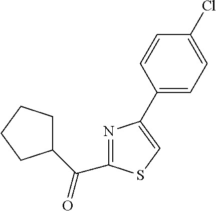 [SMILES: O=C(c1nc(-c2ccc(Cl)cc2)cs1)C1CCCC1]

Cyclopentylmagnesium bromide (0.76 g, 4.42 ml (2 M solution in Diethyl ether) 21.10 mmol) was added to a solution of 4-(4-chlorophenyl)-N-methoxy-N-methylthiazole-2-carboxamide (Compound 1a, 0.50 g, 1.77 mmol) in THF (15 ml) at 0° C. The reaction mixture was stirred at room temperature for 1 hr. The progress of the reaction was monitored by TLC. The reaction mixture was quenched with saturated solution of ammonium chloride (2 ml) and extracted with ethyl acetate (2×30 ml). Organic layer washed with brine (25 ml). The organic layer separated was dried over anhydrous sodium sulphate and concentrated under reduced pressure to obtain a crude product. The crude product was further purified by flash column chromatography using dichloromethane as an eluent to obtain the title compound (0.245 g, 47.5%). MS: m/z 292 (M+1).

¹H NMR (CDCl₃, 400 MHz): δ 7.90 (d, J=8.8 Hz, 2H), 7.79 (s, 1H), 7.44 (d, J=8.8 Hz, 2H), 4.09-4.13 (m, 1H), 2.09-2.50 (m, 2H), 1.95-1.98 (m, 2H), 1.70-1.80 (m, 2H), 1.51-1.59 (m, 2H).

### Step 3: 4-(4-(4-chlorophenyl)-2-(cyclopentanecarbonyl)thiazol-5-yl)benzenesulfonamide (Compound 1)

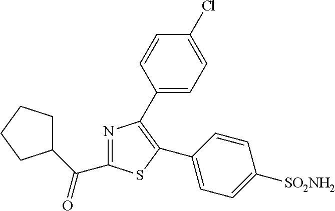 [SMILES: NS(=O)(=O)c1ccc(-c2sc(C(=O)C3CCCC3)nc2-c2ccc(Cl)cc2)cc1]

4-bromobenzenesulfonamide (0.23 g, 0.98 mmol) and potassium acetate (0.20 g, 2.05 mmol) were added to a solution of (4-(4-chlorophenyl)thiazol-2-yl)(cyclopentyl)methanone (Compound 1b, 0.24 g, 0.82 mmol) in a DMA (5 ml) in a tube at 25° C. Nitrogen gas was bubbled through the reaction mixture for 15 minutes. Palladium acetate (0.009 g, 0.04 mmol) was then added to the reaction mixture under nitrogen atmosphere and the tube was sealed. The reaction mixture was heated at 150° C. for 16 hr under stirring. The progress of the reaction was monitored by TLC. The reaction mixture was then cooled to 25° C. and concentrated under reduced pressure to remove solvent; residue was diluted with ethyl acetate (10 ml) and filtered through celite. The celite cake was washed with ethyl acetate (10 ml). The combined filtrate was concentrated under reduced pressure to obtain a crude product, which was then purified by preparative HPLC to obtain the title compound (0.08 g, 21.7%). MS: m/z 447 (M+1).

¹H NMR (DMSO-d₆, 400 MHz): δ 7.85 (d, J=8.4 Hz, 2H), 7.62 (d, J=8.4 Hz, 2H), 7.45-7.55 (m, 6H), 3.96-4.03 (m, 1H), 1.98-2.03 (m, 2H), 1.81-1.86 (m, 2H), 1.61-1.69 (m, 4H).

The following compound was prepared according to the procedure described above for compound 1, with appropriate changes to the reactants.

4-(4-(4-chlorophenyl)-2-(cyclopentanecarbonyl)thiazol-5-yl)-3-fluorobenzenesulfonamide (Compound 2). MS: m/z 465 (M+1).

¹H NMR (DMSO-d₆, 400 MHz): δ 7.71-7.78 (m, 3H), 7.65 (bs-exchanges with D₂O, 2H), 7.46-7.49 (m, 4H), 3.99-4.03 (m, 1H), 1.99-2.04 (m, 2H), 1.83-1.86 (m, 2H), 1.67-2.70 (m, 4H).

### Example 2

### 4-(4-(4-chlorophenyl)-2-picolinoylthiazol-5-yl)benzenesulfonamide (Compound 3)

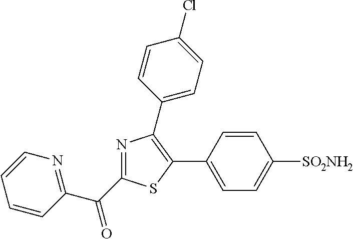 [SMILES: NS(=O)(=O)c1ccc(-c2sc(C(=O)c3ccccn3)nc2-c2ccc(Cl)cc2)cc1]

### Step 1: (4-(4-chlorophenyl)thiazol-2-yl)(pyridin-2-yl)methanone (3a)

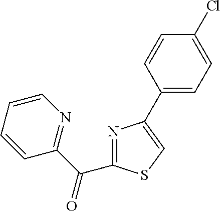 [SMILES: O=C(c1ccccn1)c1nc(-c2ccc(Cl)cc2)cs1]

Isopropyl magnesium chloride (2.17 g, 10.5 ml (2M solution in THF) 21.10 mmol) was added to a solution of 2-bromopyridine (2.0 g, 12.66 mmol) in THF (50 ml) at 0° C. The reaction mixture was further stirred at room temperature for 4 hours. Solution of 4-(4-chlorophenyl)-N-methoxy-N-methylthiazole-2-carboxamide (Compound 1a, 1.19 g, 4.22 mmol) in THF (25 ml) was added to a reaction mixture slowly at 0° C. The reaction mixture was stirred at room temperature for 1 hr. The progress of the reaction was monitored by TLC. The reaction mixture was quenched with saturated solution of ammonium chloride (5 ml) and extracted with ethyl acetate (2×50 ml). Organic layer washed with brine (25 ml). The organic layer separated was dried over anhydrous sodium sulphate and concentrated under reduced pressure to obtain a crude product. The crude product was further purified by column chromatography using 15% ethyl acetate in hexanes as an eluent to obtain the title compound (1.0 g, 79.0%). MS: m/z 301 (M+1).

¹H NMR (CDCl₃, 400 MHz): δ 8.42-8.44 (m, 1H), 7.97-8.00 (m, 4H), 7.93 (s, 1H), 7.60 (ddd, J=8.0 Hz, 7.6 Hz, 1.6 Hz, 1H), 7.42-7.46 (m, 2H).

The compound given below was prepared by procedure similar to the one described above for compound ‘3a’ with appropriate variations of reactants, reaction conditions and quantities of reagents.

4a. (4-(4-chlorophenyl)thiazol-2-yl)(pyridin-3-yl)methanone. MS: m/z 301 (M+1).

### Step 2: 4-(4-(4-chlorophenyl)-2-picolinoylthiazol-5-yl)benzenesulfonamide (Compound 3)

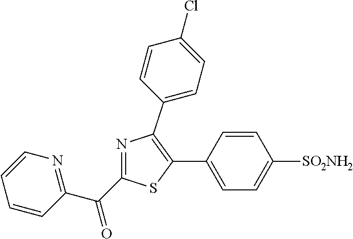 [SMILES: NS(=O)(=O)c1ccc(-c2sc(C(=O)c3ccccn3)nc2-c2ccc(Cl)cc2)cc1]

4-bromobenzenesulfonamide (0.43 g, 1.83 mmol) and potassium acetate (0.326 g, 3.32 mmol) were added to a solution of (4-(4-chlorophenyl)thiazol-2-yl)(pyridin-2-yl)methanone (Compound 3a, 0.5 g, 1.662 mmol) in a DMA (5 ml) in a tube at 25° C. Nitrogen gas was bubbled through the reaction mixture for 15 minutes. Palladium acetate (0.037 g, 0.16 mmol) was then added to the reaction mixture under nitrogen atmosphere and the tube was sealed. The reaction mixture was heated at 150° C. for 18 hrs under stirring. The progress of the reaction was monitored by TLC. The reaction mixture was then concentrated under reduced pressure to remove solvent, residue was diluted with ethyl acetate (20 ml) and filtered through celite. The celite cake was washed with ethyl acetate (20 ml). The combined filtrate was concentrated under reduced pressure to obtain a crude product, which was then purified by column chromatography using 35% ethyl acetate in hexanes as an eluent to obtain the title compound (0.025 g, 3.30%). MS: m/z 456 (M+1).

¹H NMR (DMSO-d₆, 400 MHz): δ 8.86 (m, 1H), 8.29 (d, J=7.6 Hz, 1H), 8.14-8.18 (m, 1H), 7.88 (d, J=8.4 Hz, 2H), 7.78-7.82 (m, 1H), 7.67 (d, J=8.4 Hz, 2H), 7.48-7.53 (m, 6H).

The following compound was prepared according to the procedure described above for compound 3, with appropriate changes to the reactants.

4-(4-(4-chlorophenyl)-2-nicotinoylthiazol-5-yl)benzenesulfonamide (Compound 4). [MS: m/z 456 (M+1)].

¹H NMR (DMSO-d₆, 400 MHz): δ 9.54 (d, J=1.6 Hz, 1H), 8.88-8.89 (m, 1H), 8.72 (d, J=8.0 Hz, 1H), 7.88 (d, J=8.0 Hz, 2H), 7.66-7.69 (m, 3H), 7.49-7.55 (m, 6H).

### Example 3

### 4-(4-(4-chlorophenyl)-2-(1-cyclopropyl-2-methyl-1-oxopropan-2-yl)thiazol-5-yl)-3-fluorobenzenesulfonamide (Compound 5)

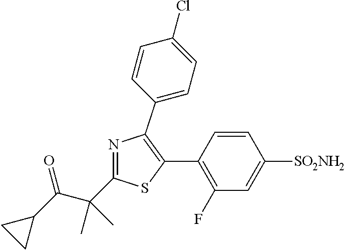 [SMILES: CC(C)(C(=O)C1CC1)c1nc(-c2ccc(Cl)cc2)c(-c2ccc(S(N)(=O)=O)cc2F)s1]

### Step 1: 2-(4-(4-chlorophenyl)thiazol-2-yl)-N-methoxy-N-methylacetamide (5a)

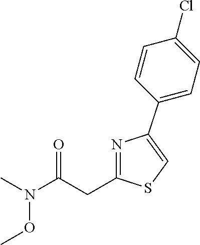 [SMILES: CON(C)C(=O)Cc1nc(-c2ccc(Cl)cc2)cs1]

HOBT (4.53 g, 29.6 mmol) and EDC (5.67 g, 29.6 mmol) were added to a solution of 2-(4-(4-chlorophenyl)thiazol-2-yl)acetic acid (Prepared according to procedure reported in US 2007/32531, 5.0 g, 19.71 mmol) in DMF (50 ml) at 0° C. N,O-dimethylhydroxylamine hydrochloride (2.11 g, 21.68 mmol) and triethylamine (9.97 g, 13.73 ml, 99.0 mmol) were added and the reaction mixture was stirred at room temperature for 18 hr. The progress of the reaction was monitored by TLC. The reaction mixture was diluted with cold water (100 ml). Solid precipitated out was filtered and dried to obtain the title compound (5.85 g, 85%). MS: m/z 297 (M+1).

¹H NMR (CDCl₃, 400 MHz): δ 7.83 (d, J=8.4 Hz, 2H), 7.34 (s, 1H), 7.39 (d, J=8.4 Hz, 2H), 4.30 (s, 2H), 3.79 (s, 3H) 3.28 (s, 3H).

### Step 2: 2-(4-(4-chlorophenyl)thiazol-2-yl)-N-methoxy-N,2-dimethylpropanamide (5b)

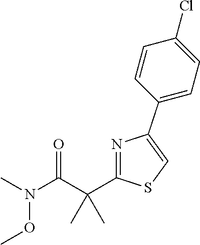 [SMILES: CON(C)C(=O)C(C)(C)c1nc(-c2ccc(Cl)cc2)cs1]

Sodium methoxide (1.00 g, 14.83 mmol) was added to a solution of 2-(4-(4-chlorophenyl)thiazol-2-yl)-N-methoxy-N-methylacetamide (Compound 5a, 2.00 g, 6.74 mmol) in DMF (10 ml) at 0° C. Stirred for 15 minutes and then added methyl iodide (2.00 g, 0.88 ml, 14.15 mmol). The reaction mixture was stirred at room temperature for 4 hr. The progress of the reaction was monitored by TLC. The reaction mixture was diluted with water (100 ml) and extracted with ethyl acetate (2×100 ml). Organic layer was washed with brine (100 ml). The organic layer separated was dried over anhydrous sodium sulphate and concentrated under reduced pressure to obtain a crude product. The crude product was further purified by column chromatography using 10% ethyl acetate in hexanes as an eluent to obtain the title compound (1.2 g, 54.8%). MS: m/z 325 (M+1).

¹H NMR (CDCl₃, 400 MHz): δ 7.87 (d, J=8.4 Hz, 2H), 7.38 (d, J=8.4 Hz, 2H), 7.37 (s, 1H), 3.20 (s, 3H), 2.98 (s, 3H), 1.71 (s, 6H).

### Step 3: 2-(4-(4-chlorophenyl)thiazol-2-yl)-1-cyclopropyl-2-methylpropan-1-one (5c)

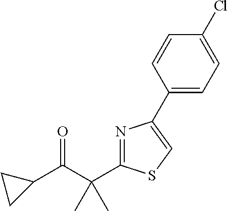 [SMILES: CC(C)(C(=O)C1CC1)c1nc(-c2ccc(Cl)cc2)cs1]

Cyclopropyl magnesium bromide (3.58 g, 49.30 ml (0.5 M solution in THF), 24.63 mmol) was added to a solution of 2-(4-(4-chlorophenyl)thiazol-2-yl)-N-methoxy-N,2-dimethylpropanamide (Compound 5b, 0.80 g, 2.46 mmol) in THF (10 ml) at 0° C. The reaction mixture was stirred at room temperature for 2 hr. The progress of the reaction was monitored by TLC. The reaction mixture was quenched with saturated solution of ammonium chloride (2 ml) and extracted with ethyl acetate (2×50 ml). Organic layer was washed with brine (50 ml). The organic layer separated was dried over anhydrous sodium sulphate and concentrated under reduced pressure to obtain a crude product. The crude product was further purified by column chromatography using 5% ethyl acetate in hexanes as an eluent to obtain the title compound (0.5 g, 66.4%). MS: m/z 306 (M+1).

¹H NMR (CDCl₃, 400 MHz): δ 7.88 (d, J=7.6, 8.4 Hz, 2H), 7.45 (s, 1H), 7.40 (d, J=8.4 Hz, 2H), 2.04-2.09 (m, 1H), 1.73 (s, 6H), 1.00-1.04 (m, 2H), 0.79-0.84 (m, 2H).

The compound given below was prepared by procedure similar to the one described above for compound ‘5c’ with appropriate variations of reactants, reaction conditions and quantities of reagents.

7c. (1-(4-(4-chlorophenyl)thiazol-2-yl)cyclopropyl)(cyclopropyl)methanone. MS: m/z 302 (M+1).

### Step 4: 4-(4-(4-chlorophenyl)-2-(1-cyclopropyl-2-methyl-1-oxopropan-2-yl)thiazol-5-yl)-3-fluorobenzenesulfonamide (Compound 5)

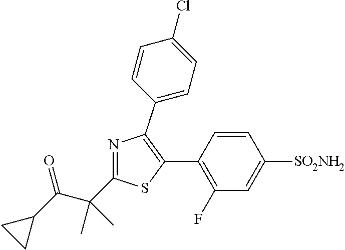 [SMILES: CC(C)(C(=O)C1CC1)c1nc(-c2ccc(Cl)cc2)c(-c2ccc(S(N)(=O)=O)cc2F)s1]

4-bromo-3-fluorobenzenesulfonamide (0.46 g, 1.79 mmol) and potassium acetate (0.32 g, 3.27 mmol) were added to a solution of 2-(4-(4-chlorophenyl)thiazol-2-yl)-1-cyclopropyl-2-methylpropan-1-one (Compound 5c, 0.5 g, 1.63 mmol) in a DMA (8 ml) in a tube at 25° C. Nitrogen gas was bubbled through the reaction mixture for 15 minutes. Palladium acetate (0.037 g, 0.16 mmol) was then added to the reaction mixture under nitrogen atmosphere and the tube was sealed. The reaction mixture was heated at 130° C. for 1 hr. under stirring. The progress of the reaction was monitored by TLC. The reaction mixture was then concentrated under reduced pressure to remove solvent, residue was diluted with ethyl acetate (20 ml) and filtered through celite. The celite cake was washed with ethyl acetate (10 ml). The combined filtrate was concentrated under reduced pressure to obtain a crude product, which was then purified by column chromatography using 20% ethyl acetate in hexanes as an eluent to obtain the title compound (0.31 g, 40.1%). MS: m/z 479 (M+1)].

¹H NMR (CDCl₃, 400 MHz): δ 7.68-7.72 (m, 2H), 7.42-7.48 (m, 3H), 7.28-7.30 (m, 2H), 5.05 (bs exchanges with D₂O, 2H), 2.15-2.21 (m, 1H), 1.76 (s, 6H) 1.05-1.09 (m, 2H), 0.88-0.92 (m, 2H).

The following compounds were prepared according to the procedure described above for compound 5, with appropriate changes to the reactants.

4-(4-(4-chlorophenyl)-2-(1-cyclopropyl-2-methyl-1-oxopropan-2-yl)thiazol-5-yl)benzenesulfonamide (Compound 6). [MS: m/z 461 (M+1)].

¹H NMR (DMSO-d₆, 400 MHz): δ 7.82 (d, J=8.0 Hz, 2H), 7.55 (d, J=8.0 Hz, 2H), 7.47 (bs-exchanges with D₂O, 2H), 7.43-7.45 (m, 4H), 2.20-2.24 (m, 1H), 1.67 (s, 6H), 0.83-0.93 (m, 4H).

4-(4-(4-chlorophenyl)-2-(1-(cyclopropanecarbonyl)cyclopropyl)thiazol-5-yl)-3-fluorobenzenesulfonamide (Compound 7). [MS: m/z 477 (M+1)].

¹H NMR (CDCl₃, 400 MHz): δ 7.68 (d, J=8.0 Hz, 2H), 7.47 (t, J=8.0 Hz, 1H), 7.39 (d, J=8.0 Hz, 2H), 7.26-7.28 (m, 2H), 4.90 (bs-exchanges with D₂O, 2H), 2.02-2.15 (m, 2H), 1.74-1.79 (m, 1H), 1.22-1.27 (m, 2H), 1.16-1.19 (m, 2H), 0.91-0.98 (m, 2H).

### Example 4

### 4-(4-(4-chlorophenyl)-2-((2-oxocyclopentyl)methyl)thiazol-5-yl)benzenesulfonamide (Compound 8)

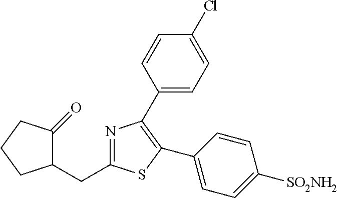 [SMILES: NS(=O)(=O)c1ccc(-c2sc(CC3CCCC3=O)nc2-c2ccc(Cl)cc2)cc1]

### Step 1: methyl 1-((4-(4-chlorophenyl)thiazol-2-yl)methyl)-2-oxocyclopentanecarboxylate (8a)

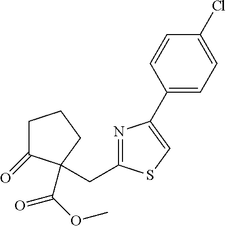 [SMILES: COC(=O)C1(Cc2nc(-c3ccc(Cl)cc3)cs2)CCCC1=O]

Potassium carbonate (1.39 g, 10.12 mmol) was added to a solution of methyl 2-oxocyclopentanecarboxylate (0.36 g, 0.31 ml, 2.53 mmol) in anhydrous acetone (20 ml). Reaction mixture was stirred at 25° C. for 30 min. A solution of 2-(bromomethyl)-4-(4-chlorophenyl)thiazole (prepared according to literature WO 2007/107758, 0.73 g, 2.53 mmol) in anhydrous acetone (10 ml) was added slowly and reaction mixture was heated to reflux temperature for 1 hr. The progress of the reaction was monitored by TLC. The reaction mixture was filtered and washed with acetone (10 ml), combined filtrate was concentrated under reduced pressure to remove solvent. Residue was diluted with ethyl acetate (50 ml), washed with water (25 ml), brine (25 ml). Organic layer was dried over anhydrous sodium sulphate. The solvent was evaporated under reduced pressure to obtain the title compound (0.8 g, 90%). MS: m/z 350 (M+1).

¹H NMR (DMSO-d₆, 400 MHz): δ 8.06 (s, 1H), 7.91 (d, J=8.4 Hz, 2H), 7.50 (d, J=8.4 Hz, 2H), 3.66 (s, 3H), 3.63 (s, 2H), 2.38-2.46 (m, 2H), 2.02-2.31 (m, 2H), 1.83-2.01 (m, 2H).

### Step 2: 2-((4-(4-chlorophenyl)thiazol-2-yl)methyl)cyclopentanone (8b)

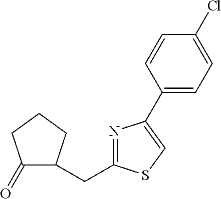 [SMILES: O=C1CCCC1Cc1nc(-c2ccc(Cl)cc2)cs1]

Acetic acid (5 ml) and concentrated HCl (2 ml) were added to a methyl 1-((4-(4-chlorophenyl)thiazol-2-yl)methyl)-2-oxocyclopentanecarboxylate (Compound 8a, 30.87 g, 2.50 mmol) at room temperature in a tube and tube was sealed. Reaction mixture was refluxed at 105° C. for 16 hr. The progress of the reaction was monitored by TLC. The reaction mixture was concentrated under reduced pressure, neutralized with saturated solution of sodium carbonate (5 ml) and extracted with ethyl acetate (2×30 ml) and washed with brine (25 ml). The organic layer separated was dried over anhydrous sodium sulphate and concentrated under reduced pressure to obtain a crude product. The crude product was further purified by flash column chromatography using 40% ethyl acetate in hexanes as an eluent to obtain the title compound (0.358 g, 49.1%). MS: m/z 292 (M+1).

¹H NMR (CDCl₃, 400 MHz): δ 7.82 (d, J=8.4 Hz, 2H), 7.39 (d, J=8.4 Hz, 2H), 7.34 (s, 1H), 3.53 (dd, J=15.6 Hz, 6.8 Hz, 1H), 3.08 (dd, J=15.6 Hz, 6.8 Hz, 1H), 2.61-2.72 (m, 1H), 2.31-2.50 (m, 2H), 2.18-2.58 (m, 1H), 2.06-2.18 (m, 1H), 1.78-1.86 (m, 1H), 1.70-1.75 (m, 1H).

### Step 3: 4-(4-(4-chlorophenyl)-2-((2-oxocyclopentyl)methyl)thiazol-5-yl)benzenesulfonamide (Compound 8)

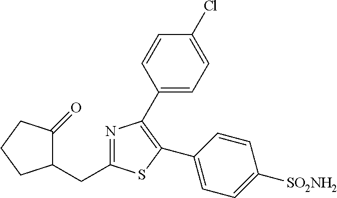 [SMILES: NS(=O)(=O)c1ccc(-c2sc(CC3CCCC3=O)nc2-c2ccc(Cl)cc2)cc1]

4-bromobenzenesulfonamide (0.14 g, 0.60 mmol) and potassium acetate (0.14 g, 1.49 mmol) were added to a solution of 2-((4-(4-chlorophenyl)thiazol-2-yl)methyl)cyclopentanone (Compound 8b, 0.17 g, 0.60 mmol) in a DMA (5 ml) in a tube at 25° C. Nitrogen gas was bubbled through the reaction mixture for 15 minutes. Palladium acetate (0.007 g, 0.03 mmol) was then added to the reaction mixture under nitrogen atmosphere and the tube was sealed. The reaction mixture was heated at 150° C. for 16 hr. under stirring. The progress of the reaction was monitored by TLC. The reaction mixture was then concentrated under reduced pressure to remove solvent, residue was diluted with ethyl acetate (20 ml) and filtered through celite. The celite cake was washed with ethyl acetate (10 ml). The combined filtrate was concentrated under reduced pressure to obtain a crude product, which was then purified by column chromatography using 60% ethyl acetate in hexanes as an eluent to obtain the title compound (0.074 g, 27.6%). MS: m/z 447 (M+1).

¹H NMR (DMSO-d₆, 400 MHz): δ 7.81 (d, J=6.8 Hz, 2H), 7.51 (d, J=6.8 Hz, 2H), 7.45 (bs-exchanges with D₂O, 2H), 7.42 (s, 4H), 3.34-3.39 (m, 1H), 3.02-3.08 (m, 1H), 2.62-2.71 (m, 1H), 2.11-2.46 (m, 3H), 1.88-1.99 (m, 1H), 1.63-1.84 (m, 2H).

The following compound was prepared according to the procedure described above for compound 8, with appropriate changes to the reactants.

4-(4-(4-chlorophenyl)-2-((2-oxocyclopentyl)methyl)thiazol-5-yl)-3-fluorobenzenesulfonamide (Compound 9). [MS: m/z 465 (M+1)].

¹H NMR (DMSO-d₆, 400 MHz): δ 7.64-7.71 (m, 3H), 7.60 (bs-exchanges with D₂O, 2H), 7.40 (s, 4H), 3.37-3.42 (m, 1H), 3.05-3.11 (m, 1H), 2.62-2.72 (m, 1H), 2.06-2.32 (m, 3H), 1.90-2.02 (m, 1H), 1.63-1.72 (m, 2H).

### Example 5

### 4-(2-(2-(3-azabicyclo[3.1.0]hexan-3-yl)-2-oxoethyl)-4-(4-chlorophenyl)thiazol-5-yl)benzenesulfonamide (Compound 10)

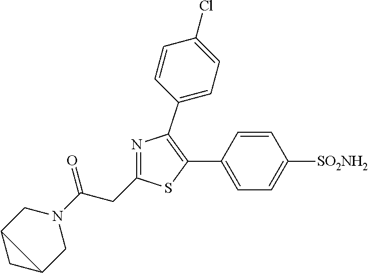 [SMILES: NS(=O)(=O)c1ccc(-c2sc(CC(=O)N3CC4CC4C3)nc2-c2ccc(Cl)cc2)cc1]

### Step 1: 1-(3-azabicyclo[3.1.0]hexan-3-yl)-2-(4-(4-chlorophenyl)thiazol-2-yl)ethanone (10a)

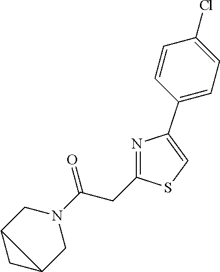 [SMILES: O=C(Cc1nc(-c2ccc(Cl)cc2)cs1)N1CC2CC2C1]

HOBT (0.45 g, 2.96 mmol) was added to a solution of 2-(4-(4-chlorophenyl)thiazol-2-yl)acetic acid (Prepared according to the procedure reported in the literature US 2007/32531, 0.50 g, 1.97 mmol) in DMF (5 ml) under stirring at room temperature. 3-azabicyclo[3.1.0]hexane hydrochloride (0.23 g, 1.97 mmol) was then added to the reaction mixture. The reaction mixture was cooled to 0° C. and EDC (0.56 g, 2.96 mmol) and triethylamine (0.99 g, 1.37 ml, and 9.85 mmol) were added to it. The reaction mixture was stirred at room temperature for 15 hours. The progress of the reaction was monitored by TLC. The reaction mixture was concentrated under reduced pressure. The residue obtained after concentration was mixed with ethyl acetate (100 ml) and the resultant mixture was washed with saturated sodium bicarbonate solution (30 ml) followed by washing with brine (30 ml). The organic layer separated was dried over anhydrous sodium sulphate and concentrated under reduced pressure to obtain a crude product. The crude product was further purified by column chromatography using 20% ethyl acetate in hexanes as an eluent to obtain the title compound (0.45 g, 71.6%). MS: m/z 319 (M+1).

¹H NMR (CDCl₃, 400 MHz): δ 7.82 (d, J=8.8 Hz, 2H), 7.44 (s, 1H), 7.38 (d, J=8.8 Hz, 2H), 4.08 (s, 2H), 3.86 (d, J=11.6 Hz, 1H), 3.78 (d, J=9.6 Hz, 1H), 3.71 (dd, J=9.6, 4.0 Hz, 1H), 3.46 (dd, J=11.6, 4.0 Hz, 1H), 1.57-1.66 (m, 2H), 0.75-0.79 (m, 1H), 0.20-0.23 (m, 1H).

The compounds given below were prepared by procedure similar to the one described above for compound ‘10a’ with appropriate variations of reactants, reaction conditions and quantities of reagents.

11a. (cis)-2-(4-(4-chlorophenyl)thiazol-2-yl)-1-(2,6-dimethylmorpholino)ethanone. MS: m/z 351 (M+1).

12a. 2-(4-(4-chlorophenyl)thiazol-2-yl)-1-(pyrrolidin-1-yl)ethanone. MS: m/z 351 (M+1).

### Step 2: 4-(2-(2-(3-azabicyclo[3.1.0]hexan-3-yl)-2-oxoethyl)-4-(4-chlorophenyl)thiazol-5-yl)benzenesulfonamide (Compound 10)

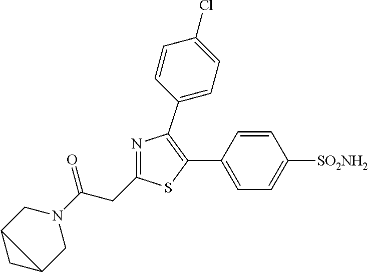 [SMILES: NS(=O)(=O)c1ccc(-c2sc(CC(=O)N3CC4CC4C3)nc2-c2ccc(Cl)cc2)cc1]

4-bromobenzenesulfonamide (0.37 g, 1.55 mmol) and potassium acetate (0.27 g, 2.82 mmol) were added to a solution of 1-(3-azabicyclo[3.1.0]hexan-3-yl)-2-(4-(4-chlorophenyl)thiazol-2-yl)ethanone (Compound 10a, 0.45 g, 1.41 mmol) in a DMA (5 ml) in a tube at 25° C. Nitrogen gas was bubbled through the reaction mixture for 15 minutes. Palladium acetate (0.03 g, 0.14 mmol) was then added to the reaction mixture under nitrogen atmosphere and the tube was sealed. The reaction mixture was heated at 150° C. for 20 hrs under stirring. The progress of the reaction was monitored by TLC. The reaction mixture was then cooled to 25° C. and filtered through celite. The celite cake was washed with ethyl acetate (30 ml). The combined filtrate was concentrated under reduced pressure to obtain a crude product, which was then purified by column chromatography using 40% ethyl acetate in hexanes as an eluent to obtain the title compound (0.045 g, 6.73%). MS: m/z 474 (M+1).

¹H NMR (DMSO-d₆, 400 MHz): δ 7.82 (d, J=8.8 Hz, 2H), 7.52 (d, J=8.8 Hz, 2H), 7.45 (bs-exchanges with D₂O, 2H), 7.42 (s, 4H), 4.19 (q, J=18.0 Hz, 2H), 3.72 (d, J=10.4 Hz, 1H), 3.62-3.72 (m, 2H), 3.42-3.48 (m, 1H), 1.55-1.66 (m, 2H), 0.71-0.75 (m, 1H), 0.13-0.16 (m, 1H).

The following compounds were prepared according to the procedure described above for compound 10, with appropriate changes to the reactants.

(cis)-4-(4-(4-chlorophenyl)-2-(2-(2,6-dimethylmorpholino)-2-oxoethyl)thiazol-5-yl)benzenesulfonamide (Compound 11). MS: m/z 506 (M+1).

¹H NMR (DMSO-d₆, 400 MHz): δ 7.82 (d, J=8.4 Hz, 2H), 7.53 (d, J=8.4 Hz, 2H), 7.45 (bs-exchanges with D₂O, 2H), 7.40-7.42 (s, 4H), 4.37-4.42 (m, 1H), 4.22-4.32 (m, 2H), 3.96-4.01 (m, 1H), 3.50-3.62 (m, 2H), 3.40-3.48 (m, 2H), 1.09-1.14 (m, 6H).

4-(4-(4-chlorophenyl)-2-(2-oxo-2-(pyrrolidin-1-yl)ethyl)thiazol-5-yl)benzenesulfonamide (Compound 12). MS: m/z 462 (M+1).

¹H NMR (DMSO-d₆, 400 MHz): δ 7.82 (d, J=8.4 Hz, 2H), 7.53 (d, J=8.4 Hz, 2H), 7.44 (bs-exchanges with D₂O, 2H), 7.40-7.42 (s, 4H), 4.21 (s, 2H), 3.56 (t, J=6.8 Hz, 2H), 3.35 (t, J=6.8 Hz, 2H), 1.90-1.98 (m, 2H), 1.75-1.83 (m, 2H).

### Example 6

### 4-(2-(1-(3-azabicyclo[3.1.0]hexan-3-yl)-2-methyl-1-oxopropan-2-yl)-4-(4-chlorophenyl)thiazol-5-yl)benzenesulfonamide (Compound 13)

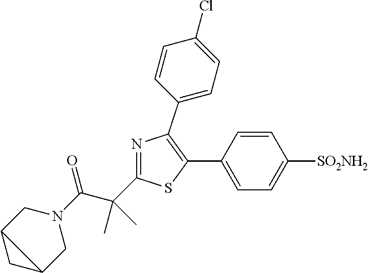 [SMILES: CC(C)(C(=O)N1CC2CC2C1)c1nc(-c2ccc(Cl)cc2)c(-c2ccc(S(N)(=O)=O)cc2)s1]

### Step 1: 1-(3-azabicyclo[3.1.0]hexan-3-yl)-2-(4-(4-chlorophenyl)thiazol-2-yl)-2-methylpropan-1-one (13a)

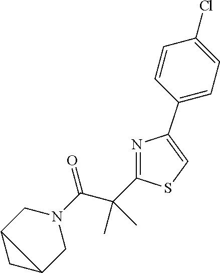 [SMILES: CC(C)(C(=O)N1CC2CC2C1)c1nc(-c2ccc(Cl)cc2)cs1]

Sodium ethoxide (0.23 g, 3.45 mmol) was added to a solution of 1-(3-azabicyclo[3.1.0]hexan-3-yl)-2-(4-(4-chlorophenyl)thiazol-2-yl) ethanone (Compound 10a, 0.5 g, 1.57 mmol) in DMF (10 ml) under nitrogen atmosphere with stirring at 0° C. To the reaction mixture methyl iodide (0.46 g, 0.20 ml, 3.29 mmol) was added. The reaction mixture was stirred at room temperature for 12 hours. The progress of the reaction was monitored by TLC. The reaction mixture was quenched with ice-water (5 ml) and extracted with ethyl acetate (2×20 ml). Organic layer washed with brine (10 ml). The organic layer separated was dried over anhydrous sodium sulphate and concentrated under reduced pressure to obtain a crude product. The crude product was further purified by column chromatography using 15% ethyl acetate in hexanes as an eluent to obtain the title compound (0.40 g, 73.5%). MS: m/z 347 (M+1).

¹H NMR (CDCl₃, 400 MHz): δ 7.85 (d, J=8.4 Hz, 2H), 7.43 (s, 1H), 7.40 (d, J=8.4 Hz, 2H), 3.98-4.01 (m, 1H), 3.34-3.39 (m, 1H), 3.14-3.18 (m, 1H), 2.86-2.94 (m, 1H), 1.74 (s, 3H), 1.71 (s, 3H), 1.29-1.37 (m, 2H), 0.46-0.51 (m, 1H), 0.01-0.02 (m, 1H).

The compound given below was prepared by procedure similar to the one described above for compound ‘13a’ with appropriate variations of reactants, reaction conditions and quantities of reagents.

14a. 2-(4-(4-chlorophenyl)thiazol-2-yl)-2-methyl-1-(pyrrolidin-1-yl)propan-1-one. MS: m/z 335 (M+1).

### Step 2: 4-(2-(1-(3-azabicyclo[3.1.0]hexan-3-yl)-2-methyl-1-oxopropan-2-yl)-4-(4-chlorophenyl)thiazol-5-yl)benzenesulfonamide (Compound 13)

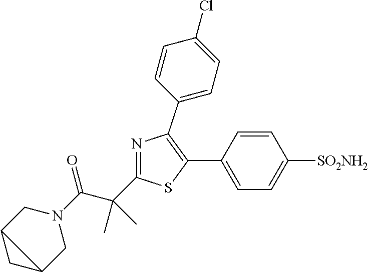 [SMILES: CC(C)(C(=O)N1CC2CC2C1)c1nc(-c2ccc(Cl)cc2)c(-c2ccc(S(N)(=O)=O)cc2)s1]

4-bromobenzenesulfonamide (0.32 g, 1.38 mmol) and potassium acetate (0.28 g, 2.88 mmol) were added to a solution of 1-(3-azabicyclo[3.1.0]hexan-3-yl)-2-(4-(4-chlorophenyl)thiazol-2-yl)-2-methylpropan-1-one (Compound 13a, 0.4 g, 1.15 mmol) in a DMA (5 ml) in a tube at 25° C. Nitrogen gas was bubbled through the reaction mixture for 15 minutes. Palladium acetate (0.013 g, 0.06 mmol) was then added to the reaction mixture under nitrogen atmosphere and the tube was sealed. The reaction mixture was heated at 150° C. for 20 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 5% methanol in DCM as an eluent to obtain the title compound (0.22 g, 38.9%). MS: m/z 503 (M+1).

¹H NMR (DMSO-d₆, 400 MHz): δ 7.82 (d, J=8.4 Hz, 2H), 7.55 (d, J=8.4 Hz, 2H), 7.46 (bs-exchanges with D₂O, 2H), 7.42 (s, 4H), 3.77 (d, J=11.2 Hz, 1H), 3.12-3.33 (m, 3H), 1.60 (s, 3H), 1.57 (s, 3H), 1.42-1.45 (m, 2H), 0.48-0.52 (m, 1H), 0.01-0.02 (m, 1H).

The following compound was prepared according to the procedure described above for compound 13, with appropriate changes to the reactants.

4-(4-(4-chlorophenyl)-2-(2-methyl-1-oxo-1-(pyrrolidin-1-yl)propan-2-yl)thiazol-5-yl)benzenesulfonamide (Compound 14). MS: m/z 490 (M+1).

¹H NMR (DMSO-d₆, 400 MHz): δ 7.82 (d, J=8.4 Hz, 2H), 7.54 (d, J=8.4 Hz, 2H), 7.45 (bs-exchanges with D₂O, 2H), 7.40-7.42 (s, 4H), 3.30-3.48 (m, 2H), 2.95-3.04 (m, 2H), 1.68-1.1.80 (m, 4H), 1.63 (s, 6H).

### Example 7

### 4-(2-(1-(3-azabicyclo[3.1.0]hexane-3-carbonyl)cyclopropyl)-4-(4-chlorophenyl)thiazol-5-yl)benzenesulfonamide (Compound 15)

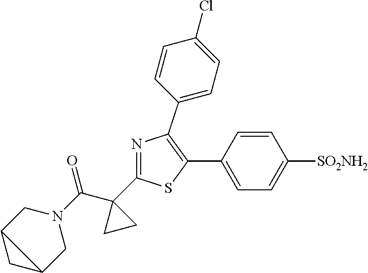 [SMILES: NS(=O)(=O)c1ccc(-c2sc(C3(C(=O)N4CC5CC5C4)CC3)nc2-c2ccc(Cl)cc2)cc1]

### Step 1: 3-azabicyclo[3.1.0]hexan-3-yl(1-(4-(4-chlorophenyl)thiazol-2-yl)cyclopropyl)methanone (15a)

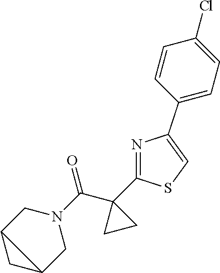 [SMILES: O=C(N1CC2CC2C1)C1(c2nc(-c3ccc(Cl)cc3)cs2)CC1]

Sodium ethoxide (0.23 g, 3.45 mmol) was added to a solution of 1-(3-azabicyclo[3.1.0]hexan-3-yl)-2-(4-(4-chlorophenyl)thiazol-2-yl) ethanone (Compound 10a, 0.5 g, 1.57 mmol) in DMF (10 ml) under nitrogen atmosphere with stirring at 0° C. To the reaction mixture 1,2-dibromoethane (0.32 g, 0.15 ml, 1.72 mmol) was added. The reaction mixture was stirred at room temperature for 15 hours. The progress of the reaction was monitored by TLC. The reaction mixture was quenched with ice-water (5 ml) and extracted with ethyl acetate (2×20 ml). Organic layer washed with brine (10 ml). The organic layer separated was dried over anhydrous sodium sulphate and concentrated under reduced pressure to obtain a crude product. The crude product was further purified by column chromatography using 15% ethyl acetate in hexanes as an eluent to obtain the title compound (0.30 g, 55.5%). MS: m/z 345 (M+1).

¹H NMR (CDCl₃, 400 MHz): δ 7.82 (d, J=8.4 Hz, 2H), 7.37 (d, J=8.4 Hz, 2H), 7.33 (s, 1H), 3.97 (d, J=12.0 Hz, 1H), 3.72 (d, J=10.4 Hz, 1H), 3.38-3.45 (m, 2H), 1.46-1.60 (m, 6H), 0.63-0.68 (m, 1H), 0.14-0.18 (m, 1H).

The compound given below was prepared by procedure similar to the one described above for compound ‘15a’ with appropriate variations of reactants, reaction conditions and quantities of reagents.

16a. (1-(4-(4-chlorophenyl)thiazol-2-yl)cyclopropyl) (pyrrolidin-1-yl)methanone. MS: m/z 333 (M+1).

### Step 2: 4-(2-(1-(3-azabicyclo[3.1.0]hexane-3-carbonyl)cyclopropyl)-4-(4-chlorophenyl)thiazol-5-yl)benzenesulfonamide (Compound 15)

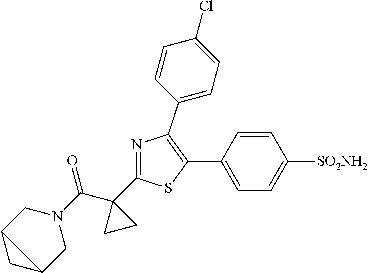 [SMILES: NS(=O)(=O)c1ccc(-c2sc(C3(C(=O)N4CC5CC5C4)CC3)nc2-c2ccc(Cl)cc2)cc1]

4-bromobenzenesulfonamide (0.33 g, 1.39 mmol) and potassium acetate (0.28 g, 2.90 mmol) were added to a solution of 3-azabicyclo[3.1.0]hexan-3-yl(1-(4-(4-chlorophenyl)thiazol-2-yl)cyclopropyl)methanone (Compound 15a, 0.40 g, 1.16 mmol) in a DMA (5 ml) in a tube at 25° C. Nitrogen gas was bubbled through the reaction mixture for 15 minutes. Palladium acetate (0.013 g, 0.06 mmol) was then added to the reaction mixture under nitrogen atmosphere and the tube was sealed. The reaction mixture was heated at 150° C. for 20 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 5% methanol in DCM as an eluent to obtain the title compound (0.12 g, 19.8%). MS: m/z 501 (M+1).

¹H NMR (DMSO-d₆, 400 MHz): δ 7.80 (d, J=8.0 Hz, 2H), 7.50 (d, J=8.0 Hz, 2H), 7.40-7.45 (m, 6H), 3.65-3.75 (m, 2H), 3.46-3.49 (m, 2H), 1.44-1.65 (m, 6H), 0.63-0.64 (m, 1H), 0.11-0.14 (m, 1H).

The following compound was prepared according to the procedure described above for compound 15, with appropriate changes to the reactants.

4-(4-(4-chlorophenyl)-2-(1-(pyrrolidine-1-carbonyl)cyclopropyl)thiazol-5-yl)benzenesulfonamide (Compound 16). MS: m/z 488 (M+1).

¹H NMR (DMSO-d₆, 400 MHz): δ 7.80 (d, J=8.4 Hz, 2H), 7.52 (d, J=8.4 Hz, 2H), 7.45 (bs-exchanges with D₂O, 2H), 7.40-7.43 (s, 4H), 3.33-3.48 (m, 4H), 2.72-2.88 (m, 4H), 1.45-1.54 (m, 2H), 1.56-1.60 (m, 2H).

### Example 8

### 4-(2-(2-(3-azabicyclo[3.1.0]hexan-3-yl)-1,1-difluoro-2-oxoethyl)-4-(4-chlorophenyl)thiazol-5-yl)benzenesulfonamide (Compound 17)

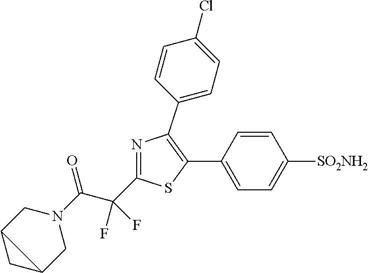 [SMILES: NS(=O)(=O)c1ccc(-c2sc(C(F)(F)C(=O)N3CC4CC4C3)nc2-c2ccc(Cl)cc2)cc1]

### Step 1: Methyl 2-(4-bromothiazol-2-yl)-2-oxoacetate (17a)

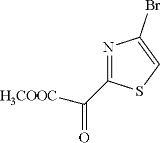

A solution of 4-bromo-2-(trimethylsilyl)thiazole (prepared according to the procedure reported by A. Dondoni et al., Synthesis, 1986, 9, 757-760) (18.00 g, 76.00 mmol) in DCM (180 ml) was added methyl oxalyl chloride (9.34 g, 7.07 ml, 76.00 mmol). The reaction mixture was stirred at room temperature for 18 hrs. The progress of the reaction was monitored by TLC. The reaction mixture was quenched with saturated sodium bicarbonate solution (50 ml). The mixture was then extracted with DCM (2×100 ml). The combined organic layer was dried over anhydrous Na₂SO₄. The solvent was evaporated from the dried organic layer under reduced pressure to obtain a crude product; the crude product was further purified by column chromatography using 15% ethyl acetate in hexanes as an eluent to obtain the title compound (10.10 g, 53%). MS: m/z 251 (M+1).

¹H NMR (CDCl₃, 400 MHz): δ 7.76 (s, 1H), 4.03 (s, 3H).

### Step 2: Methyl 2-(4-bromothiazol-2-yl)-2,2-difluoroacetate (17b)

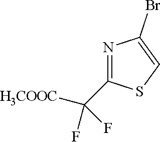

Diethylamino sulfur trifluoride (4.40 g, 3.6 ml, and 27.30 mmol) was added to a solution of methyl 2-(4-bromothiazol-2-yl)-2-oxoacetate (Compound 17a, 3.1 g, 12.40 mmol) in DCM (10 ml) at 0° C. The reaction mixture was stirred at 25° C. for 6 hrs. The progress of the reaction was monitored by TLC. The reaction mixture was quenched with saturated sodium bicarbonate solution (20 ml). The mixture was then extracted with ethyl acetate (2×50 ml). The combined organic layer was dried over anhydrous Na₂SO₄. The solvent was evaporated from the dried organic layer under reduced pressure to obtain a crude product; the crude product was further purified by flash column chromatography using 10% ethyl acetate in hexanes as an eluent to obtain the title compound (2.71 g, 80%). MS: m/z 273 (M+1).

¹H NMR (CDCl₃, 400 MHz): δ 7.49 (s, 1H), 3.97 (s, 3H).

### Step 3: 1-(3-azabicyclo[3.1.0]hexan-3-yl)-2-(4-bromothiazol-2-yl)-2,2-difluoroethanone (17c)

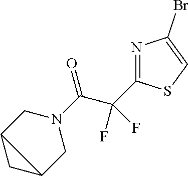 [SMILES: O=C(N1CC2CC2C1)C(F)(F)c1nc(Br)cs1]

(1R,5S)-3-azabicyclo[3.1.0]hexane hydrochloride (0.44 g, 3.68 mmol) and triethyl amine (1.48 g, 2.05 ml, 14.70 mmol) were added to a solution of methyl 2-(4-bromothiazol-2-yl)-2,2-difluoroacetate (Compound 17b, 1.0 g, 3.68 mmol) in DCM (20 ml) at 25° C. The reaction mixture was stirred at 25° C. for 18 hrs. The progress of the reaction was monitored by TLC. The solvent was evaporated from the reaction mixture under reduced pressure to obtain a crude product; the crude product was further purified by flash column chromatography using 20% ethyl acetate in hexanes as an eluent to obtain the title compound (0.92 g, 77%). MS: m/z 324 (M+1).

¹H NMR (CDCl₃, 400 MHz): δ 7.48 (s, 1H), 3.95-4.01 (m, 2H), 3.73 (dd, J=11.2, 4.0 Hz, 1H), 3.55 (dd, J=12.0, 4.0 Hz, 1H), 1.54-1.72 (m, 2H), 0.72-0.77 (m, 1H), 0.25-0.28 (m, 1H).

The compound given below was prepared by procedure similar to the one described above for compound ‘17c’ with appropriate variations of reactants, reaction conditions and quantities of reagents.

18c. 2-(4-bromothiazol-2-yl)-2,2-difluoro-1-(pyrrolidin-1-yl)ethanone. MS: m/z 310 (M+1).

### Step 4: 1-(3-azabicyclo[3.1.0]hexan-3-yl)-2-(4-(4-chlorophenyl)thiazol-2-yl)-2,2-difluoroethanone (17d)

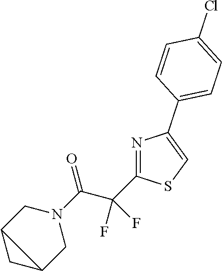 [SMILES: O=C(N1CC2CC2C1)C(F)(F)c1nc(-c2ccc(Cl)cc2)cs1]

(4-chlorophenyl) boronic acid (0.48 g, 3.06 mmol) and potassium carbonate (0.96 g, 6.96 mmol) were added to a solution of 1-(3-azabicyclo[3.1.0]hexan-3-yl)-2-(4-bromothiazol-2-yl)-2,2-difluoroethanone (Compound 17c, 0.9 g, 2.79 mmol) in a mixture of toluene:ethanol (5:15 ml) in a tube at 25° C. Nitrogen gas was bubbled through the reaction mixture for 15 minutes. Tetrakis (triphenylphosphine) (0) palladium (0.16 g, 0.14 mmol) was then added to the reaction mixture under nitrogen atmosphere and the tube was sealed. The reaction mixture was heated at 95-100° C. for 18 hr under stirring. The progress of the reaction was monitored by TLC. The reaction mixture was then cooled to 25° C. and filtered through celite. The celite cake was washed with 10% methanol in DCM (30 ml). The combined filtrate was concentrated under reduced pressure to obtain a crude product, which was then purified by column chromatography using 30% ethyl acetate in hexanes as an eluent to obtain the title compound (0.73 g, 73.9%). MS: m/z 356 (M+1).

¹H NMR (CDCl₃, 400 MHz): δ 7.82 (d, J=8.8 Hz, 2H), 7.68 (s, 1H), 7.41 (d, J=8.8 Hz, 2H), 4.09-4.14 (m, 1H), 4.03 (d, J=12.4 Hz, 1H), 3.74 (dd, J=10.8, 4.0 Hz, 1H), 3.57 (dd, J=12.0, 4.0 Hz, 1H), 1.55-1.65 (m, 2H), 0.70-0.75 (m, 1H), 0.25-0.27 (m, 1H).

The compound given below was prepared by procedure similar to the one described above for compound ‘17d’ with appropriate variations of reactants, reaction conditions and quantities of reagents.

18d. 2-(4-(4-chlorophenyl)thiazol-2-yl)-2,2-difluoro-1-(pyrrolidin-1-yl)ethanone. MS: m/z 343 (M+1).

### Step 5: 4-(2-(2-(3-azabicyclo[3.1.0]hexan-3-yl)-1,1-difluoro-2-oxoethyl)-4-(4-chlorophenyl)thiazol-5-yl)benzenesulfonamide (Compound 17)

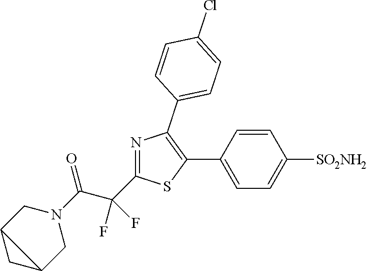 [SMILES: NS(=O)(=O)c1ccc(-c2sc(C(F)(F)C(=O)N3CC4CC4C3)nc2-c2ccc(Cl)cc2)cc1]

4-bromobenzenesulfonamide (0.51 g, 2.17 mmol) and potassium acetate (0.38 g, 3.95 mmol) were added to a solution of 1-(3-azabicyclo[3.1.0]hexan-3-yl)-2-(4-(4-chlorophenyl)thiazol-2-yl)-2,2-difluoroethanone (Compound 17d, 0.70 g, 1.97 mmol) in a DMA (15 ml) in a tube at 25° C. Nitrogen gas was bubbled through the reaction mixture for 15 minutes. Palladium acetate (0.04 g, 0.19 mmol) was then added to the reaction mixture under nitrogen atmosphere and the tube was sealed. The reaction mixture was heated at 150° C. for 18 hrs under stirring. The progress of the reaction was monitored by TLC. The reaction mixture was then cooled to 25° C. and filtered through celite. The celite cake was washed with ethyl acetate (30 ml). The combined filtrate was concentrated under reduced pressure to obtain a crude product, which was then purified by column chromatography using 30% ethyl acetate in hexanes as an eluent to obtain the title compound (0.23 g, 23.4%). MS: m/z 511 (M+1).

¹H NMR (DMSO-d₆, 400 MHz): δ 7.86 (d, J=8.4 Hz, 2H), 7.64 (d, J=8.4 Hz, 2H), 7.40-7.50 (m, 6H), 3.80-3.86 (m, 2H), 3.68 (dd, J=10.8, 4.0 Hz, 1H), 3.51 (dd, J=12.0, 4.0 Hz, 1H), 1.57-1.66 (m, 2H), 0.66-0.70 (m, 1H), 0.07-0.11 (m, 1H).

The following compound was prepared according to the procedure described above for compound 17, with appropriate changes to the reactants.

4-(4-(4-chlorophenyl)-2-(1,1-difluoro-2-oxo-2-(pyrrolidin-1-yl)ethyl)thiazol-5-yl)benzenesulfonamide (Compound 18). MS: m/z 499 (M+1).

¹H NMR (DMSO-d₆, 400 MHz): δ 7.85 (d, J=8.0 Hz, 2H), 7.64 (d, J=8.0 Hz, 2H), 7.41-7.50 (m, 6H), 3.67 (t, J=6.8 Hz, 2H), 3.49 (t, J=6.8 Hz, 2H), 1.81-1.95 (m, 4H).

### Example 9

### 4-(4-(4-chlorophenyl)-2-(6-fluoropyridin-2-yl)thiazol-5-yl)benzenesulfonamide (Compound 19)

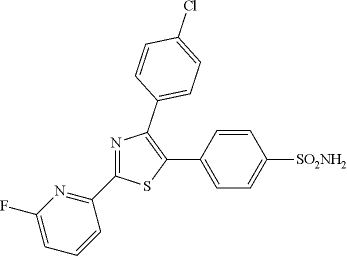 [SMILES: NS(=O)(=O)c1ccc(-c2sc(-c3cccc(F)n3)nc2-c2ccc(Cl)cc2)cc1]

### Step 1: 4-bromo-2-(6-fluoropyridin-2-yl)thiazole (19a)

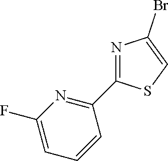 [SMILES: Fc1cccc(-c2nc(Br)cs2)n1]

(6-fluoropyridin-2-yl) boronic acid (3.0 g, 21.29 mmol) and potassium carbonate (8.17 g, 59.1 mmol) were added to a solution of 2,4-dibromothiazole (5.75 g, 23.66 mmol) in ethanol (50 ml) and toluene (10 ml) at 25° C. Nitrogen gas was bubbled through the reaction mixture for 15 minutes. Tetrakis(triphenylphosphine) palladium(0) (1.367 g, 1.183 mmol) was then added to the reaction mixture under nitrogen atmosphere. The reaction mixture was heated at 100° C. for 3 hr. under stirring. The progress of the reaction was monitored by TLC. The reaction mixture was then cooled to 25° C. and filtered through celite. The celite cake was washed with ethyl acetate (50 ml). The combined filtrate was concentrated under reduced pressure to obtain a crude product, which was then purified by column chromatography using 5% ethyl acetate in hexanes as an eluent to obtain the title compound (1.4 g, 22.84%). MS: m/z 260 (M+1).

¹H NMR (CDCl₃, 400 MHz): δ8.08-8.11 (m, 1H), 7.89-7.95 (m, 1H), 7.38 (s, 1H), 7.00-7.03 (m, 1H).

### Step 2: 4-(4-chlorophenyl)-2-(6-fluoropyridin-2-yl)thiazole (19b)

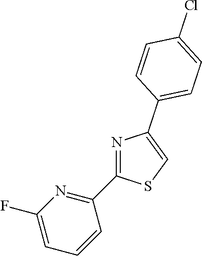 [SMILES: Fc1cccc(-c2nc(-c3ccc(Cl)cc3)cs2)n1]

(4-Chlorophenyl)boronic acid (0.845 g, 5.40 mmol) and potassium carbonate (1.87 g, 13.53 mmol) were added to a solution of 4-bromo-2-(6-fluoropyridin-2-yl)thiazole (Compound 19a, 1.4 g, 5.40 mmol) in ethanol (30 ml) and toluene (10 ml) at 25° C. Nitrogen gas was bubbled through the reaction mixture for 15 minutes. tetrakis(triphenylphosphine) palladium(0) (0.312 g, 0.270 mmol) was then added to the reaction mixture under nitrogen atmosphere. The reaction mixture was heated at 100° C. for 3 hr. under stirring. The progress of the reaction was monitored by TLC. The reaction mixture was then cooled to 25° C. and filtered through celite. The celite cake was washed with ethyl acetate (30 ml). The combined filtrate was concentrated under reduced pressure to obtain a crude product, which was then purified by column chromatography using 5% ethyl acetate in hexanes as an eluent to obtain the title compound (1.5 g, 95%)

¹H NMR (CDCl₃, 400 MHz): δ 8.21 (dd, J=7.6, 2.0 Hz, 1H), 7.91-7.97 (m, 3H), 7.63 (s, 1H), 7.42-7.45 (m, 2H), 7.01 (dd, J=7.6 Hz, 2.0 Hz, 1H).

### Step 3: 4-(4-(4-chlorophenyl)-2-(6-fluoropyridin-2-yl)thiazol-5-yl)benzenesulfonamide (Compound 19)

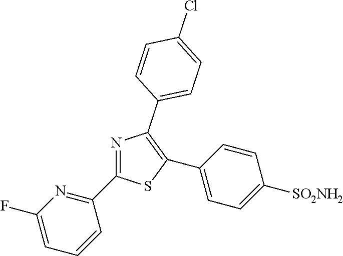 [SMILES: NS(=O)(=O)c1ccc(-c2sc(-c3cccc(F)n3)nc2-c2ccc(Cl)cc2)cc1]

4-bromobenzenesulfonamide (0.68 g, 2.88 mmol) and potassium acetate (0.514 g, 5.24 mmol) were added to a solution of 4-(4-chlorophenyl)-2-(6-fluoropyridin-2-yl)thiazole (Compound 19b, 1.5 g, 5.16 mmol) in a DMA (5 ml) in a tube at 25° C. Nitrogen gas was bubbled through the reaction mixture for 15 minutes. Palladium acetate (0.059 g, 0.262 mmol) was then added to the reaction mixture under nitrogen atmosphere and the tube was sealed. The reaction mixture was heated at 135° C. for 10 hr. under stirring. The progress of the reaction was monitored by TLC. The reaction mixture was then cooled to 25° C. and concentrated under reduced pressure to remove solvent; residue was diluted with ethyl acetate (20 ml) and filtered through celite. The celite cake was washed with ethyl acetate (20 ml). The combined filtrate was concentrated under reduced pressure to obtain a crude product, which was then purified by column chromatography using 30% ethyl acetate in hexanes as an eluent to obtain the title compound (0.35 g, 30.0%). MS: m/z 446.2 (M+1).

¹H NMR (CDCl₃, 400 MHz): δ 8.12-8.21 (m, 2H), 7.83 (d, J=8.4 Hz, 2H), 7.58 (d, J=8.4 Hz, 2H), 7.43-7.51 (m, 6H), 7.31-7.33 (m, 1H).

The following compounds were prepared according to the procedure described above for compound 19, with appropriate changes to the reactants.

4-(4-(4-chlorophenyl)-2-(pyridin-3-yl)thiazol-5-yl)benzenesulfonamide (Compound 20). MS: m/z 429 (M+1).

¹H NMR (DMSO-d₆, 400 MHz): δ 9.20 (d, J=2.0 Hz, 1H), 8.73 (dd, J=4.8, 1.6 Hz, 1H), 8.38 (dd, J=8.0, 1.6 Hz, 1H), 7.86 (d, J=8.4 Hz, 2H), 7.57-7.60 (m, 3H), 7.51 (d, J=8.4 Hz, 2H), 7.47-7.49 (m, 4H).

4-(4-(4-chlorophenyl)-2-(pyridin-4-yl)thiazol-5-yl)benzenesulfonamide (Compound 21). MS: m/z 429 (M+1).

¹H NMR (DMSO-d₆, 400 MHz): δ 8.77 (dd, J=8.4, 1.6 Hz, 2H), 7.96 (dd, J=8.4, 1.6 Hz, 2H), 7.86 (d, J=8.4, 2H), 7.62 (d, J=8.4 Hz, 2H), 7.53-7.55 (m, 2H), 7.47-7.49 (m, 4H).

4-(4-(4-chlorophenyl)-2-(pyridin-2-yl)thiazol-5-yl)benzenesulfonamide (Compound 22). MS: m/z 429 (M+1).

¹H NMR (DMSO-d₆, 400 MHz): δ 8.67 (d, J=8.4 Hz, 1H), 8.22 (d, J=8.0 Hz, 1H), 8.02 (td, J=7.6, 1.6 Hz, 1H), 7.85 (d, J=8.4 Hz, 2H), 7.61 (d, J=8.4 Hz, 2H), 7.53-7.58 (m, 3H), 7.48-7.49 (m, 4H).

4-(4-(4-chlorophenyl)-2-(pyridin-2-yl)thiazol-5-yl)-3-fluorobenzenesulfonamide (Compound 23). MS: m/z 446 (M+1).

¹H NMR (DMSO-d₆, 400 MHz): δ 8.68 (d, J=4.0 Hz, 1H), 8.24 (dt, J=8.0, 1.6 Hz, 1H), 8.04 (t, J=8.4 Hz, 1H), 7.71-7.76 (m, 3H), 7.63 (bs, exchanges with D₂O, 2H), 7.52-7.59 (m, 3H), 7.46-7.48 (m, 2H).

4-(4-(4-chlorophenyl)-2-phenylthiazol-5-yl)benzenesulfonamide (Compound 24). MS: m/z 427 (M+1).

¹H NMR (DMSO-d₆, 400 MHz): δ 8.01-8.03 (m, 2H), 7.84 (d, J=8.4 Hz, 2H), 7.59 (d, J=8.4 Hz, 2H), 7.53-7.57 (m, 5H), 7.46-7.48 (m, 4H).

4-(4-(4-chlorophenyl)-2-(5-fluoropyridin-2-yl)thiazol-5-yl)benzenesulfonamide (Compound 25). MS: m/z 446 (M+1).

¹H NMR (DMSO-d₆, 400 MHz): δ 8.71 (d, J=2.8 Hz, 1H), 8.28-8.31 (m, 1H), 7.96 (dt, J=8.4, 2.8 Hz, 1H), 7.85 (d, J=8.4 Hz, 2H), 7.61 (d, J=8.4 Hz, 2H), 7.54 (d, J=8.8 Hz, 2H), 7.48-7.49 (m, 4H).

### Example 10

### 4-(4-chlorophenyl)-N-cyclopropyl-N-methyl-5-(4-sulfamoylphenyl)thiazole-2-carboxamide (Compound 26)

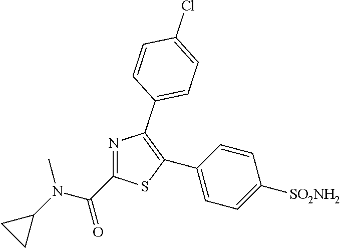 [SMILES: CN(C(=O)c1nc(-c2ccc(Cl)cc2)c(-c2ccc(S(N)(=O)=O)cc2)s1)C1CC1]

### Step 1: 4-(4-chlorophenyl)-N-cyclopropyl-N-methylthiazole-2-carboxamide (26a)

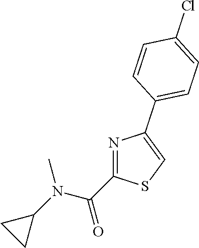 [SMILES: CN(C(=O)c1nc(-c2ccc(Cl)cc2)cs1)C1CC1]

Sodium hydride (0.068 g, 60% dispersion in paraffin oil, 1.72 mmol) was added to a solution of 4-(4-chlorophenyl)-N-cyclopropylthiazole-2-carboxamide (Prepared according to the procedure reported in US 2009/318429, 0.40 g, 1.43 mmol) in DMF (8 ml) at 0° C. To this reaction mixture methyl iodide (0.30 g, 0.13 ml, 2.15 mmol) was added at 0° C. The reaction mixture was stirred at 25° C. for 6.0 hr. The progress of the reaction was monitored by TLC. The reaction mixture was quenched with water (10 ml). The mixture was then extracted with ethyl acetate (2×25 ml). The combined organic layer was dried over anhydrous Na₂SO₄. The solvent was evaporated from the dried organic layer under reduced pressure to obtain a crude product; the crud product was further purified by flash column chromatography using 20% ethyl acetate in hexanes as an eluent to obtain the title compound (0.38 g, 90.4%). MS: m/z 293 (M+1).

¹H NMR (CDCl₃, 400 MHz): δ 7.84 (d, J=8.4 Hz, 2H), 7.66 (s, 1H), 7.41 (d, J=8.4 Hz, 2H), 3.55-3.62 (m, 1H), 3.18 (s, 3H), 0.68-0.92 (m, 4H).

The compounds given below were prepared by procedure similar to the one described above for compound ‘26a’ with appropriate variations of reactants, reaction conditions and quantities of reagents.

28a. 4-(4-chlorophenyl)-N-cyclopentyl-N-methylthiazole-2-carboxamide. MS: m/z 321 (M+1).

29a. 4-(4-chlorophenyl)-N-cyclohexyl-N-methylthiazole-2-carboxamide. MS: m/z 335 (M+1).

30a. 4-(4-chlorophenyl)-N-(cyclopropylmethyl)-N-methylthiazole-2-carboxamide. MS: m/z 307 (M+1).

### Step 2: 4-(4-chlorophenyl)-N-cyclopropyl-N-methyl-5-(4-sulfamoylphenyl)thiazole-2-carboxamide (Compound 26)

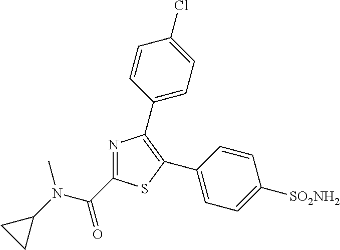 [SMILES: CN(C(=O)c1nc(-c2ccc(Cl)cc2)c(-c2ccc(S(N)(=O)=O)cc2)s1)C1CC1]

4-bromobenzene sulfonamide (0.36 g, 1.53 mmol) and potassium acetate (0.37 g, 3.84 mmol) were added to a solution of 4-(4-chlorophenyl)-N-cyclopropyl-N-methylthiazole-2-carboxamide (Compound 26a, 0.45 g, 1.53 mmol) in a DMA (5 ml) in a tube at 25° C. Nitrogen gas was bubbled through the reaction mixture for 15 minutes. Palladium acetate (0.035 g, 0.15 mmol) was then added to the reaction mixture under nitrogen atmosphere and the tube was sealed. The reaction mixture was heated at 150° C. for 8 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 55% ethyl acetate in hexanes as an eluent to obtain the title compound (0.14 g, 20.33%). MS: m/z 448 (M+1).

¹H NMR (CDCl₃, 400 MHz): δ 7.93 (d, J=8.4 Hz, 2H), 7.53 (d, J=8.4 Hz, 2H), 7.41 (d, J=8.4 Hz, 2H), 7.30 (d, J=8.4 Hz, 2H), 4.93 (bs-exchanges with D₂O, 2H), 3.54-3.59 (m, 1H), 3.18 (s, 3H), 0.73-0.91 (m, 4H).

The following compounds were prepared according to the procedure described above for compound 26, with appropriate changes to the reactants.

4-(4-chlorophenyl)-N-cyclopropyl-5-(2-fluoro-4-sulfamoylphenyl)-N-methylthiazole-2-carboxamide (Compound 27). MS: m/z 466 (M+1).

¹H NMR (DMSO-d₆, 400 MHz): δ 7.70-7.77 (m, 3H), 7.63 (bs, exchanges with D₂O, 2H), 7.43-7.48 (m, 4H), 3.48 (s, 1H), 3.16 (s, 3H), 0.81-0.82 (m, 2H), 0.72-0.77 (m, 2H).

4-(4-chlorophenyl)-N-cyclopentyl-N-methyl-5-(4-sulfamoylphenyl)thiazole-2-carboxamide (Compound 28). MS: m/z 476 (M+1).

¹H NMR (DMSO-d₆, 400 MHz): δ 7.85 (d, J=8.4 Hz, 2H), 7.60 (d, J=8.4 Hz, 2H), 7.49 (m, 6H), 5.47-5.49 (m, 1H), 3.33 (s, 3H), 1.48-1.85 (m, 6H), 1.35-1.37 (m, 2H).

4-(4-chlorophenyl)-N-cyclohexyl-N-methyl-5-(4-sulfamoylphenyl)thiazole-2-carboxamide (Compound 29). MS: m/z 490 (M+1).

¹H NMR (DMSO-d₆, 400 MHz): δ 7.85 (d, J=8.4 Hz, 2H), 7.60 (d, J=8.4 Hz, 2H), 7.48 (bs-exchanges with D₂O, 2H), 7.42-7.47 (m, 4H), 3.40-3.46 (m, 1H), 2.95-3.02 (m, 2H), 1.72-1.86 (m, 3H), 1.52-1.70 (m, 4H), 1.18-1.42 (m, 2H), 1.05-1.16 (m, 1H), 0.96-1.02 (m, 1H).

44-(4-chlorophenyl)-N-(cyclopropylmethyl)-N-methyl-5-(4-sulfamoylphenyl)thiazole-2-carboxamide (Compound 30). MS: m/z 462 (M+1).

¹H NMR (CDCl₃, 400 MHz): δ 7.93 (d, J=8.4 Hz, 2H), 7.52 (d, J=8.4 Hz, 2H), 7.31-7.44 (m, 4H), 4.92 (bs-exchanges with D₂O, 2H), 4.07 (d, J=7.2 Hz, 1H), 3.70-3.74 (m, 1H), 3.50 (d, J=7.2 Hz, 1H), 3.22-3.26 (m, 1H), 1.20-1.34 (m, 2H), 0.56-0.62 (m, 2H), 0.33-0.34 (m, 2H).

### Example 11

### 4-(4-chlorophenyl)-N-cyclohexyl-5-(4-sulfamoylphenyl)thiazole-2-carboxamide (Compound 31)

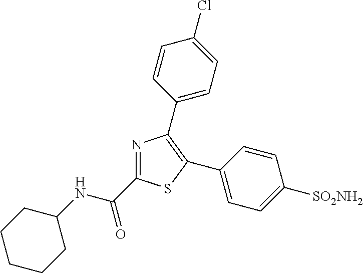 [SMILES: NS(=O)(=O)c1ccc(-c2sc(C(=O)NC3CCCCC3)nc2-c2ccc(Cl)cc2)cc1]

4-bromobenzene sulfonamide (0.40 g, 1.71 mmol) and potassium acetate (0.30 g, 3.12 mmol) were added to a solution of 4-(4-chlorophenyl)-N-cyclohexylthiazole-2-carboxamide (Prepared according to the procedure reported in US 2009/318429, 0.50 g, 1.56 mmol) in a DMA (5 ml) in a tube at 25° C. Nitrogen gas was bubbled through the reaction mixture for 15 minutes. Palladium acetate (0.035 g, 0.156 mmol) was then added to the reaction mixture under nitrogen atmosphere and the tube was sealed. The reaction mixture was heated at 150° C. for 20 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 30% ethyl acetate in hexanes as an eluent to obtain the title compound (0.18 g, 24.2%). MS: m/z 477 (M+1).

¹H NMR (DMSO-d₆, 400 MHz): δ 8.71 (d-exchanges with D₂O, J=8.4 Hz, 1H), 7.83 (d, J=8.4 Hz, 2H), 7.58 (d, J=8.4 Hz, 2H), 7.52 (d, J=8.4, 2H), 7.45-7.47 (m, 4H), 3.73-3.81 (m, 1H), 1.59-1.81 (m, 5H), 1.15-1.50 (m, 5H).

The following compounds were prepared according to the procedure described above for compound 31, with appropriate changes to the reactants.

4-(4-chlorophenyl)-N,N-dipropyl-5-(4-sulfamoylphenyl)thiazole-2-carboxamide (Compound 32). MS: m/z 478 (M+1).

¹H NMR (DMSO-d₆, 400 MHz): δ 7.84 (d, J=8.4 Hz, 2H), 7.60 (d, J=8.4 Hz, 2H), 7.48 (bs-exchanges with D₂O, 2H), 7.42-7.46 (m, 4H), 3.91-4.01 (m, 2H), 3.40-3.48 (m, 2H), 1.57-1.68 (m, 4H), 0.82-0.93 (m, 6H).

4-(4-chlorophenyl)-N-(2-hydroxyethyl)-N-propyl-5-(4-sulfamoylphenyl)thiazole-2-carboxamide (Compound 33). MS: m/z 480 (M+1).

¹H NMR (DMSO-d₆, 400 MHz): δ 7.85 (d, J=8.4 Hz, 2H), 7.60 (d, J=8.4 Hz, 2H), 7.48 (bs-exchanges with D₂O, 2H), 7.40-7.45 (m, 4H), 4.72-4.84 (bs-exchanges with D₂O, 1H), 3.96-4.15 (m, 2H), 3.58-3.73 (m, 2H), 3.45-3.58 (m, 2H), 1.57-1.72 (m, 2H), 0.71-0.92 (m, 3H).

### Example 12

### N-(4-(4-chlorophenyl)-5-(4-sulfamoylphenyl)thiazol-2-yl)-N-ethylcyclopropanecarboxamide (Compound 34)

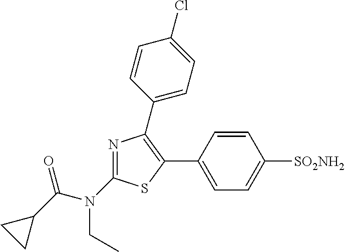 [SMILES: CCN(C(=O)C1CC1)c1nc(-c2ccc(Cl)cc2)c(-c2ccc(S(N)(=O)=O)cc2)s1]

### Step 1: N-(4-(4-chlorophenyl)thiazol-2-yl)cyclopropanecarboxamide (34a)

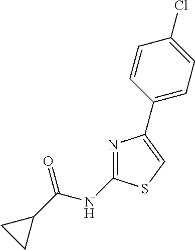 [SMILES: O=C(Nc1nc(-c2ccc(Cl)cc2)cs1)C1CC1]

DMAP (0.17 g, 1.42 mmol) and cyclopropanes carbonyl chloride (0.74 g, 0.64 ml, 7.12 mmol) were added to a solution of 4-(4-chlorophenyl)thiazol-2-amine (Prepared according to the procedure reported by G. L. Talesara et al., Journal of Indian chemical society, 2008, 85, 660-664) 1.0 g, 4.75 mmol) in DMA (10 ml) at 25° C. The reaction mixture was stirred at 75° C. for 3 hr. The progress of the reaction was monitored by TLC. The solvent was evaporated under reduced pressure to obtain solid residue. The residue was dissolved in ethyl acetate (30 ml) and washed with saturated sodium bicarbonate solution (20 ml). The organic layer was dried over anhydrous Na₂SO₄. The solvent was evaporated under reduced pressure to obtain a crude product; the crude product was further purified by flash column chromatography using 15% ethyl acetate in hexanes as an eluent to obtain the title compound (0.90 g, 68%). MS: m/z 279 (M+1).

¹H NMR (CDCl₃, 400 MHz): δ 11.22 (bs-exchanges with D₂O, 1H), 7.75 (d, J=8.8 Hz, 2H), 7.40 (d, J=8.8 Hz, 2H), 7.12 (s, 1H), 1.25-1.31 (m, 1H), 1.05-1.12 (m, 2H), 0.65-0.70 (m, 2H).

The compounds given below were prepared by procedure similar to the one described above for compound ‘34a’ with appropriate variations of reactants, reaction conditions and quantities of reagents.

36a. N-(4-(4-chlorophenyl)thiazol-2-yl)cyclopentane carboxamide. MS: m/z 307 (M+1).

37a. N-(4-(4-chlorophenyl)thiazol-2-yl)cyclohexanecarboxamide. MS: m/z 321 (M+1).

38a. N-(4-(4-chlorophenyl)thiazol-2-yl)propionamide. MS: m/z 267 (M+1).

39a. N-(4-(4-chlorophenyl)thiazol-2-yl)-2-methoxyacetamide. MS: m/z 283 (M+1).

### Step 2: N-(4-(4-chlorophenyl)thiazol-2-yl)-N-ethylcyclopropanecarboxamide (34b)

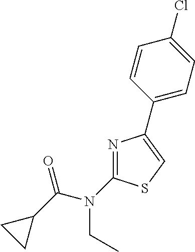 [SMILES: CCN(C(=O)C1CC1)c1nc(-c2ccc(Cl)cc2)cs1]

Sodium hydride (0.051 g, 60% dispersion in paraffin oil, 1.29 mmol) was added to a solution of N-(4-(4-chlorophenyl)thiazol-2-yl)cyclopropanecarboxamide (Compound 34a, 0.30 g, 1.07 mmol) in DMF (10 ml) at 0° C. To this reaction mixture ethyl iodide (0.17 g, 0.09 ml, 1.07 mmol) was added at 0° C. The reaction mixture was stirred at 25° C. for 0.5 hr. The progress of the reaction was monitored by TLC. The reaction mixture was quenched with water (10 ml). The mixture was then extracted with ethyl acetate (2×25 ml). The combined organic layer was dried over anhydrous Na₂SO₄. The solvent was evaporated under reduced pressure to obtain the title compound (0.19 g, 57%). MS: m/z 308 (M+1).

¹H NMR (CDCl₃, 400 MHz): δ 7.84 (d, J=8.8 Hz, 2H), 7.39 (d, J=8.8 Hz, 2H), 7.15 (s, 1H), 4.57 (q, J=6.8 Hz, 2H), 2.02-2.09 (m, 1H), 1.52 (t, J=6.8 Hz, 3H), 1.23-1.27 (m, 2H), 1.01-1.06 (m, 2H).

The compounds given below were prepared by procedure similar to the one described above for compound ‘34b’ with appropriate variations of reactants, reaction conditions and quantities of reagents.

35b. N-(4-(4-chlorophenyl)thiazol-2-yl)-N-methylcyclopropanecarboxamide. MS: m/z 294 (M+1).

36b. N-(4-(4-chlorophenyl)thiazol-2-yl)-N-methylcyclopentanecarboxamide. MS: m/z 321 (M+1).

37b. N-(4-(4-chlorophenyl)thiazol-2-yl)-N-methylcyclohexanecarboxamide. MS: m/z 335 (M+1).

38b. N-(4-(4-chlorophenyl)thiazol-2-yl)-N-methyl propionamide. MS: m/z 281 (M+1).

39b. N-(4-(4-chlorophenyl)thiazol-2-yl)-2-methoxy-N-methylacetamide. MS: m/z 297 (M+1).

### Step 3: N-(4-(4-chlorophenyl)-5-(4-sulfamoylphenyl)thiazol-2-yl)-N-ethylcyclopropanecarboxamide (Compound 34)

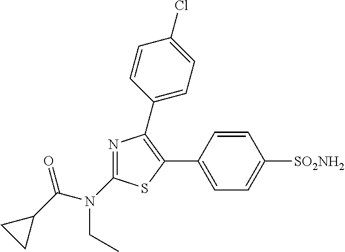 [SMILES: CCN(C(=O)C1CC1)c1nc(-c2ccc(Cl)cc2)c(-c2ccc(S(N)(=O)=O)cc2)s1]

4-bromobenzenesulfonamide (0.16 g, 0.68 mmol) and potassium acetate (0.122 g, 1.24 mmol) were added to a solution of N-(4-(4-chlorophenyl)thiazol-2-yl)-N-ethylcyclopropanecarboxamide (Compound 34b, 0.19 g, 0.62 mmol) in a DMA (5 ml) in a tube at 25° C. Nitrogen gas was bubbled through the reaction mixture for 15 minutes. Palladium acetate (0.014 g, 0.06 mmol) was then added to the reaction mixture under nitrogen atmosphere and the tube was sealed. The reaction mixture was heated at 150° C. for 4 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 40% ethyl acetate in hexanes as an eluent to obtain the title compound (0.04 g, 13.9%). MS: m/z 462 (M+1).

¹H NMR (DMSO-d₆, 400 MHz): δ 7.81 (dd, J=6.8, 1.6 Hz, 2H), 7.42-7.51 (m, 8H), 4.51 (q, J=6.8 Hz, 2H), 2.32-2.37 (m, 1H), 1.40 (t, J=6.8 Hz, 3H), 1.02-1.07 (m, 4H).

The following compounds were prepared according to the procedure described above for compound 34, with appropriate changes to the reactants.

N-(4-(4-chlorophenyl)-5-(4-sulfamoylphenyl)thiazol-2-yl)-N-methylcyclopropane carboxamide (Compound 35). MS: m/z 449 (M+1).

¹H NMR (DMSO-d₆, 400 MHz): δ 7.82 (d, J=8.4 Hz, 2H), 7.42-7.52 (m, 8H), 3.91 (s, 3H), 2.35-2.38 (m, 1H), 1.00-1.05 (m, 4H).

N-(4-(4-chlorophenyl)-5-(4-sulfamoylphenyl)thiazol-2-yl)-N-methylcyclopentane carboxamide (Compound 36). MS: m/z 477 (M+1).

¹H NMR (DMSO-d₆, 400 MHz): δ 7.82 (d, J=8.4 Hz, 2H), 7.41-7.52 (m, 8H), 3.76 (s, 3H), 3.42-3.46 (m, 1H), 1.97-1.99 (m, 2H), 1.80-1.82 (m, 2H), 1.67-1.76 (m, 4H).

N-(4-(4-chlorophenyl)-5-(4-sulfamoylphenyl)thiazol-2-yl)-N-methylcyclohexane carboxamide (Compound 37). MS: m/z 491 (M+1).

¹H NMR (DMSO-d₆, 400 MHz): δ 7.81 (d, J=8.4 Hz, 2H), 7.41-7.51 (m, 8H), 3.77 (s, 3H), 2.99-3.01 (m, 1H), 1.70-1.85 (m, 5H), 1.20-1.40 (m, 5H).

N-(4-(4-chlorophenyl)-5-(4-sulfamoylphenyl)thiazol-2-yl)-N-methylpropionamide (Compound 38). MS: m/z 436 (M+1).

¹H NMR (DMSO-d₆, 400 MHz): δ 7.81 (d, J=8.4 Hz, 2H), 7.41-7.51 (m, 8H), 3.71 (s, 3H), 2.79 (q, J=7.2 Hz, 2H), 1.12 (t, J=7.2 Hz, 3H).

N-(4-(4-chlorophenyl)-5-(4-sulfamoylphenyl)thiazol-2-yl)-2-methoxy-N-methylacetamide (Compound 39). MS: m/z 452 (M+1).

¹H NMR (DMSO-d₆, 400 MHz): δ 7.82 (d, J=8.4 Hz, 2H), 7.42-7.53 (m, 8H), 4.54 (s, 2H), 3.62 (s, 3H), 3.39 (s, 3H).

### Example 13

### N-(4-(4-chlorophenyl)-5-(4-sulfamoylphenyl)thiazol-2-yl)-N-cyclopropylacetamide (Compound 40)

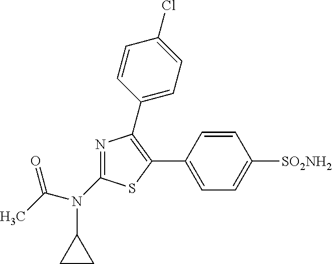 [SMILES: CC(=O)N(c1nc(-c2ccc(Cl)cc2)c(-c2ccc(S(N)(=O)=O)cc2)s1)C1CC1]

### Step 1: N-(4-bromothiazol-2-yl)-N-cyclopropylacetamide (40a)

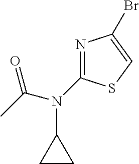

Sodium hydride (0.091 g, 60% dispersion in paraffin oil, 2.28 mmol) was added to a solution of 4-bromo-N-cyclopropylthiazol-2-amine (prepared according to the procedure reported in US 2005/153877, 0.50 g, 2.28 mmol) in THF (7 ml) at 0° C. To this reaction mixture acetyl chloride (0.215 g, 0.195 ml, 2.74 mmol) was added at 0° C. The reaction mixture was stirred at 25° C. for 0.5 hr. The progress of the reaction was monitored by TLC. The reaction mixture was quenched with water (10 ml). The mixture was then extracted with ethyl acetate (2×25 ml). The combined organic layer was dried over anhydrous Na₂SO₄. The solvent was evaporated under reduced pressure to obtain the title compound (0.30 g, 50.3%). MS: m/z 262 (M+1).

¹H NMR (CDCl₃, 400 MHz): δ 6.89 (s, 1H), 3.11-3.17 (m, 1H), 2.53 (s, 3H), 1.24-1.63 (m, 2H), 0.91-0.94 (m, 2H).

The compounds given below were prepared by procedure similar to the one described above for compound ‘40a’ with appropriate variations of reactants, reaction conditions and quantities of reagents.

41a. N-(4-bromothiazol-2-yl)-N-cyclopropylpropionamide. MS: m/z 275 (M+1).

42a. N-(4-bromothiazol-2-yl)-N-cyclopropylcyclopropane carboxamide. MS: m/z 287 (M+1).

### Step 2: N-(4-(4-chlorophenyl)thiazol-2-yl)-N-cyclopropylacetamide (40b)

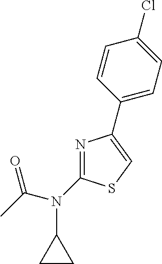 [SMILES: CC(=O)N(c1nc(-c2ccc(Cl)cc2)cs1)C1CC1]

(4-chlorophenyl) boronic acid (0.216 g, 1.38 mmol) and potassium carbonate (0.397 g, 2.87 mmol) were added to a solution of N-(4-bromothiazol-2-yl)-N-cyclopropylacetamide (Compound 40a, 0.3 g, 1.15 mmol) in a mixture of toluene:ethanol (3:7 ml) in a tube at 25° C. Nitrogen gas was bubbled through the reaction mixture for 15 minutes. Tetrakis(triphenylphosphine)(0)palladium(0.06 g, 0.057 mmol) was then added to the reaction mixture under nitrogen atmosphere and the tube was sealed. The reaction mixture was heated at 95-100° C. for 1 hr under stirring. The progress of the reaction was monitored by TLC. The reaction mixture was then cooled to 25° C. and filtered through celite. The celite cake was washed with ethanol (20 ml). The combined filtrate was concentrated under reduced pressure to obtain a crude product, which was then purified by column chromatography using 25% ethyl acetate in hexanes as an eluent to obtain the title compound (0.15 g, 44.6%). MS: m/z 293 (M+1).

¹H NMR (CDCl₃, 400 MHz): δ 7.86 (d, J=8.8 Hz, 2H), 7.38 (d, J=8.8 Hz, 2H), 7.18 (s, 1H), 3.18-3.23 (m, 1H), 2.55 (s, 3H), 1.31-1.36 (m, 2H), 0.97-1.01 (m, 2H).

The compounds given below were prepared by procedure similar to the one described above for compound ‘40b’ with appropriate variations of reactants, reaction conditions and quantities of reagents.

41b. N-(4-(4-chlorophenyl)thiazol-2-yl)-N-cyclopropylpropionamide. MS: m/z 307 (M+1).

42b. N-(4-(4-chlorophenyl)thiazol-2-yl)-N-cyclopropylcyclopropanecarboxamide. MS: m/z 319 (M+1).

### Step 3: N-(4-(4-chlorophenyl)-5-(4-sulfamoylphenyl)thiazol-2-yl)-N-cyclopropylacetamide (Compound 40)

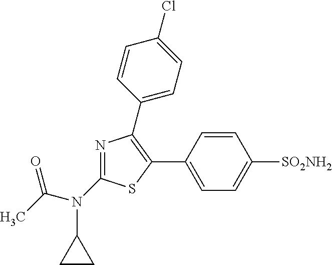 [SMILES: CC(=O)N(c1nc(-c2ccc(Cl)cc2)c(-c2ccc(S(N)(=O)=O)cc2)s1)C1CC1]

4-bromobenzenesulfonamide (0.174 g, 0.74 mmol) and potassium acetate (0.15 g, 1.53 mmol) were added to a solution of N-(4-(4-chlorophenyl)thiazol-2-yl)-N-cyclopropylacetamide (Compound 40b, 0.18 g, 0.61 mmol) in a DMA (4 ml) in a tube at 25° C. Nitrogen gas was bubbled through the reaction mixture for 15 minutes. Palladium acetate (0.014 g, 0.06 mmol) was then added to the reaction mixture under nitrogen atmosphere and the tube was sealed. The reaction mixture was heated at 140° C. for 8 hrs under stirring. The progress of the reaction was monitored by TLC. The reaction mixture was then cooled to 25° C. and filtered through celite. The celite cake was washed with ethyl acetate (20 ml). The combined filtrate was concentrated under reduced pressure to obtain a crude product, which was then purified by column chromatography using 55% ethyl acetate in hexanes as an eluent to obtain the title compound (0.026 g, 9.44%). MS: m/z 448 (M+1).

¹H NMR (CDCl₃, 400 MHz): δ 7.86 (d, J=8.0 Hz, 2H), 7.44-7.49 (m, 4H), 7.29-7.31 (d, J=8.8 Hz, 2H), 4.87 (bs-exchanges with D₂O, 2H), 3.18-3.19 (m, 1H), 2.58 (s, 3H), 1.35-1.36 (m, 2H), 1.03-1.04 (m, 2H).

The following compounds were prepared according to the procedure described above for compound 40, with appropriate changes to the reactants.

N-(4-(4-chlorophenyl)-5-(4-sulfamoylphenyl)thiazol-2-yl)-N-cyclopropyl propionamide (Compound 41). MS: m/z 462 (M+1),

¹H NMR (DMSO-d₆, 400 MHz): δ 7.81 (d, J=8.4 Hz, 2H), 7.41-7.51 (m, 8H), 3.17-3.19 (m, 1H), 2.92 (q, J=7.2 Hz, 2H), 1.19-1.24 (m, 2H), 1.12 (t, J=7.2 Hz, 3H), 0.99-1.03 (m, 2H).

N-(4-(4-chlorophenyl)-5-(4-sulfamoylphenyl)thiazol-2-yl)-N-cyclopropylcyclopropanecarboxamide (Compound 42). MS: m/z 475 (M+1).

¹H NMR (DMSO-d₆, 400 MHz): δ 7.80 (d, J=8.4 Hz, 2H), 7.43-7.50 (m, 8H), 2.67-2.74 (m, 2H), 1.29-1.30 (m, 2H), 1.01-1.08 (m, 6H).

### Example 14

### Pharmacological Screening

Compounds were tested in a cell-based real-time kinetic assay in human IMR-32 cells with native expression of α7 nAChR. The increase in intracellular Ca²⁺ levels was measured in a Fluorometric Imaging Plate Reader (FLIPR). Test compound and agonist solutions were made in assay buffer (HBSS, pH 7.4, 20 mM HEPES, and 10 mM CaCl₂). Briefly, cells were plated into Poly-D-Lysine coated back-walled clear-bottom 96-well microplates at a density of 80,000 to 100,000 cells/well and incubated at 37° C./5% CO₂ for 40-48 h prior to the experiment. For evaluation of compound mediated potentiation of agonist response, growth media was removed from the wells and 200 μl of FLIPR calcium 4 dye (Molecular Devices), reconstituted in assay buffer, and was added to the wells. After dye loading, microplates were incubated for 30 min at 37° C. and 30 min at room temperature and then directly transferred to the FLIPR. Baseline fluorescence was monitored for the first 10 to 30 s followed by the addition of 25 μl of test compound solution and subsequent monitoring of fluorescence changes for up to 10 min. This was followed by addition of 25 μl of agonist solution (PNU-282987, 10 μM) and measurement of fluorescence for 4 min. (Ramin Faghih et al. Journal of Medicinal Chemistry, 2009, 52, 3377-3384).

The compound induced fold increase in agonist response (fold PAM activity) was computed by dividing the maximum effect (Max-Min fluorescence) obtained with test compound in presence of agonist with the agonist-alone effect. EC₅₀ of the compound was calculated using GraphPad Prism software version 5.0, by plotting compound concentrations against fold PAM activity.

Fold activity at 1 μM concentration: compounds with activity between 1 to 5 folds are grouped as A, the compounds with activity between 5.1 folds and 15 folds are grouped as B and the compounds with activity above 15 folds are grouped as C.

Following table 1 provides fold activity of the compounds of the present invention

TABLE 1

| | Fold activation at | |
|---|---|---|
| Sr. No. | 1 μM conc. (Group) | Compound No. |
| 1 | A | 1, 2, 4, 5, 6, 8, 11, 13, 14, 15, 16, 17, |
| | | 21, 28, 29, 31, 32, 33, 37, 41, 42. |
| 2 | B | 3, 12, 18, 26, 30, 34, 36, 39. |
| 3 | C | 7, 9, 10, 19, 20, 22, 23, 24, 25, 27, 35, |
| | | 38, 40. |

1. A compound of formula (I), its tautomeric forms, its stereoisomers and its pharmaceutically acceptable salts, 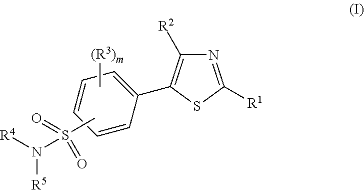
wherein,
R¹ is selected from —CR⁶(R⁷)—C(═O)—Y, —C(═O)—Z, —C(═O)R^{6a}R^{7a}, N(R^{6a})C(═O)R^{6b}, 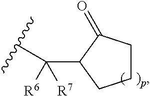
substituted- or unsubstituted-phenyl, and substituted- or unsubstituted-pyridyl; wherein substitutions on phenyl and pyridyl are selected from substituted- or unsubstituted-alkyl, halogen, and substituted- or unsubstituted-cycloalkyl;
Y is substituted- or unsubstituted-alkyl, substituted- or unsubstituted-cycloalkyl, and substituted- or unsubstituted-heterocyclyl; provided that when Y is selected as heterocyclyl the point of attachment of the said heterocyclyl is nitrogen;
Z is selected from substituted- or unsubstituted-pyridyl and substituted- or unsubstituted-cycloalkyl;
R² is selected as substituted- or unsubstituted-aryl;
R³ is selected independently at each occurrence from halogen, substituted- or unsubstituted-alkyl, perhaloalkyl, substituted- or unsubstituted-cycloalkyl, —OR^{8b}, and —C(═O)R^{8a};
R⁴ and R⁵ are independently selected from hydrogen, substituted- or unsubstituted-alkyl, and substituted- or unsubstituted-cycloalkyl;
R⁶ and R⁷ are selected from hydrogen, halogen, substituted- or unsubstituted-alkyl, and substituted- or unsubstituted-cycloalkyl; or
R⁶ and R⁷ groups and the carbon atoms to which they are attached together forming a carbocycle;
R^{6a} and R^{7a} are selected from hydrogen, substituted- or unsubstituted-alkyl, and substituted- or unsubstituted-cycloalkyl;
R^{6b} is selected from substituted- or unsubstituted-alkyl, perhaloalkyl, and substituted- or unsubstituted-cycloalkyl;
R^{8a} is selected from substituted- or unsubstituted-alkyl, perhaloalkyl, and substituted- or unsubstituted-cycloalkyl;
R^{8b} is selected from hydrogen, substituted- or unsubstituted-alkyl, perhaloalkyl, and substituted- or unsubstituted-cycloalkyl;
m is an integer selected from 0, 1, and 2;
n is an integer selected from 0, 1, and 2;
p is an integer selected from 1 and 2;
wherein,
when the alkyl group is a substituted alkyl group, the alkyl group is substituted with 1 to 4 substituents selected independently from oxo, halogen, nitro, cyano, perhaloalkyl, cycloalkyl, aryl, heteroaryl, heterocyclyl, —OR^{9b}, —SO₂R^{9a}, —C(═O)OR^{9a}, —OC(═O)R^{9a}, —C(═O)N(H)R⁹, —C(═O)N(alkyl)R⁹, —N(H)C(═O)R^{9a}, —N(H)R⁹, —N(alkyl)R⁹, —N(H)C(═O)N(H)R⁹, and —N(H)C(═O)N(alkyl)R⁹;
when the cycloalkyl and the carbocycle groups are substituted, each of them is substituted with 1 to 3 substituents selected independently from oxo, halogen, nitro, cyano, alkyl, perhaloalkyl, aryl, heteroaryl, heterocyclyl, —OR^{9b}, —SO₂R^{9c}, —C(═O)R^{9c}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d};
when the aryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heteroaryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, N(alkyl)alkyl, —N(H)alkyl, —NH₂, —SO₂-alkyl, —SO₂-perhaloalkyl, N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heterocyclyl group is substituted, it can be substituted either on a ring carbon atom(s) or on a ring hetero atom, and when it is substituted on a ring carbon atom(s), it is substituted with 1 to 3 substituents selected independently from halogen, nitro, cyano, oxo, alkyl, perhaloalkyl, cycloalkyl, aryl, heteroaryl, heterocyclyl, —OR^{9b}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d}; when the ‘heterocyclyl’ group is substituted on a ring nitrogen, it is substituted with a substituent selected from alkyl, cycloalkyl, aryl, heteroaryl, —SO₂R^{9c}, —C(═O)R^{9c}, —C(═O)N(H)R^{9d}, and —C(═O)N(alkyl)R^{9d};
R⁹ is selected from hydrogen, alkyl, cycloalkyl, aryl, heteroaryl, and heterocyclyl;
R^{9a} is selected from alkyl, perhaloalkyl, cycloalkyl, aryl, heteroaryl, and heterocyclyl;
R^{9b} is selected from hydrogen, alkyl, perhaloalkyl, cycloalkyl, aryl, heteroaryl, and heterocyclyl;
R^{9c} is selected from alkyl, perhaloalkyl, and cycloalkyl; and
R^{9d} is selected from hydrogen, alkyl, and cycloalkyl.

2. The compound of formula (I), its tautomeric forms, its stereoisomers, and its pharmaceutically acceptable salts, as claimed in claim 1, wherein R¹ is selected from substituted- or unsubstituted-phenyl, substituted- or unsubstituted-pyridyl, 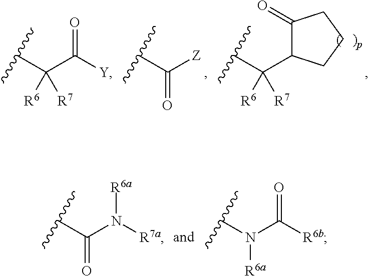
wherein, R⁶ and R⁷ are selected from hydrogen, alkyl and halogen; or R⁶ and R⁷ and the carbon to which they are attached together forming a cyclopropyl ring; Y is selected from cyclopropyl, pyrrolidinyl, 3-azabicyclo[3.1.0]hexanyl, bicyclo[3.1.0]hexanyl, and morpholinyl; Z is selected from cyclopentanyl and pyridyl; R^{6a} and R^{7a} are selected from hydrogen, substituted- or unsubstituted-alkyl, cyclopropyl, cyclopentyl, and cyclohexyl; R^{6b} is selected from substituted- or unsubstituted-alkyl, cyclopropyl, cyclopentyl, and cyclohexyl; and p is an integer selected from 1 or 2.

3. The compound of formula (I), its tautomeric forms, its stereoisomers, and its pharmaceutically acceptable salts, as claimed in claim 1, wherein R¹ is selected from

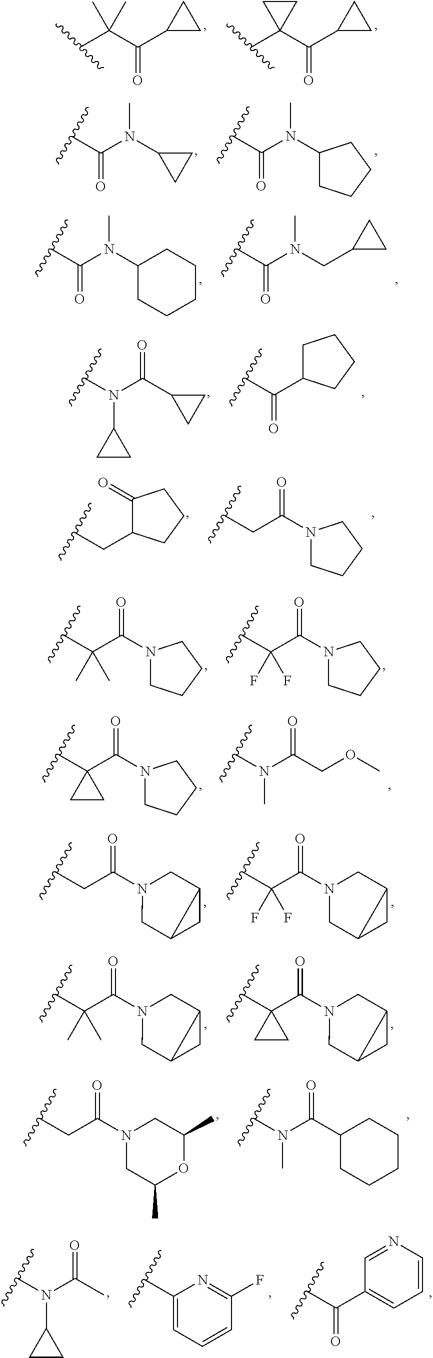

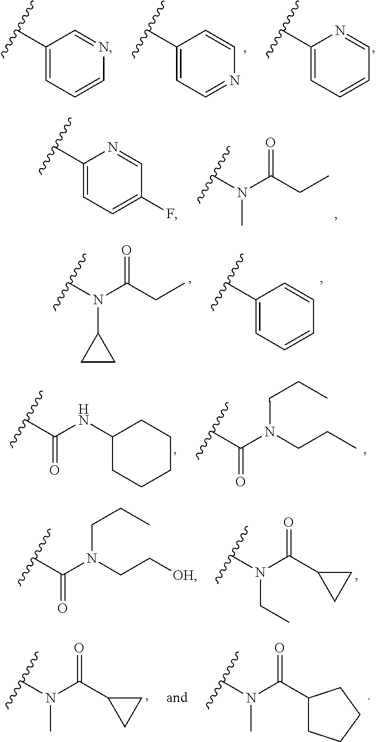

4. The compound of formula (I), its tautomeric forms, its stereoisomers, and its pharmaceutically acceptable salts, as claimed in claim 1, wherein R² is selected as aryl substituted with halogen.

5. The compound of formula (I), its tautomeric forms, its stereoisomers, and its pharmaceutically acceptable salts, as claimed in claim 1, wherein R² is selected as phenyl substituted with chloro.

6. The compound of formula (I), its tautomeric forms, its stereoisomers, and its pharmaceutically acceptable salts, as claimed in claim 1, wherein R³ is selected from halogens.

7. The compound of formula (I), its tautomeric forms, its stereoisomers, and its pharmaceutically acceptable salts, as claimed in claim 1, wherein R³ is selected from fluorine.

8. The compound of formula (I), its tautomeric forms, its stereoisomers, and its pharmaceutically acceptable salts, as claimed in claim 1, wherein m is selected from 0, 1, and 2.

9. The compound of formula (I), its tautomeric forms, its stereoisomers, and its pharmaceutically acceptable salts, as claimed in claim 1, wherein the compound is selected from:
4-(4-(4-chlorophenyl)-2-(cyclopentanecarbonyl)thiazol-5-yl)benzenesulfonamide;
4-(4-(4-chlorophenyl)-2-(cyclopentanecarbonyl)thiazol-5-yl)-3-fluorobenzenesulfonamide;
4-(4-(4-chlorophenyl)-2-picolinoylthiazol-5-yl)benzenesulfonamide;
4-(4-(4-chlorophenyl)-2-nicotinoylthiazol-5-yl)benzenesulfonamide;
4-(4-(4-chlorophenyl)-2-(1-cyclopropyl-2-methyl-1-oxopropan-2-yl)thiazol-5-yl)-3-fluorobenzenesulfonamide;
4-(4-(4-chlorophenyl)-2-(1-cyclopropyl-2-methyl-1-oxopropan-2-yl)thiazol-5-yl)benzenesulfonamide;
4-(4-(4-chlorophenyl)-2-(1-(cyclopropanecarbonyl)cyclopropyl)thiazol-5-yl)-3-fluorobenzenesulfonamide;
4-(4-(4-chlorophenyl)-2-((2-oxocyclopentyl)methyl)thiazol-5-yl)benzenesulfonamide;
4-(4-(4-chlorophenyl)-2-((2-oxocyclopentyl)methyl)thiazol-5-yl)-3-fluorobenzenesulfonamide;
4-(2-(2-(3-azabicyclo[3.1.0]hexan-3-yl)-2-oxoethyl)-4-(4-chlorophenyl)thiazol-5-yl)benzenesulfonamide;
(cis)-4-(4-(4-chlorophenyl)-2-(2-(2,6-dimethylmorpholino)-2-oxoethyl)thiazol-5-yl)benzenesulfonamide;
4-(4-(4-chlorophenyl)-2-(2-oxo-2-(pyrrolidin-1-yl)ethyl)thiazol-5-yl)benzenesulfonamide;
4-(2-(1-(3-azabicyclo[3.1.0]hexan-3-yl)-2-methyl-1-oxopropan-2-yl)-4-(4-chlorophenyl)thiazol-5-yl)benzenesulfonamide;
4-(4-(4-chlorophenyl)-2-(2-methyl-1-oxo-1-(pyrrolidin-1-yl)propan-2-yl)thiazol-5-yl)benzenesulfonamide;
4-(2-(1-(3-azabicyclo[3.1.0]hexane-3-carbonyl)cyclopropyl)-4-(4-chlorophenyl)thiazol-5-yl)benzenesulfonamide;
4-(4-(4-chlorophenyl)-2-(1-(pyrrolidine-1-carbonyl)cyclopropyl)thiazol-5-yl)benzenesulfonamide;
4-(2-(2-(3-azabicyclo[3.1.0]hexan-3-yl)-1,1-difluoro-2-oxoethyl)-4-(4-chlorophenyl)thiazol-5-yl)benzenesulfonamide;
4-(4-(4-chlorophenyl)-2-(1,1-difluoro-2-oxo-2-(pyrrolidin-1-yl)ethyl)thiazol-5-yl)benzenesulfonamide;
4-(4-(4-chlorophenyl)-2-(6-fluoropyridin-2-yl)thiazol-5-yl)benzenesulfonamide;
4-(4-(4-chlorophenyl)-2-(pyridin-3-yl)thiazol-5-yl)benzenesulfonamide;
4-(4-(4-chlorophenyl)-2-(pyridin-4-yl)thiazol-5-yl)benzenesulfonamide;
4-(4-(4-chlorophenyl)-2-(pyridin-2-yl)thiazol-5-yl)benzenesulfonamide;
4-(4-(4-chlorophenyl)-2-(pyridin-2-yl)thiazol-5-yl)-3-fluorobenzenesulfonamide;
4-(4-(4-chlorophenyl)-2-phenylthiazol-5-yl)benzenesulfonamide;
4-(4-(4-chlorophenyl)-2-(5-fluoropyridin-2-yl)thiazol-5-yl)benzenesulfonamide;
4-(4-chlorophenyl)-N-cyclopropyl-N-methyl-5-(4-sulfamoylphenyl)thiazole-2 carboxamide;
4-(4-chlorophenyl)-N-cyclopropyl-5-(2-fluoro-4-sulfamoylphenyl)-N-methylthiazole-2-carboxamide;
4-(4-chlorophenyl)-N-cyclopentyl-N-methyl-5-(4-sulfamoylphenyl)thiazole-2 carboxamide;
4-(4-chlorophenyl)-N-cyclohexyl-N-methyl-5-(4-sulfamoylphenyl)thiazole-2-carboxamide;
44-(4-chlorophenyl)-N-(cyclopropylmethyl)-N-methyl-5-(4-sulfamoylphenyl) thiazole-2-carboxamide;
4-(4-chlorophenyl)-N-cyclohexyl-5-(4-sulfamoylphenyl)thiazole-2-carboxamide;
4-(4-chlorophenyl)-N,N-dipropyl-5-(4-sulfamoylphenyl)thiazole-2-carboxamide;
4-(4-chlorophenyl)-N-(2-hydroxyethyl)-N-propyl-5-(4-sulfamoylphenyl)thiazole-2-carboxamide;
N-(4-(4-chlorophenyl)-5-(4-sulfamoylphenyl)thiazol-2-yl)-N-ethylcyclopropanecarboxamide;
N-(4-(4-chlorophenyl)-5-(4-sulfamoylphenyl)thiazol-2-yl)-N-methylcyclopropanecarboxamide;
N-(4-(4-chlorophenyl)-5-(4-sulfamoylphenyl)thiazol-2-yl)-N-methylcyclopentanecarboxamide;
N-(4-(4-chlorophenyl)-5-(4-sulfamoylphenyl)thiazol-2-yl)-N-methylcyclohexanecarboxamide;
N-(4-(4-chlorophenyl)-5-(4-sulfamoylphenyl)thiazol-2-yl)-N-methylpropionamide;
N-(4-(4-chlorophenyl)-5-(4-sulfamoylphenyl)thiazol-2-yl)-2-methoxy-N-methylacetamide;
N-(4-(4-chlorophenyl)-5-(4-sulfamoylphenyl)thiazol-2-yl)-N-cyclopropylacetamide;
N-(4-(4-chlorophenyl)-5-(4-sulfamoylphenyl)thiazol-2-yl)-N-cyclopropylpropionamide; and
N-(4-(4-chlorophenyl)-5-(4-sulfamoylphenyl)thiazol-2-yl)-N-cyclopropylcyclopropanecarboxamide.

10. A pharmaceutical composition comprising a compound of claim 1 a tautomeric form thereof, a stereoisomer thereof, or a pharmaceutically acceptable salt thereof, and a pharmaceutically acceptable carrier.

11. A method of preventing or treating a disease or its symptoms or a disorder mediated partially or completely by nicotinic acetylcholine receptors, said method comprising administering to a subject having or susceptible to said disease or its symptoms or disorder with a therapeutically effective amount of a compound of claim 1, a tautomeric form thereof, a stereoisomer thereof, or a pharmaceutically acceptable salt thereof.

12. A method of treating a disease or disorder or condition mediated partially or completely by nicotinic acetylcholine receptors in a subject in need thereof, comprising administering to the subject a therapeutically effective amount of a compound of formula (I), its tautomeric forms, its stereoisomers, or its pharmaceutically acceptable salts, 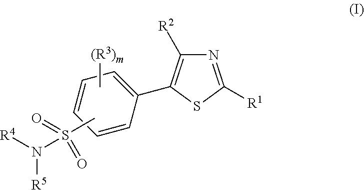
wherein,
R¹ is selected from —C(R)⁶(R⁷)—C(═O)—Y, —C(═O)—Z, —C(═O)NR^{6a}R^{7a}, —N(R^{6a})C(═O)R^{6b}, 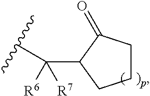
substituted- or unsubstituted-phenyl, and substituted- or unsubstituted-pyridyl; wherein substitutions on phenyl and pyridyl are selected from substituted- or unsubstituted-alkyl, halogen, and substituted- or unsubstituted-cycloalkyl;
Y is substituted- or unsubstituted-alkyl, substituted- or unsubstituted-cycloalkyl, and substituted- or unsubstituted-heterocyclyl; provided that when Y is selected as heterocyclyl the point of attachment of the said heterocyclyl is nitrogen;
Z is selected from substituted- or unsubstituted-pyridyl and substituted- or unsubstituted-cycloalkyl;
R² is selected as substituted- or unsubstituted-aryl;
R³ is selected independently at each occurrence from halogen, substituted- or unsubstituted-alkyl, perhaloalkyl, substituted- or unsubstituted-cycloalkyl, —OR^{8b}, and —C(═O)R^{8a};
R⁴ and R⁵ are independently selected from hydrogen, substituted- or unsubstituted-alkyl, and substituted- or unsubstituted-cycloalkyl;
R⁶ and R⁷ are selected from hydrogen, halogen, substituted- or unsubstituted-alkyl, and substituted- or unsubstituted-cycloalkyl; or
R⁶ and R⁷ groups and the carbon atoms to which they are attached together forming a carbocycle;
R^{6a} and R^{7a} are selected from hydrogen, substituted- or unsubstituted-alkyl, and substituted- or unsubstituted-cycloalkyl;
R^{6b} is selected from substituted- or unsubstituted-alkyl, perhaloalkyl, and substituted- or unsubstituted-cycloalkyl;
R^{8a} is selected from substituted- or unsubstituted-alkyl, perhaloalkyl, and substituted- or unsubstituted-cycloalkyl;
R^{8b} is selected from hydrogen, substituted- or unsubstituted-alkyl, perhaloalkyl, and substituted- or unsubstituted-cycloalkyl;
m is an integer selected from 0, 1, and 2;
n is an integer selected from 0, 1, and 2;
p is an integer selected from 1 and 2;
wherein,
when the alkyl group is a substituted alkyl group, the alkyl group is substituted with 1 to 4 substituents selected independently from oxo, halogen, nitro, cyano, perhaloalkyl, cycloalkyl, aryl, heteroaryl, heterocyclyl, —OR^{9b}, —SO₂R^{9a}, —C(═O)OR^{9a}, —OC(═O)R^{9a}, —C(═O)N(H)R⁹, —C(═O)N(alkyl)R⁹, —N(H)C(═O)R^{9a}, —N(H)R⁹, —N(alkyl)R⁹, —N(H)C(═O)N(H)R⁹, and —N(H)C(═O)N(alkyl)R⁹;
when the cycloalkyl and the carbocycle groups are substituted, each of them is substituted with 1 to 3 substituents selected independently from oxo, halogen, nitro, cyano, alkyl, perhaloalkyl, aryl, heteroaryl, heterocyclyl, —OR^{9b}, —SO₂R^{9c}, —C(═O)R^{9c}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d};
when the aryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, —N(alkyl)alkyl, —N(H)alkyl, —NH₂, —SO₂-alkyl, —SO₂-perhaloalkyl, N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heteroaryl group is substituted, it is substituted with 1 to 3 substituents selected independently from halogen, nitro, cyano, hydroxy, alkyl, perhaloalkyl, cycloalkyl, heterocyclyl, —O-alkyl, —O-perhaloalkyl, N(alkyl)alkyl, —N(H)alkyl, —NH₂, —SO₂-alkyl, —SO₂-perhaloalkyl, N(alkyl)C(═O)alkyl, —N(H)C(═O)alkyl, —C(═O)N(alkyl)alkyl, —C(═O)N(H)alkyl, —C(═O)NH₂, —SO₂N(alkyl)alkyl, —SO₂N(H)alkyl, and —SO₂NH₂;
when the heterocyclyl group is substituted, it can be substituted either on a ring carbon atom(s) or on a ring hetero atom, and when it is substituted on a ring carbon atom(s), it is substituted with 1 to 3 substituents selected independently from halogen, nitro, cyano, oxo, alkyl, perhaloalkyl, cycloalkyl, aryl, heteroaryl, heterocyclyl, —OR^{9b}, —C(═O)OR^{9c}, —OC(═O)R^{9c}, —C(═O)N(H)R^{9d}, —C(═O)N(alkyl)R^{9d}, —N(H)C(═O)R^{9c}, —N(H)R^{9d}, —N(alkyl)R^{9d}, —N(H)C(═O)N(H)R^{9d}, and —N(H)C(═O)N(alkyl)R^{9d}; when the ‘heterocyclyl’ group is substituted on a ring nitrogen, it is substituted with a substituent selected from alkyl, cycloalkyl, aryl, heteroaryl, —SO₂R^{9c}, —C(═O)R^{9c}, —C(═O)N(H)R^{9d}, and —C(═O)N(alkyl)R^{9d};
R⁹ is selected from hydrogen, alkyl, cycloalkyl, aryl, heteroaryl, and heterocyclyl;
R^{9a} is selected from alkyl, perhaloalkyl, cycloalkyl, aryl, heteroaryl, and heterocyclyl;
R^{9b} is selected from hydrogen, alkyl, perhaloalkyl, cycloalkyl, aryl, heteroaryl, and heterocyclyl;
R^{9c} is selected from alkyl, perhaloalkyl, and cycloalkyl; and
R^{9d} is selected from hydrogen, alkyl, and cycloalkyl.

13. The method of claim 11, wherein the disorder or condition or disease is selected from Alzheimer's disease, mild cognitive impairment, senile dementia, vascular dementia, dementia of Parkinson's disease, attention deficit disorder, attention deficit hyperactivity disorder, dementia associated with Lewy bodies, AIDS dementia complex, Pick's disease, dementia associated with Down's syndrome, Huntington's disease, cognitive deficits associated with traumatic brain injury, cognitive decline associated with stroke, poststroke neuroprotection, cognitive and sensorimotor gating deficits associated with schizophrenia, cognitive deficits associated with bipolar disorder, cognitive impairments associated with depression, acute pain, post-surgical or post-operative pain, chronic pain, inflammation, inflammatory pain, neuropathic pain, smoking cessation, need for new blood vessel growth associated with wound healing, need for new blood vessel growth associated with vascularization of skin grafts, and lack of circulation, arthritis, rheumatoid arthritis, psoriasis, Crohn's disease, ulcerative colitis, pouchitis, inflammatory bowel disease, celiac disease, periodontitis, sarcoidosis, pancreatitis, organ transplant rejection, acute immune disease associated with organ transplantation, chronic immune disease associated with organ transplantation, septic shock, toxic shock syndrome, sepsis syndrome, depression, and rheumatoid spondylitis.

14. The method of claim 11, wherein the disease or disorder or condition is selected from the group classified or diagnosed as major or minor neurocognitive disorders, or disorders arising due to neurodegeneration.

15. The method of claim 11, comprising administering a compound of formula (I), a tautomeric form thereof, a stereoisomer thereof, or a pharmaceutically acceptable salt thereof, in combination with or as adjunct to medications utilized in the treatment of attention deficit hyperactivity disorders, schizophrenia, cognitive disorders such as Alzheimer's disease, Parkinson's dementia, vascular dementia or dementia associated with Lewy bodies, or traumatic brain injury.

16. The method of claim 11, further comprising administering a compound of formula (I), a tautomeric form thereof, a stereoisomer thereof, or a pharmaceutically acceptable salt thereof, in combination with or as an adjunct to acetylcholinesterase inhibitors, disease modifying drugs or biologics for neurodegenerative disorders, dopaminergic drugs, antidepressants, or a typical or an atypical antipsychotic.

17-21. (canceled)
